(19) United States
(12) Patent Application Publication
(10) Pub. No.: US 2012/0010135 A1
(43) Pub. Date: Jan. 12, 2012

(54) SPIRO DERIVATIVES FOR THE MODULATION OF STEAROYL-COA DESATURASE

(75) Inventors: Natalie Dales, Arlington, MA (US); Julia Fonarev, Richmond (CA); Jianmin Fu, Coquitlam (CA); Zaihui Zhang, Vancouver (CA)

(73) Assignees: XENON PHARMACEUTICALS INC., Burnaby, BC (CA); NOVARTIS AG, Basel (CH)

(21) Appl. No.: 13/258,121

(22) PCT Filed: Mar. 30, 2010
(86) PCT No.: PCT/EP10/54234

§ 371 Date: Sep. 21, 2011

Related U.S. Application Data
(60) Provisional application No. 61/165,553, filed on Apr. 1, 2009.

(51) Int. Cl. A61K 31/438; A61P 3/00; A61P 3/10; A61P 5/48; A61P 3/08; A61P 3/04; A61P 3/06; A61P 9/12; A61P 7/00; A61P 19/06; A61P 7/02; A61P 17/00; A61P 17/10; A61P 17/06; A61P 17/08; A61P 17/02; A61K 38/28; A61K 31/60; C07D 491/107

(52) U.S. Cl. 514/6.5; 546/17; 514/278; 514/161

(57) ABSTRACT

The present invention provides spiro derivatives that modulate the activity of stearoyl-CoA desaturase. Methods of using such derivatives to modulate the activity of stearoyl-CoA desaturase and pharmaceutical compositions comprising such derivatives are also encompassed.

### FIELD OF THE INVENTION

The present invention relates generally to the field of inhibitors of stearoyl-CoA desaturase, such as spiro derivatives, and uses for such compounds in treating and/or preventing various human diseases, including those mediated by stearoyl-CoA desaturase (SCD) enzymes, preferably SCD1, especially diseases related to elevated lipid levels, cardiovascular disease, diabetes, obesity, metabolic syndrome, dermatological disorders and the like.

### BACKGROUND OF THE INVENTION

Acyl desaturase enzymes catalyze the formation of a double bond in fatty acids derived from either dietary sources or de novo synthesis in the liver. In mammals, at least three fatty acid desaturases exists, each with differing specificity: delta-9, delta-6, and delta-5, which introduce a double bond at the 9-10, 6-7, and 5-6 positions respectively.

Stearoyl-CoA desaturases (SCDs) act with cofactors (other agents) such as NADPH, cytochrome b5, cytochrome b5 reductase, Fe, and molecular O₂ to introduce a double bond into the C9-C10 position (delta 9) of saturated fatty acids, when conjugated to Coenzyme A (CoA). The preferred substrates are palmitoyl-CoA (16:0) and stearoyl-CoA (18:0), which are converted to palmitoleoyl-CoA (16:1) and oleyl-CoA (18:1), respectively. The resulting mono-unsaturated fatty acids are substrates for further metabolism by fatty acid elongases or incorporation into phospholipids, triglycerides, and cholesterol esters. A number of mammalian SCD genes have been cloned. For example, two genes have been identified in humans (hSCD1 and hSCD5) and four SCO genes have been isolated from mouse (SCD1, SCD2, SCD3, and SCD4). While the basic biochemical role of SCD has been known in rats and mice since the 1970s (Jeffcoat, R. et al., Eur J. Biochem. (1979), Vol. 101, No. 2, pp, 439-445; de Antueno, R. et al Lipids (1993), Vol. 28, No. 4, pp. 285-290), it has only recently been directly implicated in human disease processes.

The two human SCD genes have been previously described: hSCD1 by Brownlie et. al., PCT published patent application, WO 01/62954, and hSCD5 by Brownlie, PCT published patent application, WO 02/26944.

### SUMMARY OF THE INVENTION

The present invention presents new drug-like classes of compounds that are useful in modulating (e.g., inhibiting) SCD activity and regulating lipid levels, especially plasma lipid levels, and which are useful in the treatment of SCD-mediated diseases such as diseases related to dyslipidemia and disorders of lipid metabolism, especially diseases related to elevated lipid levels, cardiovascular disease, diabetes, obesity, metabolic syndrome, dermatological disorders and the like.

The present invention provides spiro derivatives that modulate (e.g., inhibit) the activity of stearoyl-CoA desaturase. Methods of using such derivatives to modulate the activity of stearoyl-CoA desaturase and pharmaceutical compositions comprising such derivatives are also encompassed.

Accordingly, in one aspect, the invention provides compounds of Formula (I)

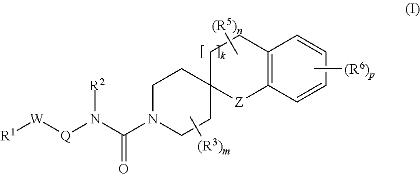

wherein Q is

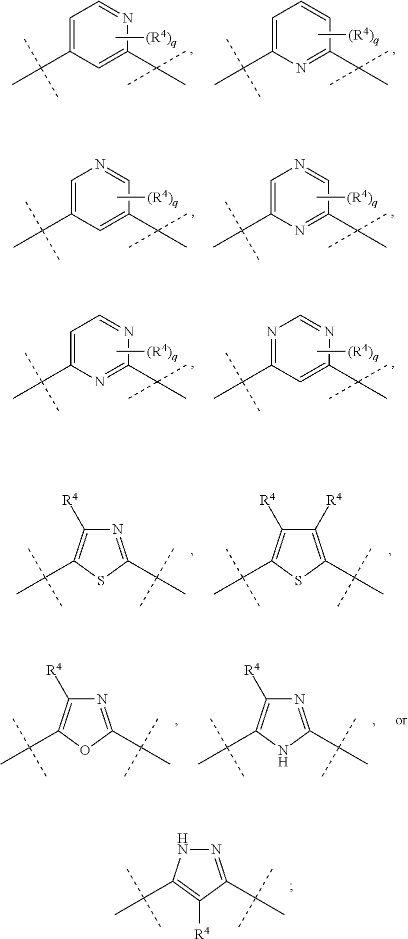

W is —N(R⁷)C(O)—, —C(O)N(R⁷)—, —N(R⁷)C(O)N(R⁷)—, —N(R⁷)S(O)ₜ—, —S(O)ₜN(R⁷)—, or a direct bond;
Z is —C(R⁴)ᵤ—, —C(O)—, —N(R⁷)—, —S(O)ₜ—, —O— or —S—;
k is 0 or 1;
m is 0 to 8;
n is 0, 1, 2, 3 or 4;
p is 0, 1, 2, 3 or 4;
q is 1, 2, or 3;
t is 1 or 2;
u is 1 or 2;
R¹ is hydrogen, alkyl, alkenyl, alkynyl, alkoxy, hydroxyalkyl, alkoxyalkyl, cycloalkyl, cycloalkylalkyl, aryl, haloalkyl, aralkyl, heterocyclyl, heterocyclylalkyl, heteroaryl, or heteroarylalkyl;
or R¹ is a multi-ring structure having 2 to 4 rings wherein the rings are independently cycloalkyl, heterocyclyl, aryl or heteroaryl and where some or all of the rings may be fused to each other;
R² is hydrogen, or alkyl;
R³ is independently alkyl, halo, haloalkyl, hydroxy, or
R⁴ is independently alkyl alkenyl, alkynyl, alkoxy, hydroxyalkyl, alkoxyalkyl, cycloalkyl, cycloalkylalkyl, heterocyclyl, aryl, aralkyl, heteroaryl, halo, haloalkyl, haloalkoxy, cyano, hydroxy or —N(R⁷)₂;
R⁵ is independently alkyl, halo, haloalkyl, hydroxy, cycloalkyl or —N(R⁷)₂;
or two R⁵ s on the same carbon atom form an oxo (═O)
R⁶ is independently alkyl, alkenyl, alkynyl, alkoxy, hydroxyalkyl, alkoxyalkyl, cycloalkyl, cycloalkylalkyl, heterocyclyl, aryl, aralkyl, heteroaryl, halo, haloalkyl, haloalkoxy, cyano, hydroxy or —N(R⁷)₂; and
R⁷ is independently hydrogen, alkyl, hydroxyalkyl, cycloalkylalkyl, aryl, heteroaryl, heterocyclyl or aralkyl; or
a pharmaceutically acceptable salt thereof or a prodrug thereof.

In another aspect, the invention provides methods of treating an SCD-mediated disease or condition in a mammal, preferably a human, wherein the methods comprise administering to the mammal in need thereof a therapeutically effective amount of a compound of the invention as set forth above.

In another aspect, the invention provides compounds or pharmaceutical compositions useful in treating, preventing and/or diagnosing a disease or condition relating to SCD biological activity such as the diseases encompassed by cardiovascular disorders and/or metabolic; syndrome (including dyslipidemia, insulin resistance and obesity).

In another aspect, the invention provides methods of preventing or treating a disease or condition related to elevated lipid levels, such as plasma lipid levels, especially elevated triglyceride or cholesterol levels, in a patient afflicted with such elevated levels, comprising administering to said patient a therapeutically or prophylactically effective amount of a composition as disclosed herein. The present invention also relates to novel compounds having therapeutic ability to reduce lipid levels in an animal, especially triglyceride and cholesterol levels.

In another aspect, the invention provides pharmaceutical compositions comprising the compounds of the invention as set forth above, and pharmaceutically acceptable excipients. In one embodiment, the present invention relates to a pharmaceutical composition comprising a compound of the invention in a pharmaceutically acceptable carrier and in an amount effective to modulate triglyceride level, or to treat diseases related to dyslipidemia and disorders of lipid metabolism, when administered to an animal, preferably a mammal, most preferably a human patient. In an embodiment of such composition, the patient has an elevated lipid level, such as elevated plasma triglycerides or cholesterol, before administration of said compound and said compound is present in an amount effective to reduce said lipid level.

In another aspect, the invention provides methods for treating a patient for, or protecting a patient from developing, a disease or condition mediated by stearoyl-CoA desaturase (SCD), which methods comprise administering to a patient afflicted with such disease or condition, or at risk of developing such disease or condition, a therapeutically effective amount of a compound that inhibits activity of SCD in a patient when administered thereto.

In another aspect, the invention provides methods for treating a range of diseases involving lipid metabolism and/or lipid homeostasis utilizing compounds identified by the methods disclosed herein. In accordance therewith, there is disclosed herein a range of compounds having said activity, based on a screening assay for identifying, from a library of test compounds, a therapeutic agent which modulates the biological activity of said SCD and is useful in treating a human disorder or condition relating to serum levels of lipids, such as triglycerides, VLDL, HDL, LDL, and/or total cholesterol.

### DETAILED DESCRIPTION OF THE INVENTION

### Definitions

Certain chemical groups named herein are preceded by a shorthand notation indicating the total number of carbon atoms that are to be found in the indicated chemical group. For example, C₇-C₁₂alkyl describes an alkyl group, as defined below, having a total of 7 to 12 carbon atoms; C₄-C₁₂cycloalkyl C₁-C₄alkyl describes a cycloalkylalkyl group, as defined below, having a total of 4 to 12 carbon atoms in the cycloalkyl group and 1 to 4 carbon atoms in the alkylene linker; and C₆-C₁₀arC₁-C₄alkyl describes an aralkyl group, as defined below, having a total of 6 to 10 carbon atoms in the aryl group and 1 to 4 carbon atoms in the alkylene linker. The total number of carbons in the shorthand notation does not include carbons that may exist in substituents of the group described.

Accordingly, as used in the specification and appended claims, unless specified to the contrary, the following terms have the meaning indicated:
“Cyano” refers to the —CN radical;
“Hydroxy” refers to the —OH radical;
“Nitro” refers to the —NO₂ radical;
“Amino” refers to the —N(R¹⁴)₂;
“Mercapto” refers to the —SR¹⁴ radical;
“Acid” refers to the —COOH radical;
“Trifluoromethyl” refers to the —CF₃ radical;
“Trifluoromethoxyl” refers to the —OCF₃ radical;

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms or one to four carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, an alkyl group may be optionally substituted by one or more of the following groups: alkyl, alkenyl, halo, haloalkyl, cyano, aryl, cycloalkyl, heterocyclyl, heteroaryl, —OR¹⁴, —OC(O)—R¹⁴, —N(R⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —SR¹⁶, —S(O)ₜR¹⁶ (where t is 1 to 2), —O—Si(R¹⁶)₃ and —S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl cycloalkyl, cycloalkylalkyl, aryl, aralkyl (e.g. tolyl), heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, having from two to twelve carbon atoms, preferably two to eight carbon atoms or two to six carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one or more of the following groups: alkyl, alkenyl, halo, haloalkyl, aryl, aralkyl, cycloalkyl, cycloalkylalkyl heterocyclyl, heterocyclylalkyl, heteroaryl, heteroarylalkyl, —OR¹⁴, —OC(O)—R¹⁴N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —SR¹⁶, —S(O)ₜR¹⁶ (where t is 1 to 2), and —S(O)ₜN(R¹⁴)₂ (where t is 1 to 2), where each R¹⁴ is independently hydrogen, alkyl, haloalkyl, cycloalkyl, cycloalkylalkyl, aryl aralkyl, heterocyclyl, heterocyclylalkyl, heteroaryl, or heteroarylalkyl; and each R¹⁶ is alkyl, cycloalkyl, cycloalkylalkyl, aryl, aralkyl, heterocyclyl, heterocyclylalkyl, heteroaryl, or heteroarylalkyl.

“Alkylene” refers to a straight or branched divalent hydrocarbon chain consisting solely of carbon and hydrogen atoms, having from one to twelve carbon atoms, preferably two to six carbon atoms, and linking the rest of the molecule to a radical group, e.g., methylene, ethylene, propylene, n-butylene, and the like. The alkylene is attached to the rest of the molecule through a single bond and to the radical group through a single bond. The points of attachment of the alkeylene to the rest of the molecule and to the radical group can be through one carbon or any two carbons within the chain. Unless stated otherwise specifically in the specification, an alkylene group may be optionally substituted by one or more of the following groups: alkyl, alkenyl, halo, haloalkyl, aryl, aralkyl, cycloalkyl, cycloalkylalkyl, heterocyclyl, heterocyclylalkyl, heteroaryl, heteroarylalkyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —SR¹⁶, —S(O)ₜR¹⁶ (where t is 1 to 2), and —S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms, preferably two to eight carbon atoms or two to six carbon atoms and which is attached to the rest of the molecule by a single bond. Unless stated otherwise specifically in the specification, an alkynyl group may be optionally substituted by one or more of the following groups: alkyl, alkenyl, halo, haloalkyl, aryl, aralkyl, cycloalkyl, cycloalkylalkyl, heterocyclyl, heterocyclylalkyl, heteroaryl, heteroarylalkyl, —OR¹⁴, —OC(O)—R¹⁴N(R¹⁴)₂, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —SR¹⁶, —S(O)ₜR¹⁶ (where t is 1 to 2), and —S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalky, and each R¹⁶ is alkyl, cycloalkyl, cycloalkylalkyl, aryl, aralkyl, heterocyclyl, heterocyclylalkyl, heteroaryl or heteroarylalkyl.

“Alkenylene” and “alkenylene chain” refer to a straight or branched divalent hydrocarbon chain linking the rest of the molecule to a radical group, consisting solely of carbon and hydrogen, containing at least one double bond and having from two to twelve carbon atoms or two to six carbon atoms, e.g., ethenylene, propenylene, n-butenylene, and the like. Unless stated otherwise specifically in the specification, an alkenylene chain may be optionally substituted by one or more of the following groups: alkyl, alkenyl, halo, cyano, aryl, cycloalkyl, heterocyclyl, heteroaryl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁴) (where t is 1 to 2), —SR¹⁶, —S(O)ₜR¹⁶ (where t is 1 to 2), and —S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, cycloalkyl, cycloalkylalkyl, aryl, aralkyl, heterocyclyl, heterocyclylalkyl, heteroaryl or heteroarylalkyl.

“Alkynylene” and “Alkynylene chain” refer to a straight or branched divalent hydrocarbon chain linking the rest of the molecule to a radical group, consisting solely of carbon and hydrogen, containing at least one triple bond and having from two to twelve carbon atoms or two to six carbon atoms, e.g. propynylene, n-butynylene, and the like. Unless stated otherwise specifically in the specification, an alkynylene chain may be optionally substituted by one or more of the following groups: alkyl, alkenyl, halo, cyano, aryl, cycloalkyl, heterocyclyl, heteroaryl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —SR¹⁶, —S(O)ₜOR¹⁶ (where t is 1 to 2), —S(O)ₜR¹⁶ (where t is 1 to 2), and —S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, cycloalkyl, cycloalkylalkyl, aryl, aralkyl, heterocyclyl, heterocyclylalkyl, heteroaryl or heteroarylalkyl.

“Alkoxy” refers to a radical of the formula —ORₐ where Rₐ is an alkyl radical as generally defined above. The alkyl part of the alkoxy radical may be optionally substituted as defined above for an alkyl radical.

“Alkoxyalkyl” refers to a radical of the formula —R_{b}—O—Rₐ where R_{b} is an alkylene as defined above, and Rₐ is an alkyl radical as defined above. The oxygen atom may be bonded to any carbon in the alkyl or alkylene radical. Each alkyl part of the alkoxyalkyl radical may be optionally substituted as defined above for an alkyl group. Each alkylene part of the alkoxyalkyl radical may be optionally substituted as defined above for an alkylene group.

“Aryl” refers to aromatic monocyclic or multicyclic, preferably mono- or bi-cyclic, hydrocarbon ring system consisting only of hydrogen and carbon and containing from six to nineteen carbon atoms, preferably six to ten carbon atoms, where the ring system may be partially saturated provided that at least one ring in the ring system is aromatic. Aryl groups include but are not limited to groups such as fluorenyl, phenyl and naphthyl. Unless stated otherwise specifically in the specification, the term “aryl” or the prefix “ar-” (such as in “aralkyl”) is meant to include aryl radicals optionally substituted by one or more substituents selected from the group consisting of alkyl, alkenyl, alkynyl, halo, haloalkyl, cyano, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶) (where t is 1 to 2), —R¹⁵—SR¹⁶, —R¹⁵—S(O)ₜR¹⁶ (where t is 1 to 2), and —R¹⁵—S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl, or heteroarylalkyl.

“Aralkyl” refers to a radical of the formula —R_{b}R_{d} where R_{b}, is an alkylene chain as defined above and R_{d} is one or more aryl radicals as defined above, e.g., benzyl, diphenylmethyl, phenylethyl, phenylpropyl, and the like. The aryl part of the aralkyl radical may be optionally substituted as described above for an aryl group. The alkylene chain of the aralkyl radical may be optionally substituted as defined above for an alkylene chain.

“Aralkenyl” refers to a radical of the formula —RₑR_{d} where Rₑ is an alkenylene chain as defined above and R_{d} is one or more aryl radicals as defined above. The aryl part of the aralkenyl radical may be optionally substituted as described above for an aryl group. The alkenylene chain of the aralkenyl radical may be optionally substituted as defined above for an alkenylene chain.

“Aryloxy” refers to a radical of the formula —OR_{d} where R_{d} is an aryl group as defined above. The aryl part of the aryloxy radical may be optionally substituted as defined above.

“Cycloalkyl” refers to a stable non-aromatic monocyclic or bicyclic hydrocarbon radical consisting solely of carbon and hydrogen atoms, having from three to fifteen carbon atoms, preferably having from three to twelve carbon atoms or from three to seven carbon atoms, and which is saturated or unsaturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents selected from the group consisting of alkyl, alkenyl, alkynyl, halo, haloalkyl, cyano, aryl aralkyl, cycloalkyl, cycloalkylalkyl, heterocyclyl, heterocyclylalkyl, heteroaryl, heteroarylalkyl, —R¹⁵—OR, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶) (where t is 1 to 2), —R¹⁵—SR¹⁶, —R¹⁵—S(O)ₜR¹⁶ (where t is 1 to 2), and —R¹⁵—S(O)ₜN(R¹⁴)₂ (where t is 1 to 2), where each R₁₄ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl, or heteroarylalkyl.

“Cycloalkylalkyl” refers to a radical of the formula —R_{b}R_{f} where R_{b} is an alkylene chain as defined above and R_{f} is a cycloalkyl radical as defined above. The cycloalkyl part of the cycloalkyl radical may be optionally substituted as defined above for a cycloalkyl radical. The alkylene chain of the cycloalkyl radical may be optionally substituted as defined above for an alkylene chain.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkoxy” refers to an alkoxy radical, as defined above, that is substituted by one or more halo radicals, as defined above. The alkoxy part of the haloalkoxy radical may be optionally substituted as defined above for an alkoxy group.

“Heterocyclyl” refers to a stable 3- to 18-membered non-aromatic ring radical which consists of carbon atoms and from one to five heteroatoms selected from the group consisting of nitrogen, oxygen and sulfur, preferably having from two to ten carbon atoms. For purposes of this invention, the heterocyclyl radical may be a monocyclic, bicyclic or tricyclic ring system, which may include fused or bridged ring systems, which may be partially unsaturated; and the nitrogen, carbon or sulfur atoms in the heterocyclyl radical may be optionally oxidized; the nitrogen atom may be optionally alkylated/substituted; and the heterocyclyl radical may be partially or fully saturated. Examples of such heterocyclyl radicals include, but are not limited to, dioxolanyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl, homopiperidinyl, homopiperazinyl, and quinuclidinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, cyano, oxo, thioxo, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁴—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶—R¹⁵—N(R¹⁴)(S(O)ₜR¹⁶) (where t is 1 to 2), R¹⁵—SR¹⁶, —R¹⁵—S(O)R¹⁶ (where t is 1 to 2), and —R¹⁵(O)N(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl, or heteroarylalkyl, and where each of the above substituents is unsubstituted.

“Heterocyclylalkyl” refers to a radical of the formula —R_{b}R_{g} where R_{b} is an alkylene chain as defined above and R_{g} is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkylene chain. The heterocyclyl part of the heterocyclylalkyl radical may be optionally substituted as defined above for a heterocyclyl group.

“Heteroaryl” refers to a 5- to 18-membered aromatic ring radical which consists of carbon atoms and from one to five heteroatoms selected from the group consisting of nitrogen, oxygen and sulfur. For purposes of this invention, the heteroaryl radical may be a monocyclic, bicyclic or tricyclic ring system, which may include fused or bridged ring systems, which may be partially saturated provided that at least one ring in the ring system is aromatic; and the nitrogen, carbon or sulfur atoms in the heteroaryl radical may be optionally oxidized; the nitrogen atom may be optionally alkylated/substituted. Examples include but are not limited to acridinyl, benzimidazolyl, benzthiazolyl, benzindolyl, benzothiadiazolyl, benzonaphthofuranyl, benzoxazolyl, benzodioxolyl, benzodioxinyl, benzopyranyl, benzopyranonyl, benzofuranyl, benzofuranonyl, benzothienyl, benzo[b]thiophenyl, benzothiophenyl, benzotriazolyl, benzo[4,6]imidazo[1,2-a]pyridinyl, benzo[c][1,2,5]oxadiazolyl, benzo[c][1,2,5]thiadiazolyl, carbazolyl, cinnolinyl, dibenzofuranyl, furanyl, furanonyl, isoquinolinyl, isothiazolyl, imidazolyl, indolyl, indazolyl, isoindolyl indolinyl isoindolinyl, indolizinyl, isoxazolyl, naphthyridinyl, oxadiazolyl, oxazolyl phenazinyl, phenothiazinyl, phenoxazinyl, phthalazinyl, pterndinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, thiazolyl thiadiazolyl, triazolyl, tetrazolyl, triazinyl, and thiophenyl. Unless stated otherwise specifically in the specification, the term “heteroaryl” is meant to include heteroaryl radicals as defined above which are optionally substituted by one or more substituents selected from the group consisting of alkyl, alkenyl, alkynyl, halo, haloalkyl, cyano, oxo, thioxo, nitro, aryl, aralkyl, cycloalkyl, cycloalkylalkyl, heterocyclyl, heterocyclyl alkyl heteroaryl, heteroarylalkyl, —R¹⁵, —OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶) (where t is 1 to 2), R¹⁵—SR¹⁶, —R¹⁵—S(O)ₜR¹⁶ (where t is 1 to 2), and —R¹⁵—S(O)ₜN(R¹⁴)₂ (where t is 1 to 2), where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain, and each R¹⁶ is alkyl, haloalkyl, cycloalkyl, cycloalkylalkyl, aryl, aralkyl, heterocyclyl, heterocyclylalkyl, heteroaryl, or heteroarylalkyl.

“Heteroarylalkyl” refers to a radical of the formula —R_{b}Rₕ where Rₕ is an alkylene chain as defined above and Rₕ is a heteroaryl radical as defined above. The heteroaryl part of the heteroarylalkyl radical may be optionally substituted as defined above for a heteroaryl group. The alkylene chain of the heteroarylalkyl radical may be optionally substituted as defined above for an alkylene chain.

“Hydroxyalkyl” refers to a radical of the formula —R_{b}OH where R_{b} is an alkylene radical as defined above. The hydroxy group may be attached to the alkylene chain on any carbon within the alkylene chain. The alkylene chain of the hydroxyalkyl group may be optionally substituted as defined above for an alkylene chain.

“A multi-ring structure” refers to a multicyclic ring system comprised of two to four rings wherein the rings are independently selected from cycloalkyl, aryl, heterocyclyl or heteroaryl as defined above. Each cycloalkyl may be optionally substituted as defined above for a cycloalkyl group. Each aryl may be optionally substituted as defined above for an aryl group. Each heterocyclyl may be optionally substituted as defined above for a heterocyclyl group. Each heteroaryl may be optionally substituted as defined above for a heteroaryl group. The rings may be attached to each other through direct bonds or some or all of the rings may be fused to each other.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention.

Prodrugs are typically rapidly transformed in vivo to yield the parent compound of the invention, for example, by hydrolysis in blood or conversion in the gut or liver. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)).

A discussion of prodrugs is provided in Higuchi, T., et al “Pro-drugs as Novel Delivery Systems,” A.C.S. Symposium Series, Vol. 14, and in Bioreversible Carriers in Drug Design, ed. Edward B. Roche, Anglican Pharmaceutical Association arid Pergamon Press, 1987.

The term “prodrug” is also meant to include any covalently bonded carriers which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto or acid group is bonded to any group that when the prodrug of the compound of the invention is administered to a mammalian subject, cleaves to form a free hydroxy, free amino or free mercapto or acid group, respectively. Examples of prodrugs include, but are not limited to, acetate, formate and benzoate derivatives of alcohol or amides of amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent. A skilled artisan will recognize unstable combinations of substituents.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts,

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid.

2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphonic acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to the sodium, potassium, lithium, ammonium calcium, magnesium, iron, zinc, copper, manganese, aluminum salts and the like. Preferred inorganic salts are the ammonium, sodium potassium, calcium, and magnesium salts. Salts derived from organic bases include, but are not limited to salts of primary, secondary and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients thereof.

“Therapeutically effective amount” refers to that amount of a compound of the invention which, when administered to a mammal, preferably a human, is sufficient to effect treatment, as defined below, of an SCD-mediated disease or condition in the mammal, preferably a human. The amount of a compound of the invention which constitutes a “therapeutically effective amount” will vary depending on the compound, the condition and its seventy, and the age and body weight of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or disorder of interest, and includes: (i) preventing the disease or condition from occurring in a mammal, in particular, when such mammal is predisposed to the condition but has not yet been diagnosed as having it, (ii) inhibiting the disease or condition, i.e., arresting its development, (iii) relieving the disease or condition, i.e., causing regression of the disease or condition; or (iv) reducing the risk of developing the disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. Formulae (I), (II), (III), (IV), and (V) are meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, Formulae (I), (II), (III), (IV), and (V) are meant to include all tautomeric forms.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

The present invention includes all pharmaceutically acceptable isotopically-labeled compounds of the invention wherein one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³¹P and ³²P, and sulphur, such as ³⁵S. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Isotopically-labeled compounds of the invention can generally be prepared by conventional techniques known to those skilled in the art or by processes analogous to those described in the accompanying Examples and Preparations Sections using an appropriate isotopically-labeled reagent in place of the non-labeled reagent previously employed.

The chemical naming protocol and structure diagrams used herein employ and rely on the chemical naming features as utilized by Chemdraw versions 10.0 or 11.0 (available from Cambridgesoft Corp., Cambridge, Mass.) or ISIS draw version 2.5 (available from MDL information systems).

Throughout this specification and in the claims that follow, unless the context requires otherwise, the word “comprise”, or variations such as “comprises”, “comprised of”, “comprising” or “comprising of”, will be understood to imply the inclusion of a stated integer or step or group of integers or steps but not the exclusion of any other integer or step or group of integers or steps and therefore inclusive and open-ended in that additional elements.

### Embodiments of the Invention

In one embodiment the invention provides compounds of Formula (I)

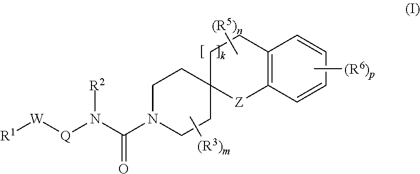

wherein Q is

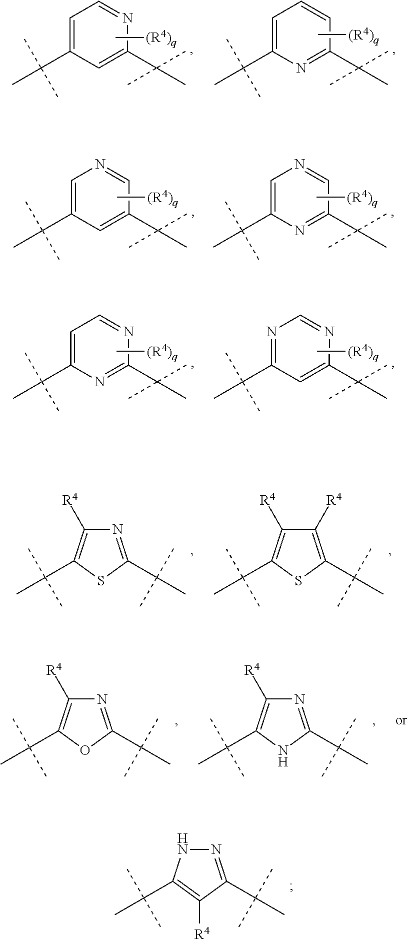

W is —N(R⁷)C(O)—, —C(O)N(R)—, —N(R⁷)C(O)N(R⁷)—, —N(R⁷)S(O)ₜ—, —S(O)ₜN(R⁷)—, or a direct bond;
Z is —C(R₄)ᵤ—, —C(O)—, —O—, —N(R⁷)—, —S(O)ₜ—, —O— or —S—;
k is 0 or 1;
m is 0 to 8;
n is 0, 1, 2, 3 or 4;
p is 0, 1, 2, 3 or 4;
q is 1, 2, or 3;
t is 1 or 2;
u is 1 or 2;
R¹ is hydrogen, alkyl, alkenyl, alkynyl, alkoxy, hydroxyalkyl, alkoxyalkyl, cycloalkyl, cycloalkylalkyl, aryl, haloalkyl, aralkyl, heterocyclyl, heterocyclylalkyl, heteroaryl, or heteroarylalkyl;
or R¹ is a multi-ring structure having 2 to 4 rings wherein the rings are independently cycloakyl, heterocyclyl, aryl or heteroaryl and where some or all of the rings may be fused to each other;
R² is hydrogen, or alkyl;
R³ is independently alkyl, halo, haloalkyl, hydroxy, or —N(R⁷)₂;
R⁴ is independently alkyl, alkenyl, alkynyl, alkoxy, hydroxyalkyl, alkoxyalkyl, cycloalkyl, cycloalkylalkyl, heterocyclyl, aryl, aralkyl, heteroaryl, halo, haloalkyl, haloalkoxy, cyano, hydroxy or —N(R⁷)₂;
R⁵ is independently alkyl, halo, haloalkyl, hydroxy, cycloalkyl, or —N(R⁷)₂; or two R⁵'s on the same carbon atom form an oxo (═O);
R⁶ is independently alkyl, alkenyl, alkynyl, alkoxy, hydroxyalkyl, alkoxyalkyl, cycloalkyl cycloalkylalkyl, heterocyclyl, aryl, aralkyl, heteroaryl, halo, haloalkyl, haloalkoxy, cyano, hydroxy or —N(R⁷)₂; and
R⁷ is independently hydrogen, alkyl, hydroxyalkyl, cycloalkyl, cycloalkylalkyl, aryl, heteroaryl, heterocyclyl or aralkyl; or
a pharmaceutically acceptable salt thereof or a prodrug thereof.

Various embodiments of the invention are described below. It will be appreciated that the features specified in each embodiment may be combined with other specified features, to provide further embodiments.

In another embodiment the invention provides compounds of Formula (I), wherein
R¹ is hydrogen, C₁-C₄alkyl, C₂-C₆-alkenyl, C₂-C₆alkynyl, C₁-C₆-alkoxy, hydroxylC₁-C₄alkyl, C₁-C₆alkoxyC₁-C₄alkyl, C₃-C₇cycloalkylC₃-C₇cycloalkylC₁-C₄alkyl, C₆-C₁₀aryl, haloC₁-C₄alkyl, C₆-C₁₀arC₁-C₄alkyl, C₁-C₁₀heterocyclyl, C₁-C₁₀heterocyclylC₁-C₄alkyl, C₁-C₁₀heteroaryl, or C₁-C₁₀heteroarylC₁-C₄alkyl;
or is a multi-ring structure having 2 to 4 rings wherein the rings are independently cycloalkyl, heterocyclyl, aryl or heteroaryl and where some or all of the rings may be fused to each other;
R² is hydrogen, or C₁-C₄alkyl;
R³ is independently C₁-C₄alkyl, halo, haloC₁-C₄alkyl, hydroxy, or —N(R⁷)₂;
R⁴ is independently C₁-C₄alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₁-C₁₀heterocyclyl, C₆-C₁₀aryl, C₆-C₁₀arC₁-C₆alkyl, C₁-C₁₀heteroaryl, halo, trifluoromethyl, trifluoromethoxy, cyano, hydroxy, or —N(R⁷)₂;

R⁵ is independently C₁-C₄alkyl, halo, haloC₁-C₄alkyl, hydroxy, or —N(R⁷)₂;
or two R⁵'s on the same carbon atom form an oxo (═O);
R⁶ is independently C₁-C₄alkyl, C₂-C₆alkenyl, C₂-C₆alkynyl, C₁-C₆alkoxy, hydroxyC₁-C₄alkyl, alkoxyC₁-C₄alkyl, C₃-C₇cycloalkyl, C₃-C₇cycloalkylC₁-C₄alkyl, C₁-C₁₀heterocyclyl, C₆-C₁₀aryl, C₆-C₁₀arC₁-C₄alkyl, C₁-C₁₀heteroaryl, halo, trifluoromethyl, trifluoromethoxy, cyano, hydroxy or —N(R⁷)₂; and
R⁷ is independently hydrogen, C₁-C₄alkyl, hydroxyC₁-C₄alkyl, C₃-C₇cycloalkyl, C₃-C₇cycloalkylC₁-C₄alkyl, C₆-C₁₀aryl, C₁-C₁₀heteroaryl, C₁-C₁₀heterocyclyl or C₆-C₁₀arC₁-C₄alkyl.

A subgroup of for the compounds of the invention is represented by Formula (I), wherein Q is

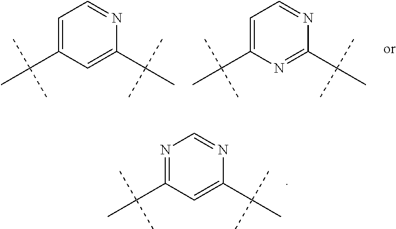

A subgroup of Q for the compounds of the invention is represented by Formula (I) wherein Q is

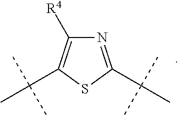

A subgroup of R¹ and W for the compounds of the invention is represented by Formula (I), wherein W is —N(R⁷)C(O)—, and R¹ is hydrogen, or C₁-C₄alkyl.

A subgroup of R¹ and W for the compounds of the invention is represented by Formula (I), wherein W is —N(R⁷)C(O)— and R¹ is C₁-C₅heteroarylC₁-C₄alkyl.

A subgroup of R¹ for the compounds of the invention is represented by Formula (I), wherein W is a direct bond or —N(R⁷)C(O)—, and R¹ is

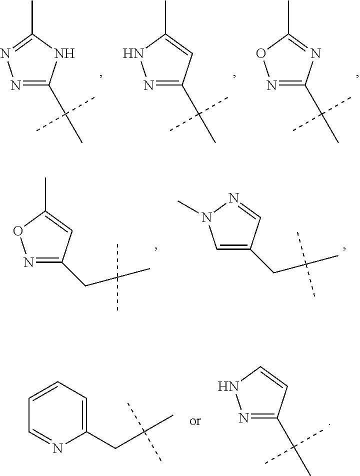

A subgroup of R² for the compounds of the invention is represented by Formula (I), wherein R² is hydrogen.

A subgroup of R² for the compounds of the invention is represented by Formula (I), wherein R² is C₁-C₄alkyl.

A subgroup of R³ for the compounds of the invention is represented by Formula (I) wherein R³ is methyl, ethyl, hydroxy, or fluoro.

A subgroup of R⁴ for the compounds of the invention is represented by Formula (I), wherein R⁴ is C₁-C₄alkyl.

A subgroup of R⁵ for the compounds of the invention is represented by Formula (I), wherein R⁵ is C₁-C₄alkyl, hydroxy, or two R⁵'s on the same carbon atom form an oxo (═O).

A subgroup of R⁵ for the compounds of the invention is represented by Formula (I), wherein two R⁵'s on the same carbon atom form an oxo (═O).

A subgroup of R⁵ for the compounds of the invention is represented by Formula (I), wherein R⁷ is C₁-C₄alkyl, C₃-C₇cycloalkyl, chloro, fluoro, trifluoromethyl, or cyano.

A subgroup of R⁷ for the compounds of the invention is represented by Formula wherein R⁷ is hydrogen, or C₁-C₄alkyl.

In another embodiment the invention provides compounds represented by Formula (II):

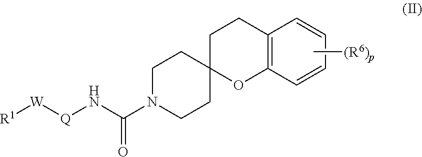

wherein Q is

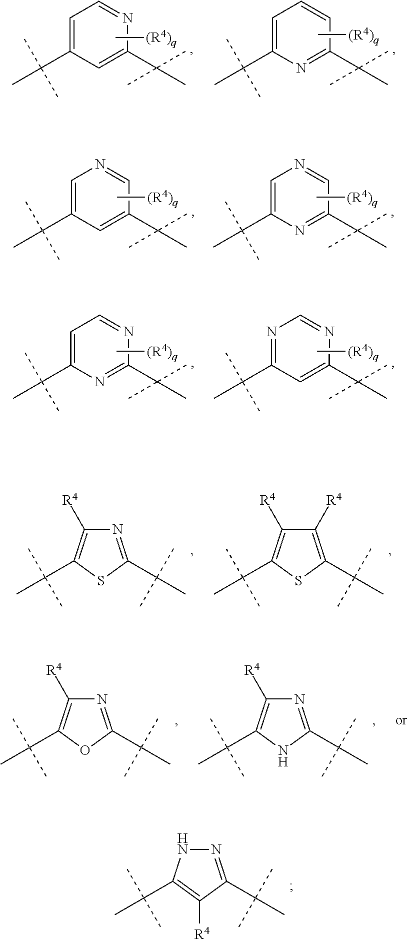

W is —N(R⁷)C(O)—, —C(O)N(R⁷)—, or a direct bond;
p is 0, 1, 2, 3, or 4;
q is 1, 2, or 3;
R¹ is hydrogen, alkyl, alkenyl, alkynyl, alkoxy, hydroxyalkyl, alkoxyalkyl, cycloalkyl; cycloalkylalkyl, aryl, haloalkyl, aralkyl, heterocyclyl, heterocyclylalkyl, heteroaryl, or heteroarylalkyl;
R⁴ is independently alkyl, alkoxy, hydroxyalkyl, alkoxyalkyl, cycloalkyl, halo, trifluromethyl, trifluoromethoxy, cyano, hydroxy, or
R⁶ is independently C₁-C₄alkyl, halo, trifluoromethyl, trifluoromethoxy, cyano, hydroxy, or —N(R⁷)₂; and
R⁷ is independently hydrogen, or C₁-C₄alkyl; or
a pharmaceutically acceptable salt thereof or a prodrug thereof.

A subgroup of R¹ for the compounds of the invention is represented by Formula (II), wherein R¹ is hydrogen, C₁-C₄alkyl, C₂-C₆alkenyl; C₂-C₆alkynyl, C₁-C₆alkoxy, hydroxylC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, C₆-C₁₀arC₁-C₄alkyl, C₁-C₁₀heterocyclyl, C₁-C₁₀heterocyclylC₁-C₄alkyl, C₁-C₁₀heteroaryl, or C₁-C₁₀heteroarylC₁-C₄alkyl.

A subgroup of Q for the compounds of the invention is represented by Formula (II), wherein Q is

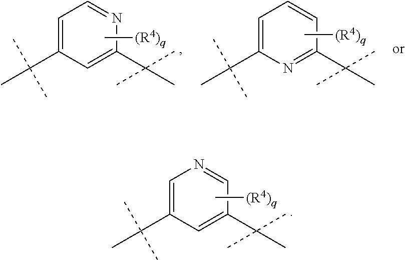

A subgroup of Q for the compounds of the invention is represented by Formula (II), wherein Q is

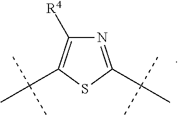

A subgroup of R⁴ for the compounds of the invention is represented by Formula (II), wherein R⁴ is C₁-C₄alkyl, C₁-C₆alkoxy, hydroxyC₁-C₄alkyl, C₁-C₆alkoxyC₁-C₄alkyl, C₃-C₇cycloalkyl, halo, trifluoromethyl, trifluoromethoxy; cyano, hydroxy; or —N(R⁷)₂.

In another embodiment the invention provides compounds represented by Formula (III):

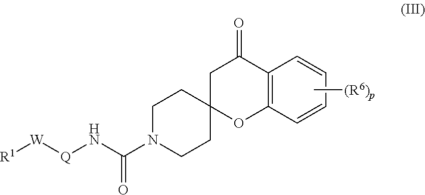

wherein Q is

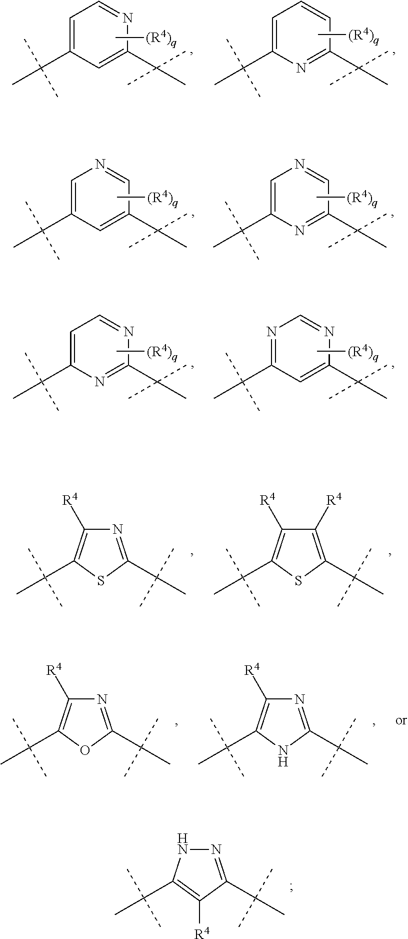

W is —N(R⁷)C(O)—, —C(O)N(R⁷)—, or a direct bond;
p is 0, 1, 2, 3, or 4;
q is 1, 2, or 3;
R¹ is hydrogen, alkyl, alkenyl, alkynyl, alkoxy, hydroxyalkyl, alkoxyalkyl, cycloalkyl, cycloalkylalkyl aryl, haloalkyl, aralkyl, heterocyclyl, heterocyclylalkyl, heteroaryl, or heteroarylalkyl;
R⁴ is independently alkyl, alkoxy, hydroxyalkyl, alkoxyalkyl, cycloalkyl, halo, trifluoromethyl, trifluoromethoxy, cyano, hydroxy, or —N(R⁷)₂
R⁶ is independently C₁-C₄alkyl, halo, trifluoromethyl, trifluoromethoxy, cyano, hydroxy, or —N(R⁷)₂; and
R⁷ is independently hydrogen, or C₁-C₄alkyl or
a pharmaceutically acceptable salt thereof or a prodrug thereof.

A subgroup of R¹ for the compounds of the invention is represented by Formula (III), wherein R¹ is hydrogen, C₁-C₄alkyl, C₂-C₆alkenyl, C₂-C₆alkynyl, C₁-C₆alkoxy, hydroxylC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, C₆-C₁₀arC₁-C₄alkyl, C₁-C₁₀heterocyclyl, C₁-C₁₀heterocyclylC₁-C₄alkyl, C₁-C₁₀heteroaryl, or C₁-C₁₀heteroarylC₁-C₄alkyl.

A subgroup of Q for the compounds of the invention is represented by Formula wherein W is

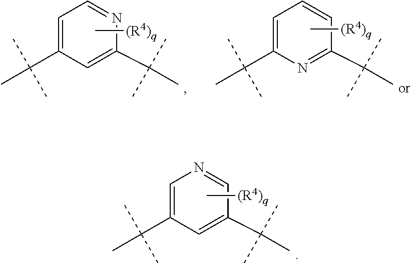

A subgroup of Q for the compounds of the invention is represented by Formula (II), wherein Q is

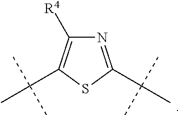

A subgroup of R⁴ for the compounds of the invention is represented by Formula (II), wherein R⁴ is C₁-C₄alkyl, C₁-C₆alkoxy, hydroxyC₁-C₄alkyl, C₁-C₆alkoxyC₁-C₄alkyl, C₃-C₇cycloalkyl, halo, trifluoromethyl, trifluoromethoxy, cyano, hydroxy, or —N(R⁷)₂.

In another embodiment the invention provides compounds represented by Formula

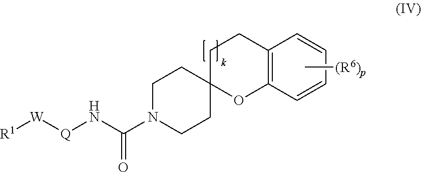

wherein Q is

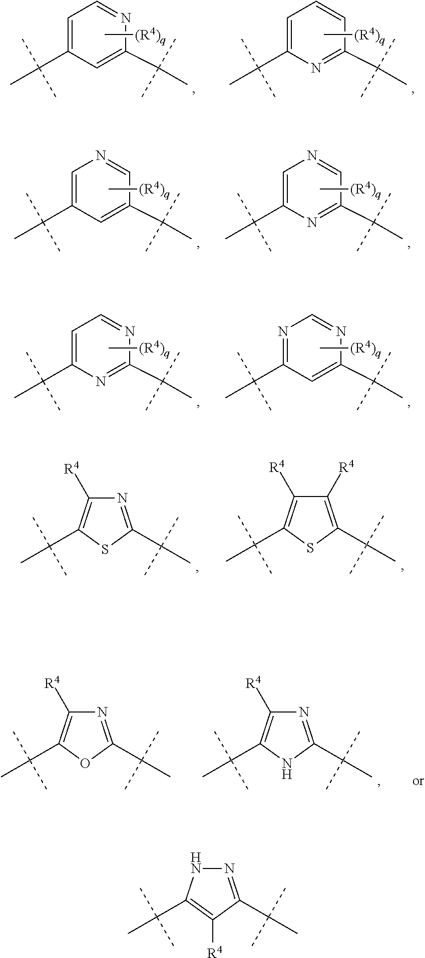

W is N(R⁷)C(O)—, —C(O)N(R⁷)—, or a direct bond;
k is 0 or 1;
p is 0, 1, 2, 3, or 4;
q is 1, 2 or 3;
R¹ is hydrogen, alkyl, alkenyl, alkynyl, alkoxy, hydroxyalkyl, alkoxyalkyl, cycloalkyl, cycloalkylalkyl, aryl, haloalkyl, aralkyl, heterocyclyl, heterocyclylalkyl, heteroaryl, or heteroarylalkyl;
R⁴ is independently alkyl, alkoxy, hydroxyalkyl, alkoxyalkyl, cycloalkyl, halo, trifluoromethyl, trifluoromethoxy, cyano, hydroxy, or —N(R⁷)₂;
R⁶ is independently C₁-C₄alkyl, halo, trifluoromethyl, trifluoromethoxy, cyano, hydroxy, or —N(R)₂; and
R⁷ is independently hydrogen, or C₁-C₄alkyl; or
a pharmaceutically acceptable salt thereof or a prodrug thereof.

A subgroup of R¹ for the compounds of the invention is represented by Formula (IV), wherein R¹ is hydrogen, C₁-C₄alkyl, C₂-C₆alkenyl, C₂-C₆alkynyl, C₁-C₆alkoxy, hydroxylC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, C₆-C₁₀arC₁-C₄alkyl, C₁-C₁₀heterocyclyl, C₁-C₁₀heterocyclylC₁-C₄alkyl, C₁-C₁₀heteroaryl, or C₁-C₁₀heteroarylC₁-C₄alkyl.

A subgroup of Q for the compounds of the invention is represented by Formula (IV), wherein Q is

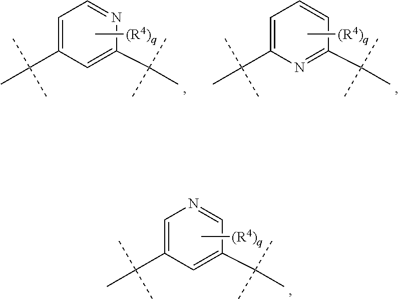

A subgroup of Q for the compounds of the invention is represented by Formula (IV), wherein Q is

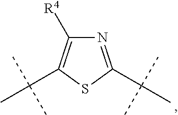

A subgroup of R⁴ for the compounds of the invention is represented by Formula (IV), wherein R⁴ is C₁-C₄alkyl, C₁-C₆alkoxy, hydroxylC₁-C₄alkyl, C₁-C₆alkoxyC₁-C₄alkyl, C₃-C₇cycloalkyl, halo, trifluoromethyl, trifluoromethoxy, cyano, hydroxy, or —N(R⁷)₂.

In another embodiment the invention provides compounds represented by Formula (V):

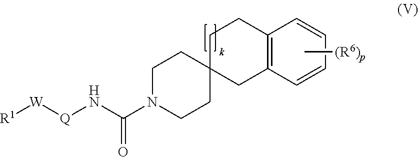

wherein Q is

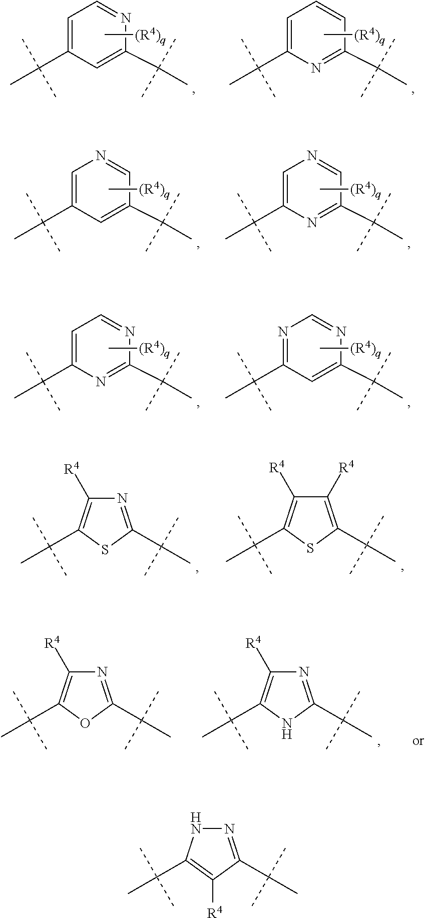

W is —N(R⁷)C(O)—, —C(O)N(R⁷)—, or a direct bond;
k is 0 or 1;
p is 0, 1, 2, 3, or 4;
q is 1, 2, or 3;
R¹ is hydrogen, alkyl, alkenyl, alkynyl, alkoxy, hydroxyalkyl, alkoxyalkyl, cycloalkyl, cycloalkylalkyl, aryl, haloalkyl, aralkyl, heterocyclyl, heterocyclylalkyl, heteroaryl, or heteroarylalkyl;
R⁴ is independently alkyl, alkoxy, hydroxyalkyl, alkoxyalkyl, cycloalkyl, halo, trifluoromethyl, trifluoromethoxy, cyano, hydroxy, or —N(R⁷)₂;
R⁶ is independently C₁-C₄alkyl, halo, trifluoromethyl, trifluoromethoxy, cyano hydroxy, or —N(R⁷)₂; and
R⁷ is independently hydrogen, or C₁-C₄alkyl; or
a pharmaceutically acceptable salt thereof or a prodrug thereof.

A subgroup of R¹ for the compounds of the invention is represented by Formula (V), wherein R¹ is hydrogen, C₁-C₄alkyl, C₂-C₆alkenyl, C₂-C₆alkynyl C₁-C₆alkoxy, hydroxylC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, C₆-C₁₀arC₁-C₄alkyl, C₁-C₁₀heterocyclyl, C₁-C₁₀heterocyclylC₁-C₄alkyl, C₁-C₁₀heteroaryl, or C₁-C₁₀heteroarylC₁-C₄alkyl.

A subgroup of Q for the compounds of the invention is represented by Formula (V), wherein Q is

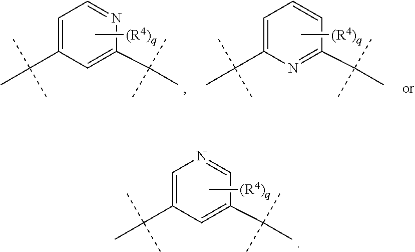

A subgroup of Q for the compounds of the invention is represented by Formula (V), wherein Q is

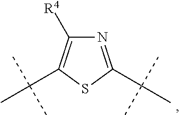

A subgroup of R⁴ for the compounds of the invention is represented by Formula (V), wherein R⁴ is C₁-C₄alkyl, C₁-C₆alkoxy, hydroxyC₁-C₄alkyl, C₁-C₆alkoxyC₁-C₄alkyl, C₃-C₇cycloalkyl, halo, trifluoromethyl, trifluoromethoxy, cyano, hydroxy, or —N(R⁷)₂.

In another embodiment the invention provides compounds according to Formula (I), (II), (III), (IV), or (V), wherein R¹ is alkyl, cyclocalkylalkyl, aralkyl or heteroarylalkyl, wherein the cycloalkylalkyl is

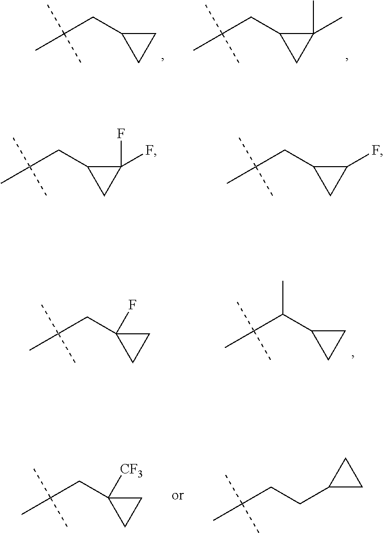

wherein the aralkyl is

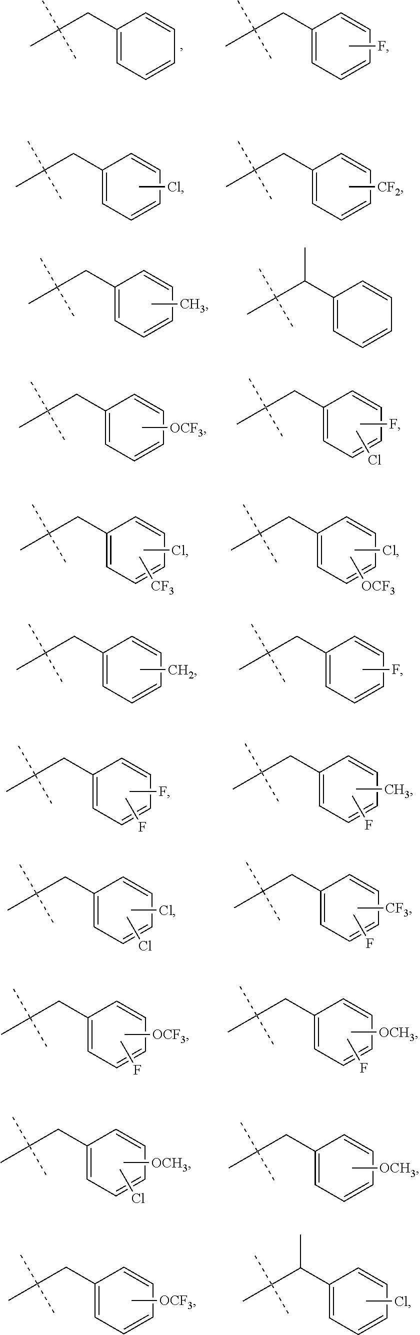

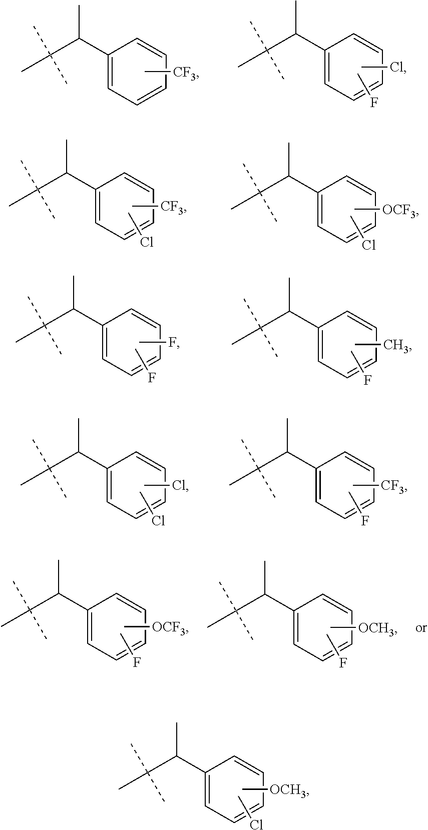

wherein the C₁-C₄ alkyl is methyl,

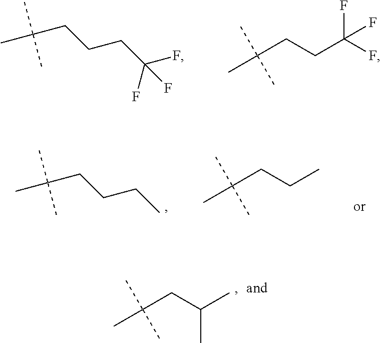

ethyl,
wherein the heteroarylalkyl is

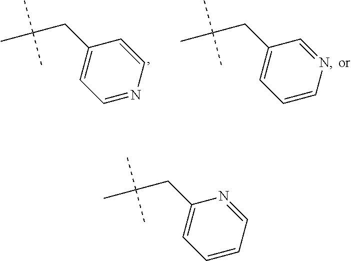

In another embodiment the invention provides compounds according to Formula (I), (a), (IV), or (V), wherein.

R¹ is C₁₋₄ alkyl, cyclocalkylalkyl, aralkyl or heteroarylalkyl,
wherein the cycloalkylalkyl is selected from the group consisting of

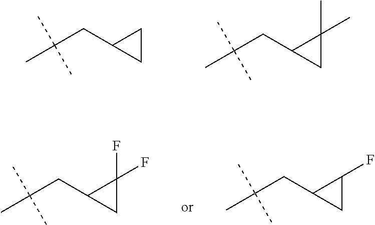

wherein the aralkyl is selected from the group consisting of

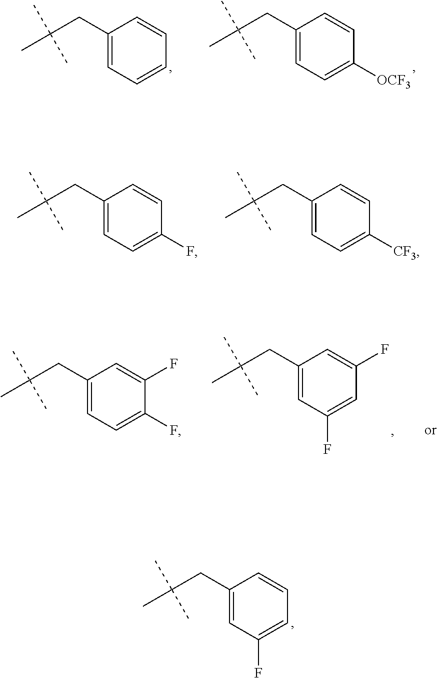

wherein the C₁-C₄ alkyl is selected from the group consisting of

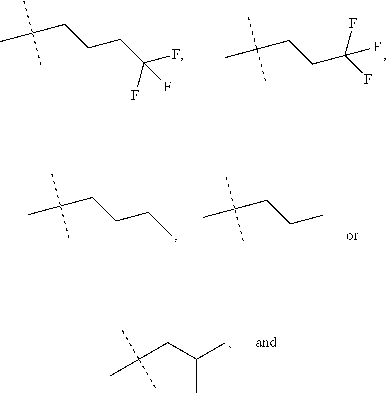

wherein the heteroarylalkyl is selected from the group consisting of

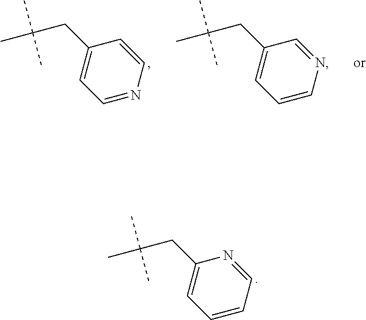

In another embodiment the invention provides compounds according to Formula (I), (H), (III), (IV), or (V), wherein

R¹ is hydrogen, methyl, (pyridin-2-yl)methyl, (5-methyl-isoxazol-3-yl)methyl, or (1-methyl-pyrazol-4-yl)methyl.

In another embodiment the invention provides compounds according to Formula (I), (II), (III), (IV), or (V), wherein

### W is —N(R⁷)C(O)—, and

R¹ is hydrogen,

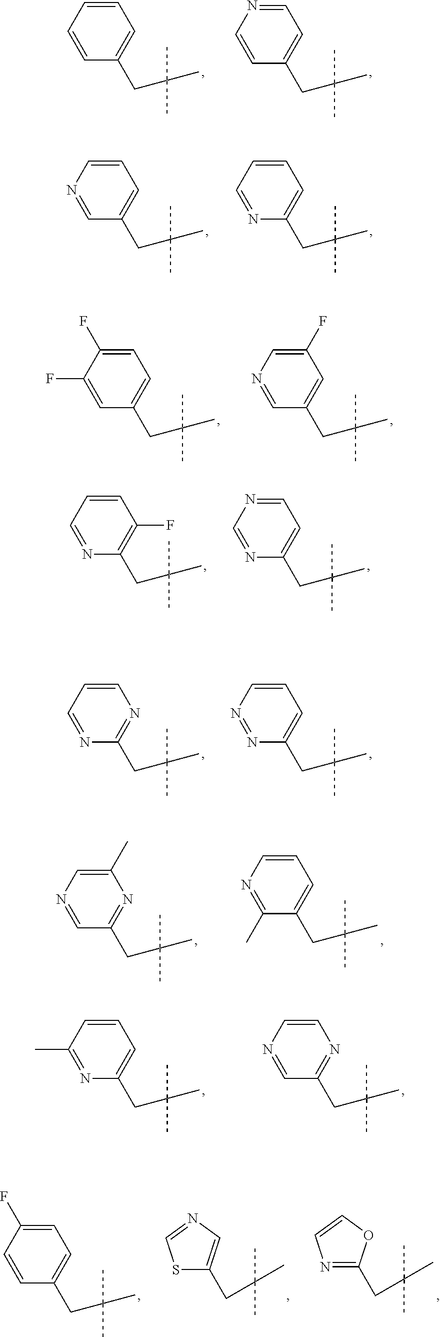

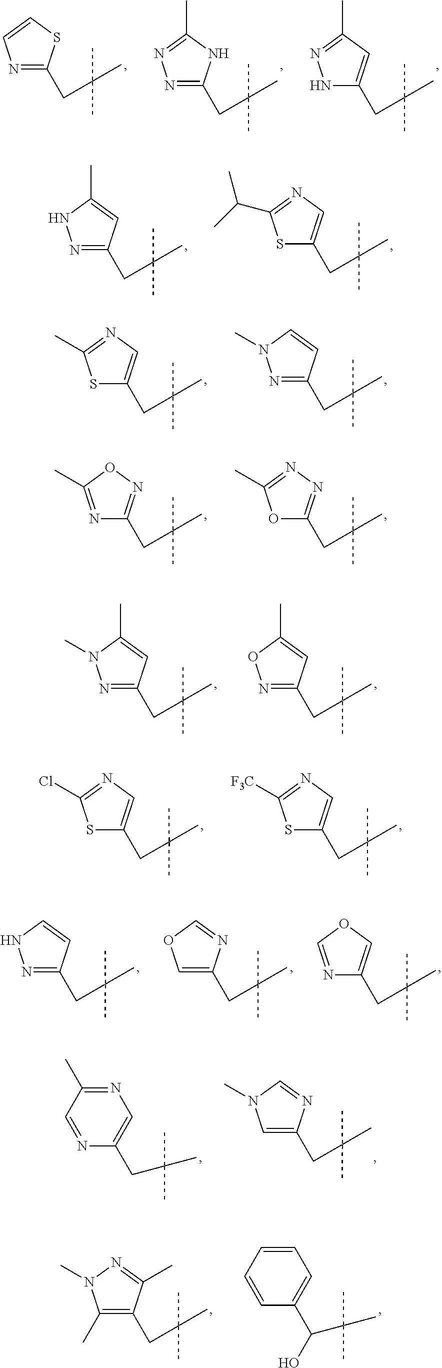

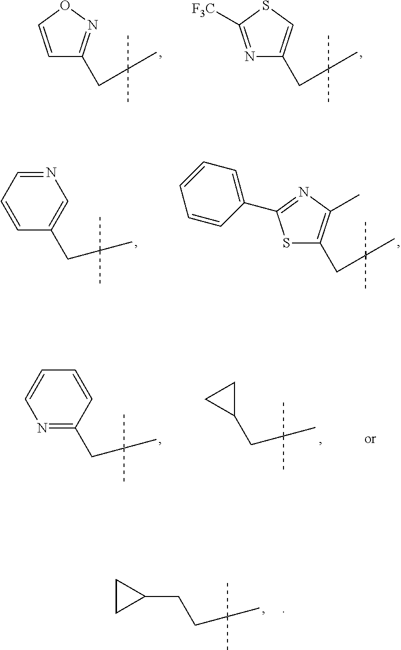

In another embodiment the invention provides compounds according to Formula (I), (II), (III), (IV), or (V), wherein

W is a direct bond and R¹ is

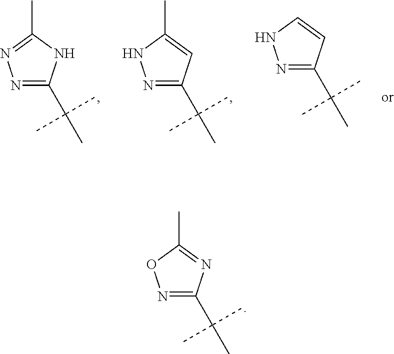

In another embodiment the invention provides according to Formula (I), (II), (II), (IV), or (V), wherein W is —N(R⁷)C(O)— and R¹ is methyl.

In another embodiment the invention provides compounds selected from the group consisting of
7-fluoro-N-(4-(methylcarbamoyl)pyridin-2-yl)-4-oxospiro[chroman-2,4′-piperidine]-1′-carboxamide;
7-fluoro-N-(4-(methylcarbamoyl)pyridin-2-yl)spiro[chroman-2,4′-piperidine]-1′-carboxamide;
8-chloro-N-(4-(methylcarbamoyl)pyridin-2-yl)spiro[chroman-2,4′-piperidine]-1′-carboxamide;
8-chloro-N-(4-(methylcarbamoyl)pyridin-2-yl)-4-oxospiro[chroman-2,4′-piperidine]-1′-carboxamide;
6-fluoro-N-(4-(methylcarbamoyl)pyridin-2-yl)spiro[chroman-2,4′-piperidine]-1′-carboxamide;
7-fluoro-N-(6-(methylcarbamoyl)pyridin-2-yl)spiro[chroman-2,4′-piperidine]-1′-carboxamide;
7-fluoro-N-(5-(methylcarbamoyl)pyridin-3-yl)spiro[chroman-2,4′-piperidine]-1′-carboxamide;
N-(4-(methylcarbamoyl)pyridin-2-yl)-1,3-dihydrospiro[indene-2,4′-piperidine]-1′-carboxamide;
N-(4-(methylcarbamoyl)pyridin-2-yl)spiro[chroman-2,4′-piperidine]1′-carboxamide;
7-fluoro-N-(4-(methylcarbamoyl)pyridin-2-yl)-3,4-dihydro-1H-spiro[naphthalene-2,4′-piperidine]-1′-carboxamide;
8-chloro-N-(4-(methylcarbamoyl)pyridin-2-yl)-3,4-dihydro-1H-spiro[naphthalene-2,4′-piperidine]-1′-carboxamide;
6-fluoro-N-(4-(methylcarbamoyl)pyridin-yl)-3,4-dihydro-1H-spiro[naphthalene-2,4′-piperidine]-1′-carboxamide;
N-(4-methylcarbamoyl)pyridin-2-yl)-3,4-dihydro-1H-spiro[naphthalene-2,4′-piperidine]-1′-carboxamide;
6-fluoro-N-(4-(methylcarbamoyl)pyridin-2-yl)-3H-spiro[benzofuran-2,4′-piperidine]-1′-carboxamide;
7-chloro-N-(4-(methylcarbamoyl)pyridin-2-yl)-3H-spiro[benzofuran-2,4′-piperidine]-1′-carboxamide;
5-fluoro-N-(4-(methylcarbamoyl)pyridin-2-yl)-3H-spiro[benzofuran-2,4′-piperidine]-1′-carboxamide;
N-(4-(methylcarbamoyl)pyridin-2-yl)-3H-spiro[benzofuran-2,4′-piperidine]-1′-carboxamide;
4-chloro-N-(4-(methylcarbamoyl)pyridin-2-yl)-1,3-dihydrospiro[indene-2,4′-piperidine]-1′-carboxamide;
5-fluoro-N-(4-(methylcarbamoyl)pyridin-2-yl)-1,3-dihydrospiro[indene-2,4′-piperidine]-1′-carboxamide;
6-fluoro-N-(4-{[(5-methyl-1,2-oxazol-3-yl)methyl]carbamoyl}pyridin-2-yl)-3,4-dihydro-1′H-spiro[chromene-2,4′-piperidine]-1′-carboxamide;
6-fluoro-N-(4-{[(1-methyl-1H-pyrazol-4-yl)methyl]carbamoyl}pyridin-2-yl)-3,4-dihydro-1′H-spiro[chromene-2,4′-piperidine]-1′-carboxamide;
N-(4-carbamoylpyridin-2-yl)-6-fluorospiro[chroman-2,4′-piperidine]1′-carboxamide; and
7-fluoro-N-(4-methyl-5-(pyridin-2-ylmethylcarbamoyl)thiazol-2-yl)spiro[chroman-2,4′-piperidine]-1′-carboxamide; or
   a pharmaceutically acceptable salt thereof or a prodrug thereof.

In one embodiment, the methods of the invention are directed towards the treatment and/or prevention of diseases mediated by stearoyl-CoA desaturase (SCD), especially human SCD (hSCD), preferably diseases related to dyslipidemia and disorders of lipid metabolism, and especially a disease related to elevated plasma lipid levels, cardiovascular disease, diabetes, obesity, metabolic syndrome, dermatological disorders, and the like by administering an effective amount of a compound of the invention.

The present invention also relates to pharmaceutical composition containing the compounds of the invention. In one embodiment, the invention relates to a composition comprising compounds of the invention in a pharmaceutically acceptable carrier and in an amount effective to modulate triglyceride level or to treat diseases related to dyslipidemia and disorders of lipid metabolism, when administered to an animal, preferably a mammal, most preferably a human patient. In an embodiment of such composition, the patient has an elevated lipid level, such as elevated triglycerides or cholesterol, before administration of said compound of the invention and the compound of the invention is present in an amount effective to reduce said lipid level.

### Utility and Testing of the Compounds of the Invention

The present invention relates to compounds, pharmaceutical compositions and methods of using the compounds and pharmaceutical compositions for the treatment and/or prevention of diseases mediated by stearoyl-CoA desaturase (SCD), especially human SCD (hSCD), preferably diseases related to dyslipidemia and disorders of lipid metabolism and especially a disease related to elevated plasma lipid levels, especially cardiovascular disease, diabetes, obesity, metabolic syndrome, dermatological disorders, and the like by administering to a patient in need of such treatment an effective amount of an SCD modulating, especially inhibiting, agent.

In general, the present invention provides a method for treating a patient for, or protecting a patient from developing, a disease related to dyslipidemia and/or a disorder of lipid metabolism, wherein lipid levels in an animal, especially a human being, are outside the normal range (i.e., abnormal lipid level, such as elevated plasma lipid levels), especially levels higher than normal, preferably where said lipid is a fatty acid, such as a free or complexed fatty acid, triglycerides, phospholipids, or cholesterol, such as where LDL-cholesterol levels are elevated or HDL-cholesterol levels are reduced, or any combination of these, where said lipid-related condition or disease is an SCD-mediated disease or condition, comprising administering to an animal, such as a mammal, especially a human patient, a therapeutically effective amount of a compound of the invention or a pharmaceutical composition comprising a compound of the invention wherein the compound modulates the activity of SCD, preferably human SCD1.

The compounds of the invention modulate, preferably inhibit, the activity of human SCD enzymes, especially human SCD1.

The general value of the compounds of the invention in modulating, especially inhibiting, the activity of SCD can be determined using the assay described below in Example 2.

Alternatively, the general value of the compounds in treating disorders and diseases may be established in industry standard animal models for demonstrating the efficacy of compounds in treating obesity, diabetes or elevated triglyceride or cholesterol levels or for improving glucose tolerance. Such models include Zucker obese fans rats (available from Harlan Sprague Dawley, Inc. (Indianapolis, Ind.)), or the Zucker diabetic fatty rat (ZDF/GmiCrl-fa/fa) (available from Charles River Laboratories (Montreal, Quebec)), and Sprague Dawley rats (Charles Rivers), as used in models for diet-induced obesity (Ghibaudi, L. et al. (2002). Obes. Res. Vol. 10, pp. 956-963). Similar models have also been developed for mice and Lewis rat.

The compounds of the instant invention are inhibitors of delta-9 desaturases and are useful for treating diseases and disorders in humans and other organisms, including all those human diseases and disorders which are the result of aberrant delta-9 desaturase biological activity or which may be ameliorated by modulation of delta-9 desaturase biological activity.

As defined herein, an SCD-mediated disease or condition is defined as any disease or condition in which the activity of SCD is elevated and/or where inhibition of SCD activity can be demonstrated to bring about symptomatic improvements for the individual so treated. As defined herein, an SCD-mediated disease or condition includes, but is not limited to, a disease or condition which is, or is related to, cardiovascular disease, dyslipidemias (including but not limited to disorders of serum levels of triglycerides, hypertriglyceridemia, VLDL, HDL, LDL, fatty acid Desaturation Index (e.g. the ratio of 18:1/18:0 fatty acids, or other fatty acids, as defined elsewhere herein), cholesterol, and total cholesterol, hypercholesterolemia, as well as cholesterol disorders (including disorders characterized by defective reverse cholesterol transport)), familial combined hyperlipidemia, coronary artery disease, arteriosclerosis, atherosclerosis, heart disease, cerebrovascular disease (including but not limited to stroke, ischemic stroke and transient ischemic attack (TIA)), peripheral vascular disease, and ischemic retinopathy.

An SCD-mediated disease or condition also includes metabolic syndrome (including but not limited to dyslipidemia, obesity and insulin resistance, hypertension, microalbuminemia, hyperuricaemia, and hypercoagulability), Syndrome X, diabetes, insulin resistance, decreased glucose tolerance, non-insulin-dependent diabetes mellitus, Type II diabetes, Type I diabetes, diabetic complications, body weight disorders (including but not limited to obesity, overweight, bulimia, cachexia and anorexia), weight loss, wasting disorders, body mass index and leptin-related diseases. In a preferred embodiment, compounds of the invention will be used to treat diabetes mellitus and/or obesity.

An SCD-mediated disease also includes obesity related syndromes, disorders and diseases that include, but not limited to obesity as a result of (i) genetics, (ii) diet (iii) food intake volume, (iv) a metabolic disorder, (v) a hypothalamic disorder, (vi) age, (vii) abnormal adipose distribution, (viii) abnormal adipose compartment distribution, (ix) compulsive eating disorders, and (x) motivational disorders which include the desire to consume sugars, carbohydrates, alcohols or drugs. Symptoms associates with obesity related syndromes, disorders and diseases include, but not limited to reduced activity. Obesity also increases the likelihood of sleep apnea, gallstones, osteoporosis and certain cancers.

As used herein, the term “metabolic syndrome” is a recognized clinical term used to describe a condition comprising combinations of Type II diabetes, impaired glucose tolerance, insulin resistance, hypertension, obesity, increased abdominal girth, hypertriglyceridemia, low HDL, hyperuricaernia, hypercoagulability and/or microalbuminemia. The American Heart Association has published guidelines for the diagnosis of metabolic syndrome, Grundy, S., et. al., (2006)Cardiol. Rev. Vol. 13, No. 6, pp, 322-327.

An SCD-mediated disease or condition also includes fatty liver, hepatic steatosis, vascular restenosis, hepatitis, non-alcoholic hepatitis, non-alcoholic steatohepatitis (NASH), alcoholic hepatitis, acute fatty liver, fatty liver of pregnancy, drug-induced hepatitis, erythrohepatic protoporphyria, iron overload disorders, hereditary hemochromatosis, hepatic fibrosis, hepatic cirrhosis, hepatoma and conditions related thereto.

An SCD-mediated disease or condition also includes binary cholesterol crystallization and related conditions, such as but not limited to gallstones, primary sclerosing cholangitis (PSC), progressive familial intrahepatic cholestasis (PFIC), high serum gamma-glutamyl transferase (GGT) PFIC, low-GGT PFIC (i.e. Byler disease, Byler syndrome), Caroli's disease, biliary helminthiasis, biliary strictures, choledocholithiasis, obstructive cholestasis, chronic cholestatic disease, presence of biliary sludge, and cholesterolosis of gallbladder.

An SCD-mediated disease or condition also includes but is not limited to a disease or condition which is or is related to primary hypertriglyceridemia, or hypertriglyceridemia secondary to another disorder or disease, such as hyperlipoproteinemias, familial histiocytic reticulosis lipoprotein lipase deficiency, apolipoprotein deficiency (such as ApoCII deficiency or ApoE deficiency), and the like, or hypertriglyceridemia of unknown or unspecified etiology.

An SCD-mediated disease or condition also includes a disorder of polyunsaturated fatty acid (PUFA) disorder, or a dermatological or skin disorder, including but not limited to eczema, acne, rosacea, skin ageing, seborrheic skin, psoriasis, keloid scar formation or prevention, diseases related to production or secretions from mucous membranes, such as monounsaturated fatty acids, wax esters, and the like. Preferably, the compounds of the invention will prevent or attenuate keloid scar formation by reduction of excessive sebum production that typically results in their formation. The investigation of the role of SCD inhibitors in the treatment of acne was advanced by the discovery that rodents lacking a functional SCD1 gene had changes to the condition of their eyes, skin, coat (Zheng Y., et al, “SCD1 is expressed in sebaceous glands and is disrupted in the asebia mouse”, Nat. Genet (1999) 23:268-270. Miyazaki, M., “Targeted Disruption of Stearoyl-CoA Desaturase1 Gene in Mice Causes Atrophy of Sebaceous and Meibomian Glands and Depletion of Wax Esters in the Eyelid”, J. Nutr. (2001), Vol. 131 pp 2260-68., Binczek, E. et al., “Obesity resistance of the stearoyl-CoA desaturase-deficient mouse results from disruption of the epidermal lipid barrier and adaptive thermoregulation”, Biol. Chem. (2007) Vol. 388 No. 4, pp 405-18).

An SCD-mediated disease or condition also includes inflammation, sinusitis, asthma, bronchitis, pancreatitis, osteoarthritis, rheumatoid arthritis, cystic fibrosis, and premenstrual syndrome.

An SCD-mediated disease or condition also includes but is not limited to a disease or condition which is or is related to cancer, polycystic ovary syndrome, neoplasia, malignancy, metastases, tumours (benign or malignant), carcinogenesis, hepatomas and the like.

An SCD-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry, including for administration to bovine, porcine or avian domestic animals or any other animal to reduce triglyceride production and/or provide leaner meat products and/or healthier animals.

An SCD-mediated disease or condition also includes a disease or condition that is, or is related to neurological diseases, psychiatric disorders, multiple sclerosis, eye diseases, polycystic ovary syndrome, sleep-disordered (e.g. disturbances of breathing or circadian rhythm, dysomnia, insomnia, sleep apnea, and narcolepsy), abnormal alanine transferase levels, respiratory disorders and immune disorders.

An SCD-mediated disease or condition also includes neurological diseases, including mild cognitive impairment (MCI), cerebral amyloid angipathy (CAA), down syndrome (DS), depression, schizophrenia, obsessive-compulsive disorder, and biopolar disorder.

An SCD-mediated disease or condition also includes neurodegenerative diseases, including Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Downs syndrome.

An SCD-mediated disease or condition also includes a disease or condition which is, or is related to, viral diseases or infections including but not limited to all positive strand RNA viruses, coronaviruses, SARS virus, SARS-associated coronavirus, Togaviruses, Picornaviruses, Coxsackievirus, Yellow Fever virus, Flaviviridae, ALPHAVIRUS (TOGAVIRIDAE) including Rubella virus, Eastern equine encephalitis virus, Western equine encephalitis virus, Venezuelan equine encephalitis virus, Sindbis virus, Semliki forest virus, Chikungunya virus, O'nyong'nyong virus, Ross river virus, Mayaro virus, Alphaviruses; ASTROVIRDAE including Astrovirus, Human Astroviruses; CALICIVIRIDAE including Vesicular exanthema of swine virus, Norwalk virus, Calicivirus, Bovine calicivirus, Pig calcivirus, Hepatitis E; CORONAVIRIDAE including Coronavirus, SARS virus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Berne virus, Breda virus; FLAVIVIRIDAE including Hepatitis C virus, West Nile virus, Yellow Fever virus, St. Louis encephalitis virus, Dengue Group, Hepatitis G virus, Japanese B encephalitis virus, Murray Valley encephalitis virus, Central European tick-borne encephalitis virus, Far Eastern tick-borne encephalitis virus, Kyasanur forest virus, Louping ill virus, Powassan virus. Omsk hemorrhagic fever virus, Kumilinge virus, Absetarov anzalova hypr virus, ITheus virus, Rocio encephalitis virus, Langat virus, Pestivirus, Bovine viral diarrhea. Hog cholera virus, Rio Bravo Group, Tyuleniy Group, Ntaya Group, Uganda S Group, Modoc Group; PICORNAVIRIDAE including Coxsackie A virus, Rhinovirus, Hepatitis A virus, Encephalomyocarditis virus, Mengovirus, ME virus, Human poliovirus 1, Coxsackie B POCYVIRIDAE including Potyvirus, Rymovirus, Bymovirus. Additionally it can be a disease or infection caused by or linked to Hepatitis viruses, Hepatitis B virus, Hepatitis C virus, human immunodeficiency virus (HIV) and the like. Treatable viral infections include those where the virus employs an RNA intermediate as part of the replicative cycle (hepatitis or HIV); additionally it can be a disease or infection caused by or linked to RNA negative strand viruses such as influenza and parainfluenza viruses.

The compounds identified in the instant specification inhibit the desaturation of various fatty acids (such as the C₉-C₁₀) desaturation of stearoyl-CoA), which is accomplished by delta-9 desaturase, such as stearoyl-CoA desaturase 1 (SCD1). As such, these compounds inhibit the formation of various fatty acids and downstream metabolites thereof. This may lead to an accumulation of stearoyl-CoA or palmitoyl-CoA and other upstream precursors of various fatty acids; which may possibly result in a negative feedback loop causing an overall change in fatty acid metabolism. Any of these consequences may ultimately be responsible for the overall therapeutic benefit provided by these compounds.

Typically, a successful SCD inhibitory therapeutic agent will meet some or all of the following criteria. Oral availability should be at or above 20%. Animal model efficacy is less than about 20 mg/Kg, 2 mg/Kg, 1 mg/Kg, or 0.5 mg/Kg and the target human dose is between 10 and 250 mg/70 Kg, although doses outside of this range may be acceptable. (“mg/Kg” means milligrams of compound per kilogram of body mass of the subject to whom it is being administered). The required dosage should preferably be no more than about once or twice a day or at meal times. The therapeutic index for ratio of toxic dose to therapeutic dose) should be greater than 10. The IC₅₀ (“Inhibitory Concentration ˜50%”) is a measure of the amount of compound required to achieve 50% inhibition of SCD activity, over a specific time period, in an SCO biological activity assay. Any process for measuring the activity of SCO enzymes, preferably mouse or human SCO enzymes, may be utilized to assay the activity of the compounds useful in the methods of the invention in inhibiting said SCD activity. Compounds of the invention demonstrate an IC₅₀ (“Inhibitory Concentration of 50%”) in a 15 minute microsomal assay of preferably less than 10 mM, less than 5 μM, less than 2.5 μM, less than 1 μM, less than 750 nM, less than 500 nM, less than 250 nM, less than 100 nM, less than 50 nM, and most preferably less than 20 nM. Compounds of the invention may show reversible inhibition (i.e., competitive inhibition) and preferably do not inhibit other iron binding proteins.

The identification of compounds of the invention as SCD inhibitors was readily accomplished using the SCD enzyme and microsomal assay procedure described in Shanklin J. and Summerville C., Proc. Natl. Acad. Sci. USA (1991), Vol. 88, pp. 2510-2514. When tested in this assay, compounds of the invention had less than 50% remaining SCD activity at 10 μM concentration of the test compound, preferably less than 40% remaining SCO activity at 10 μM concentration of the test compound, more preferably less than 30% remaining SCD activity at 10 μM concentration of the test compound, and even more preferably less than 20% remaining SCD activity at 10 μM concentration of the test compound, thereby demonstrating that the compounds of the invention are potent inhibitors of SCO activity.

These results provide the basis for analysis of the structure-activity relationship (SAR) between test compounds and SCD. Certain-groups tend to provide more potent inhibitory compounds. SAR analysis is one of the tools those skilled in the art may employ to identify preferred embodiments of the compounds of the invention for use as therapeutic agents. Other methods of testing the compounds disclosed herein are also readily available to those skilled in the art. Thus, in addition, the determination of the ability of a compound to inhibit SCD may be accomplished in vivo. In one such embodiment this is accomplished by administering said chemical agent to an animal afflicted with a triglyceride (TG)- or very low density lipoprotein (VLDL)-related disorder and subsequently detecting a change in plasma triglyceride level in said animal thereby identifying a therapeutic agent useful in treating a triglyceride (-TG)- or very to density lipoprotein (VLDL)-related disorder. In such embodiment, the animal may be a human, such as a human patient afflicted with such a disorder and in need of treatment of said disorder.

In specific embodiments of such in vivo processes, said change in SCD1 activity in said animal is a decrease in activity, preferably wherein said SCD1 modulating agent does not substantially inhibit the biological activity of a delta-5 desaturase, delta-6 desaturase or fatty acid synthetase or other enzymes containing iron at the active site.

The model systems useful for compound evaluation may include, but are not limited to, the use of liver microsomes, such as from mice that have been maintained on a high carbohydrate diet, or from human donors, including persons suffering from obesity. Immortalized cell lines, such as HepG2 (from human liver), MCF-7 (from human breast cancer) and 3T3-L1 (from mouse adipocytes) may also be used Primary cell lines, such as mouse primary hepatocytes, are also useful in testing the compounds of the invention. Where whole animals are used, mice used as a source of primary hepatocyte cells may also be used wherein the mice have been maintained on a high carbohydrate diet to increase SCD activity in mirocrosomes and/or to elevate plasma triglyceride levels (i.e., the 18:1/18:0 ratio) alternatively mice on a normal diet or mice with normal triglyceride levels may be used. Mouse models employing transgenic mice designed for hypertriglyceridemia are also available. Rabbits and hamsters are also useful as animal models, especially those expressing CETP (cholesterol ester transfer protein).

Another suitable method for determining the in vivo efficacy of the compounds of the invention is to indirectly measure their impact on inhibition of SCD enzyme by measuring a subject's Desaturation Index after administration of the compound.

“Desaturation Index” as employed in this specification means the ratio of the product over the substrate for the SCD enzyme as measured from a given tissue sample. This may be calculated using three different equations 18:1n−9/18:0 (oleic acid over stearic acid); 16:1n−7/160 (palmitoleic acid over palmitic acid); and/or 16:1n−7+1811n−7/16:0 (measuring all reaction products of 16:0 desaturation over 16:0 substrate).

Desaturation Index is primarily measured in liver or plasma triglycerides, but may also be measured in other selected lipid fractions from a variety of tissues. Desaturation Index, generally speaking, is a tool for plasma lipid profiling.

A number of human diseases and disorders are the result of aberrant SCD1 biological activity and may be ameliorated by modulation of SCD1 biological activity using the therapeutic agents of the invention.

Inhibition of SCD expression may also affect the fatty acid composition of membrane phospholipids, as well as production or levels of triglycerides and cholesterol esters. The fatty acid composition of phospholipids ultimately determines membrane fluidity, with a subsequent modulation of the activity of multiple enzymes present within the membrane, while the effects on the composition of triglycerides and cholesterol esters can affect lipoprotein metabolism and adiposity.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented.

For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

Alternatively, another format can be used to measure the effect of SCD inhibition on sebaceous gland function. In a typical study using ridnets, oral, intravenous or topical formulations of the SCD inhibitor are administered to a rodent for a period of 1 to 8 days. Skin samples are taken and prepared for histological assessment to determine sebaceous gland number, size, or lipid content. A reduction of sebaceous gland size, number or function would indicate that the SCD inhibitor would have a beneficial impact on acne vulgaris, (Clark, S. B. et al “Pharmacological modulation of sebaceous gland activity: mechanisms and clinical applications”, Dermatol. Clin. (2007) Vol. 25. No. 2, pp 137-46. Geiger, J. M., “Retinoids and sebaceous gland activity” Dermatology (1995), Vol. 191, No. 4, pp 305-10),

### Pharmaceutical Compositions of the Invention and Administration

The present invention also relates to pharmaceutical composition containing the compounds of the invention disclosed herein, in one embodiment, the present invention relates to a composition comprising compounds of the invention in a pharmaceutically acceptable carrier and in an amount effective to modulate triglyceride level or to treat diseases related to dyslipidemia and disorders of lipid metabolism, when administered to an animal, preferably a mammal, most preferably a human patient. In an embodiment of such composition, the patient has an elevated lipid level, such as elevated triglycerides or cholesterol, before administration of said compound of the invention and the compound of the invention is present in an amount effective to reduce said lipid level.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub, Co., N.J. current edition).

Those skilled in the art are familiar with how to determine suitable doses of the compounds for use in treating the diseases and disorders contemplated herein.

Therapeutic doses are generally identified through a dose ranging study in humans based on preliminary evidence derived from animal studies. Doses must be sufficient to result in a desired therapeutic benefit without causing unwanted side effects for the patient. The preferred dosage range for an animal is 0.001 mg/Kg to 10,000 mg/Kg, including 0.5 mg/Kg, 1.0 mg/Kg, 2.0 mg/Kg, 5.0 mg/Kg, 10 mg/Kg and 20 mg/Kg, though doses outside this range may be acceptable. The dosing schedule may be once or twice per day, although more often or less often may be satisfactory.

Those skilled in the art are also familiar with determining administration methods (oral, intravenous, inhalation, sub-cutaneous, transdermal, topical, etc.), dosage forms, suitable pharmaceutical excipients and other matters relevant to the delivery of the compounds to a subject in need thereof.

In an alternative use of the invention, the compounds of the invention can be used in in vitro or in vivo studies as exemplary agents for comparative purposes to find other compounds also useful in treatment of, or protection from, the various diseases disclosed herein.

The pharmaceutical compositions according to the invention are those suitable for enteral, such as oral or rectal, transdermal, intravenous, intradermal, subcutanceous, intramuscular, colonical, ophthalmic, intraurethral, nasal (e.g. inhalation), intraperitoneal and parenteral administration to mammals, including man, to inhibit stearoyl-CoA desaturase, and for the treatment of conditions associated with stearoyl desaturase activity. In general, the pharmaceutical compositions comprise a therapeutically effective amount of a pharmacologically active compound of the instant invention, alone or in combination with one or more pharmaceutically acceptable carriers.

The pharmacologically active compounds of the invention are useful in the manufacture of pharmaceutical compositions comprising a therapeutically effective amount thereof in conjunction or admixture with excipients or carriers suitable for either enteral or parenteral application. For enteral or parenteral application, it is preferred to administer an effective amount of a pharmaceutical composition according to the invention as tablets or gelatin capsules. Such pharmaceutical compositions may comprise, for example, the active ingredient together with diluents (e.g. lactose, dextrose, sucrose, mannitol, sorbitol, cellulose and/or glycine), lubricants (e.g., silica, talcum, stearic acid, its magnesium or calcium salt and/or polyethyleneglycol), and for tablets also comprises binders (e.g., magnesium aluminum silicate, starch paste, gelatin, tragacanth, methylcellulose, sodium carboxymethylcellulose and/or polyvinylpyrrolidone) and disintegrants (e.g., starches, agar, alginic acid or its sodium salt) or effervescent mixtures and absorbents, colorants, flavors and sweeteners.

In another aspect of the present invention the compounds may be in the form of injectable compositions, e.g. preferably aqueous isotonic solutions or suspensions, and suppositories, which can be advantageously prepared from fatty emulsions or suspensions. The compositions may be sterilized and/or contain adjuvants, such as preserving, stabilizing wetting or emulsifying agents, solution promoters, salts for regulating the osmotic pressure and/or buffers. In addition, they may also contain other therapeutically valuable substances. The compositions may be prepared according to conventional mixing, granulating or coating methods, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate-controlling barrier to deliver the compound of the skin of the host at a controlled and pre-determined rate over a prolonged period of time and means to secure the device to the skin.

The most suitable route will depend on the nature and severity of the condition being treated. Those skilled in the art are also familiar with determining administration methods, dosage forms, suitable pharmaceutical excipients and other matters relevant to the delivery of the compounds to a subject in need thereof.

The compounds of the invention may be usefully combined with one or more other therapeutic agents for the treatment of SCD-mediated diseases and conditions. Preferably, the other therapeutic agent is selected from antidiabetics, hypolipidemic agents, anti-obesity agents, anti-hypertensive agents or inotropic agents.

Thus, an additional aspect of the present invention concerns a pharmaceutical composition comprising a therapeutically effective amount of a compound of the invention in combination with one or more other therapeutic agents. For example, the composition can be formulated to comprise a therapeutically effective amount of a compound of the invention as defined above, in combination with another therapeutic agent, each at an effective therapeutic dose as reported in the art. Such therapeutic agents may, for example, include insulin, insulin derivatives and mimetics; insulin secretagogues, such as the sulfonylureas, e.g., Glipizide, glyburide and Amaryl; insulinotropic sulfonylurea receptor ligands, such as meglitinides, e.g., nateglinide and repaglinide; PPARγ and/or PPARα (peroxisome proliferator-activated receptor) ligands such as MCC-555, MK767, L-165041, GW7282 or thiazolidinediones such as rosiglitazone, pioglitazone, balaglitazone, troglitazone and the like; insulin sensitizers, such as protein tyrosine phosphatase-1B (PIP-1B) inhibitors such as PTP-112; GSK3 (glycogen synthase kinase-3) inhibitors such as SB-517955, SB-4196052, SB-216763, NN-57-05441, N,N-57-05445 or RXR ligands such as GW-0791, AGN-194204; sodium-dependent glucose cotransporter inhibitors, such as T-1095, glycogen phosphorylase A inhibitors, such as BAY R3401; biguanides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors such as LAF237 (Vildagliptin) or sitagliptin; GIP and GIP mimetics such as those disclosed in WO 00/58360; PACAP and PACAP mimetics, such as those disclosed in WO 01/23420; hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (farnesoid X receptor) and LXR (liver X receptor) ligands, cholestyramine, fibrates, nicotinic acid and aspirin', anti-obesity agents, such as orlistat, anti-hypertensive agents, inotropic agents and hypolipidemic agents, e.g., loop diuretics, such as ethacrynic acid, furosemide and torsemide; angiotensin converting enzyme ACE) inhibitors, such as benazepril, captopril, enalapril, fosinopril, lisinopril, moexipril, perinodopril, quinapril, ramipril and trandolapril; inhibitors of the Na—K-ATPase membrane pump, such as digoxin; neutralendopeptidase (NEP) inhibitors; ACE/NEP inhibitors, such as omapatrilat, sampatrilat and fasidotril; angiotensin II antagonists, such as candesartan, eprosartan, irbesartan, losartan, telmisartan and valsartan, in particular valsartan; β-adrenergic receptor blockers, such as acebutolol, atenolol, betaxolol, bisoprolol, metoprolol, nadolol, propranolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil. Other specific antidiabetic compounds are described by Patel Mona (Expert Opin Investig Drugs. (2003) Apr; 12(4):623-33) in the FIGS. 1 to 7. A compound of the present invention may be administered either simultaneously, before or after the other active ingredient, either separately by the same or different route of administration or together in the same pharmaceutical formulation.

The structure of the active agents identified by code numbers (nos.), generic or trade names may be taken from the actual edition of the standard compendium “The Merck Index” or from databases, e.g. Patents international (e.g. IMS World Publications).

In another aspect is the use of the pharmaceutical composition as described above for production of a medicament for the treatment of SCD-mediated disease or conditions.

In another aspect is the use of a pharmaceutical composition or combination as described above for the preparation of a medicament for the treatment of conditions associated with stearoyl-CoA desatruase activity.

In another embodiment, the invention provides pharmaceutical compositions as described above for the treatment of conditions associated with the inhibition of stearoyl-CoA desaturase.

### Preparations of Compounds

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), methoxybenzyl, trityl and the like. Suitable protecting groups for carboxylic acid include alkyl, aryl or arylalkyl esters.

Protecting groups may be added or removed in accordance with standard techniques, which are well-known to those skilled in the art and as described herein. The use of protecting groups is described in detail in Green, T. W. and P. G. M. Wuts, Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives OF compounds of this invention may not possess pharmacological activity as such they may be administered to a mammal arid thereafter metabolized in the body to form compounds of the invention which are pharmacologically active. Such derivatives may therefore be described as “prodrugs”. All prodrugs of compounds of this invention are included within the scope of the invention.

The following reaction schemes illustrate methods to make compounds of this invention. It is understood that one skilled in the art would be able to make these compounds by similar methods or by methods known to one skilled in the art. In general, starting components may be obtained from sources such as Sigma Aldrich, Lancaster Synthesis, Inc Maybridge, Matrix Scientific, TCI, and Fluorochem USA, etc. or synthesized according to sources known to those skilled in the art (see, e.g., Advanced Organic Chemistry Reactions, Mechanisms, and Structure, 5th edition (Wiley, December 2000)) or prepared as described in this invention, R¹, R², R³, R⁴, R⁵, R⁶, R⁷, Q, p, and W are defined as in the Specification unless specifically defined otherwise, R′ is a protecting group.

In general, the compounds of Formula (I) of this invention where k is 1; m and n are each 0; R² is hydrogen; Z is —O—; and W is —N(R⁷)C(O)—; can be synthesized following the general procedure as described in REACTION SCHEME 1.

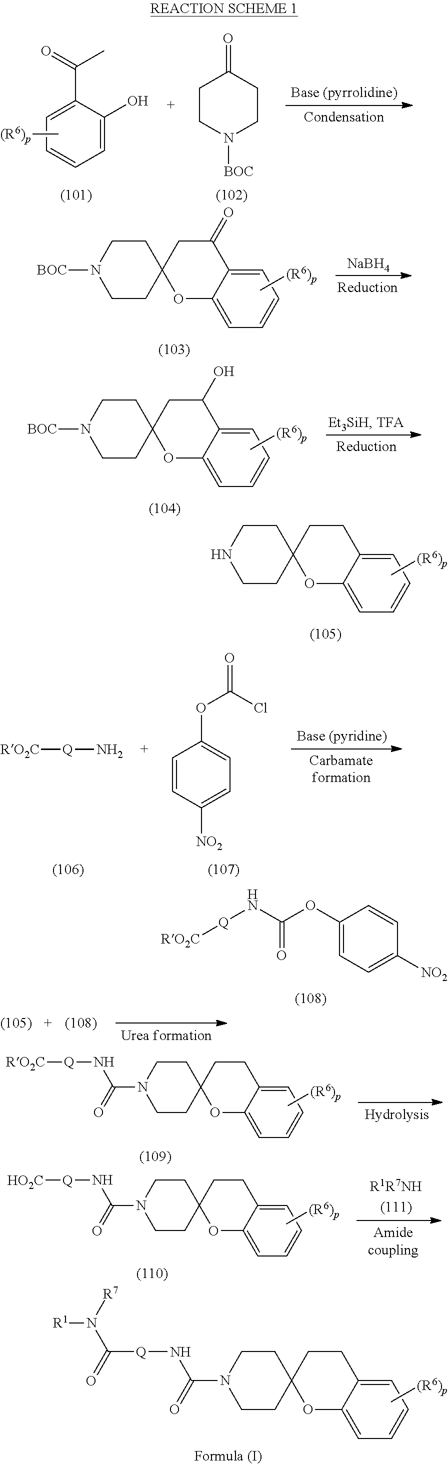

The starting materials for the above reaction scheme are commercially available or can be prepared according to methods known to one skilled in the art or by methods disclosed herein. In general, the compounds of the invention are prepared in the above reaction scheme as follows:

A phenol compound (101) reacts with oxopiperidine (102) in the presence of a base, such as pyrrolidine, to generate the oxospiro compound (103). Reduction of the carbonyl group of compound (103) by a reducing agent, such as but not limited to sodium borohydride, affords the hydroxy compound (104) which undergoes the further reduction in the presence of triethylsilane in trifluoroacetic acid to afford spiro compound (105). In parallel an amine compound (106) reacts with 4-nitrophenyl chloroformate (107) to generate carbamate compound (108) which readily reacts with the spiro compound (105) to afford the ester compound (109). Hydrolysis of the ester compound (109) under standard reaction conditions known to one skilled in the art affords the carboxylic acid (110). Amide bond formation between the carboxylic acid (110) and art amine compound (111) affords the compounds of Formula (I) of the invention where k is 1; m and n are 0; R² is hydrogen; Z is —O—; and W is —N(R⁷)C(O)—.

Alternatively, the compounds of Formula (I) of this invention where m is 0, k is 1; n is 2; R² is hydrogen; two R⁵'s on the carbon adjacent to the phenyl ring together form an oxo (═O); Z is —O—; and W is —N(R⁷)C(O)—, can be synthesized following the general procedure as described in REACTION SCHEME 2.

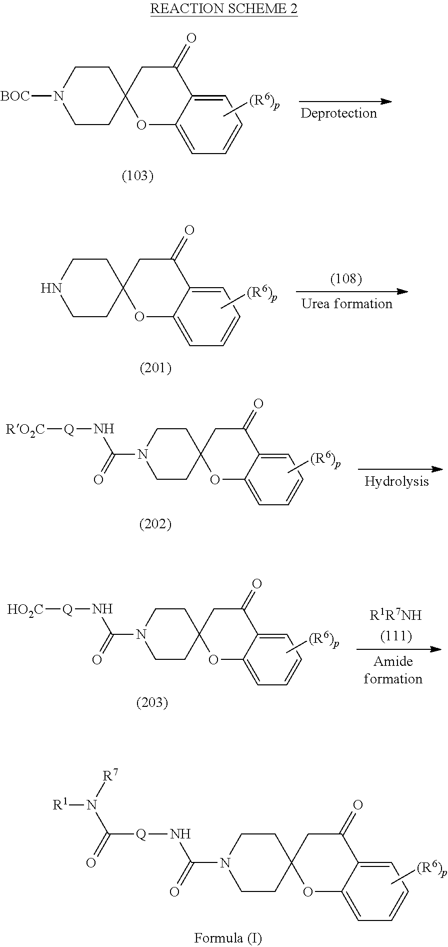

The starting materials for the above reaction scheme are commercially available or can be prepared according to methods known to one skilled in the art or by methods disclosed herein. In general, the compounds of the invention are prepared in the above reaction scheme as follows:

The oxospiro compound (103) is treated with an acid, such as, but not limited to, trifluoroacetic acid, to generate the compound (201). Compound (201) readily reacts with compound (108) to afford the ester compound (202). Hydrolysis of the ester compound (202) under standard reaction conditions known to one skilled in the art affords the carboxylic acid (203). Amide bond formation between the carboxylic acid (203) and an amine compound (III) affords the compounds of Formula (I) of the invention m is 0; k is 1; n is 2; R² is hydrogen; two R⁵'s on the carbon adjacent to the phenyl ring together form an oxo (O); Z is —O—; and W is —N(R⁷)C(O)—.

Alternatively, the compounds of Formula (I) of this invention where k is 1; m and n are each 0; R² is hydrogen; Z is —O—, and W is —N(R⁷)C(O)—; can be synthesized following the general procedure as described in REACTION SCHEME 3.

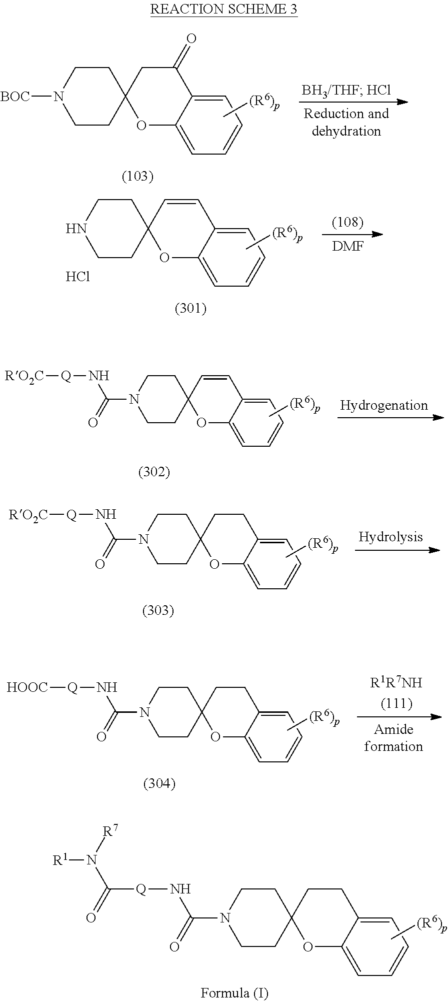

The starting materials for the above reaction scheme are commercially available or can be prepared according to methods known to one skilled in the art or by methods disclosed herein. In general, the compounds of the invention are prepared in the above reaction scheme as follows:

The oxospiro compound (103) is reduced with a reducing agent, such as but not limited to borane, followed by dehydration to generate the compound (301). Compound (301) readily reacts with compound (108) to afford compound (302). Hydrogenation of (302) under standard conditions affords compound (303). Hydrolysis of compound (303) under standard reaction conditions known to one skilled in the art affords carboxylic acid (304). Amide bond formation between carboxylic acid (304) and an amine compound (111) affords the compounds of Formula (I) of the invention where k is 1; m and n are each 0; R², R³ and R⁵ are hydrogen; Z is —O—, and W is —N(R⁷)C(O)—.

Alternatively, the compounds of Formula (I) of this invention where k is 1; m and n are each 0; R¹ is methyl; R² is hydrogen; Z is —C(H)₂—, W is —N(R⁷)C(O)—; and Q is

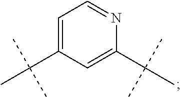

can be synthesized following the general procedure as described in REACTION SCHEME 4.

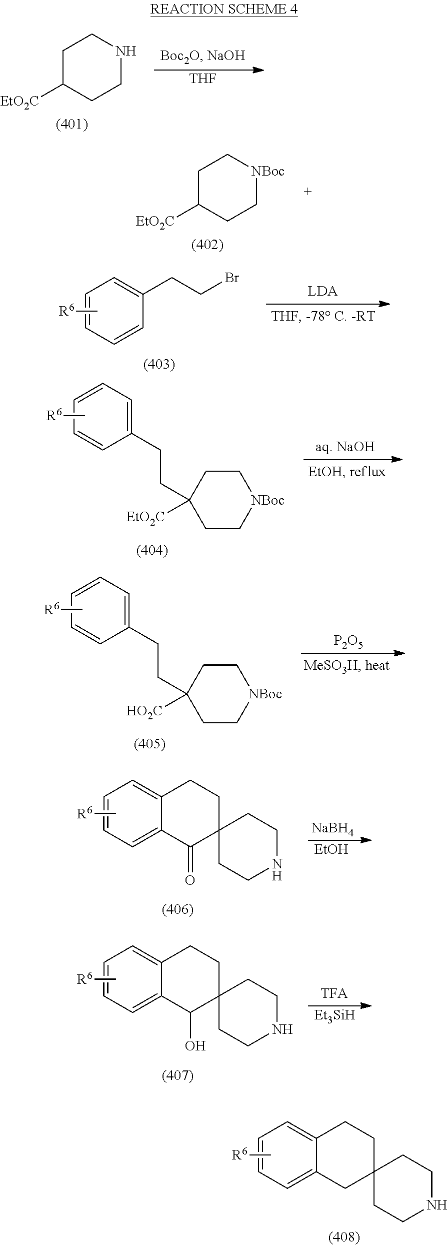

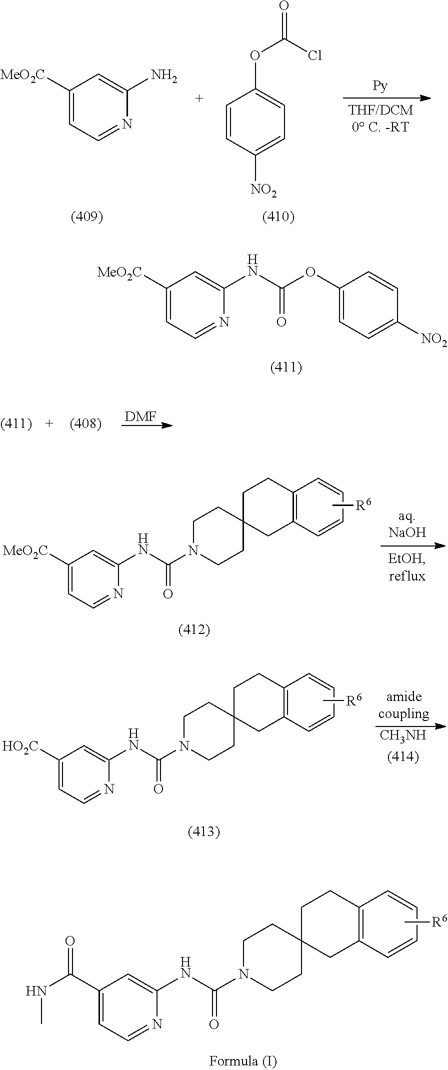

The starting materials for the above reaction scheme are commercially available or can be prepared according to methods known to one skilled in the art or by methods disclosed herein. In general, the compounds of the invention are prepared in the above reaction scheme as follows:

A piperidinyl compound (401) is protected with di-tert-butyl-dicarbonate (Boc₂O) in the presence of a solvent, such as, but not limited to tetrahydrofuran (THF) to give the carbamate compound (402). The carbamate compound (402) is then alkylated with a base, such as but not limited to lithium diisopropylamide (LDA), and halide compound (403) to afford the ester compound (404). Hydrolysis of the ester compound (404) under standard reaction conditions known to one skilled in the art affords the carboxylic acid (405). Treatment of the compound (405) with an acid, such as but not limited to phosphorus pentoxide-methanesulfonic acid (7% by weight), provides the spire compound (406). Reduction of the carbonyl group of compound (406) by a reducing agent, such as but not limited to sodium borohydride, affords the compound (407), which undergoes the further reduction in the presence of triethylsilane in trifluoroacetic acid to afford spiro compound (408). In parallel, methyl 2-aminoisonicotinate (409) reacts with 4-nitrophenyl chloroformate (410) to generate carbamate compound (411), which readily reacts with spiro compound (408) to afford the ester compound (412). Hydrolysis of the ester compound (412) under standard reaction conditions known to one skilled in the art affords the carboxylic acid (413). Amide bond formation between the carboxylic acid (413) and an amine compound (414) affords the compounds of Formula (I) of the invention where k is 1; m and n are each 0; R¹ is methyl; R² is hydrogen; Z is —C(H)₂—, W is —N(R⁷)C(O)—; and Q is

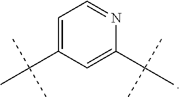

Alternatively, the compounds of Formula (I) of this invention where m, n and k are each 0; R¹ is methyl; R² and R⁷ are each hydrogen; Z is —O—, W is —N(R⁷)C(O)—; and Q is

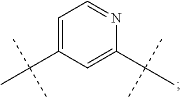

can be synthesized following the general procedure as described in REACTION SCHEME 5.

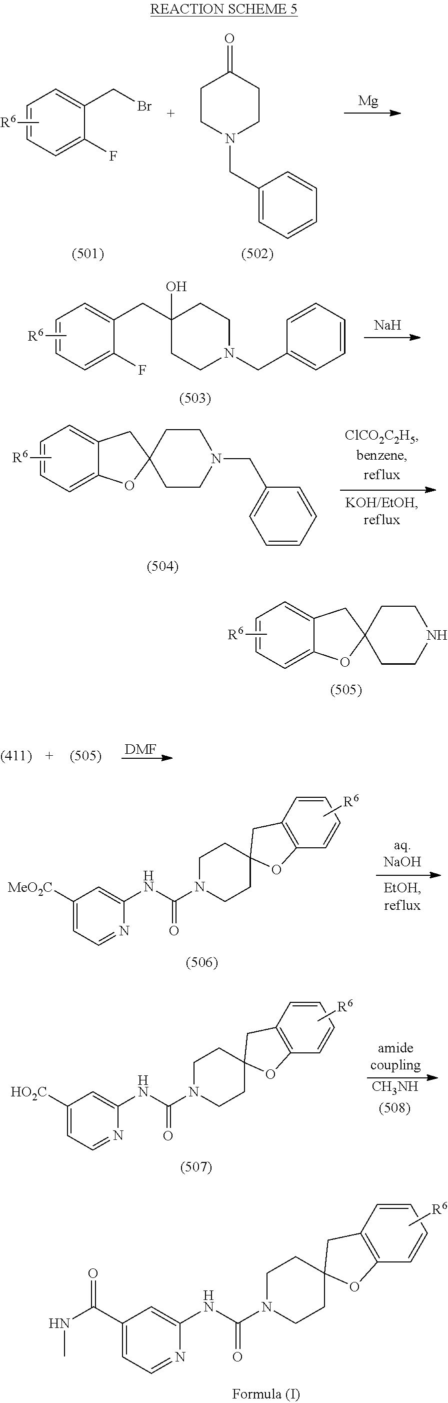

The starting materials for the above reaction scheme are commercially available or can be prepared according to methods known to one skilled in the art or by methods disclosed herein in general, the compounds of the invention are prepared in the above reaction scheme as follows:

The halide compound (501) is condensed via a Grignard reaction with 1-benzyl-4-piperidine (502) to form the piperidinol compound (503). Reaction of compound (503) with a base, such as but not limited to sodium hydride, in a solvent such as but not limited to benzene-dimethylformamide, under standard reflux conditions affords the spiro compound (504). Debenzylation of the spiro compound (504) under standard reaction conditions in the presence of ethyl chloroformate, which then readily undergoes hydrolysis under standard reaction conditions to afford the spiro compounds (506). The spiro compound (505) reacts with the carbamate compound (411) to afford the ester compound (506). Hydrolysis of the ester compound (506) under standard reaction conditions known to one skilled in the art affords the carboxylic acid (507). Amide bond formation between the carboxylic acid (507) and an amine compound (508) affords the compounds of Formula (I) of the invention where m n and k are each 0; R¹ is methyl; R², and R⁷ are each hydrogen; Z is —O—, W is —N(R⁷)C(O)—; and Q is

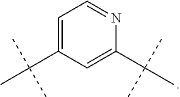

Alternatively, the compounds of Formula (I) of this invention where m n, and k are 0; p is 1; R¹ is methyl; R² is hydrogen; Z is —C(H)₂—, W is —N(R⁷)C(O)—; and Q is

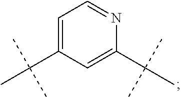

can be synthesized following the general procedure as described in REACTION SCHEME 6.

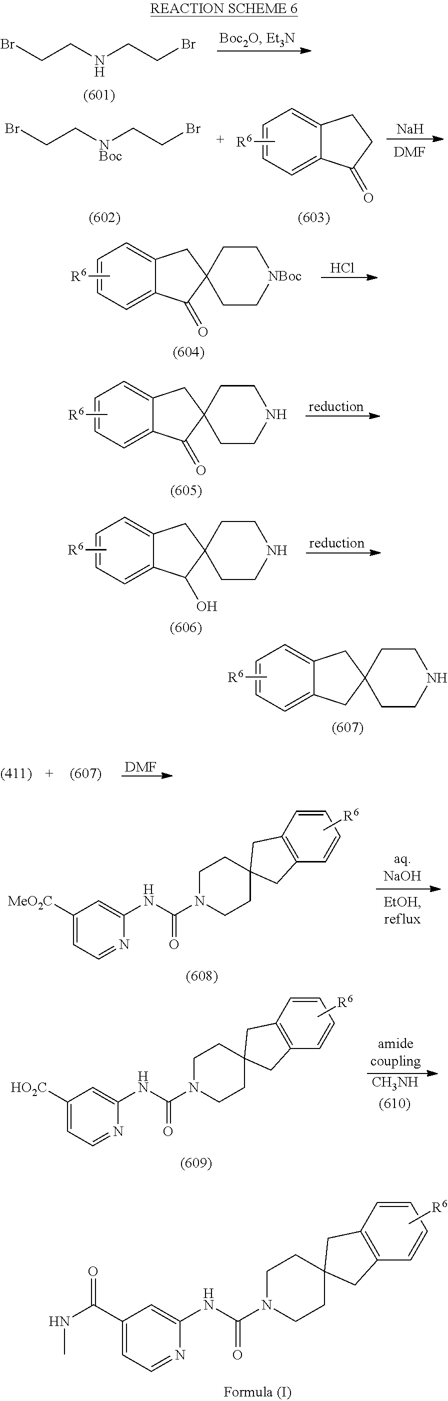

The starting materials for the above reaction scheme are commercially available or can be prepared according to methods known to one skilled in the art or by methods disclosed herein. In general, the compounds of the invention are prepared in the above reaction scheme as follows:

A bis(2-bromoethyl)amine compound (601) is protected with di-tert-butyl-dicarbonate (Boc₂O) in the presence of a solvent, such as but not limited to tetrahydrofuran (THF), to give the carbamate compound (602). The carbamate compound (602) reacts with 2,3-dihydroinden1-one (603) in the presence of a base, such as but not limited to sodium hydride, to afford the spiro compound (604). Removal of the BOC protecting group with an acid affords compound (606). Reduction of the carbonyl group of compound (605) by a reducing agent, such as but not limited to sodium borohydride, affords compound (606), which undergoes the further reduction in the presence of triethylsilane in trifluoroacetic acid to afford the spiro compound (607). The spiro compound (607) reacts with the carbamate compound (411) to afford the ester compound (608). Hydrolysis of the ester compound (608) under standard reaction conditions known to one skilled in the art affords the carboxylic acid (609). Amide bond formation between the carboxylic acid (609) and an amine compound (610) affords the compounds of Formula (I) of the invention where m, n, and k are 0; p is 1; R¹ is methyl; R² is hydrogen; Z is —C(H)ᵣ, W is —N(R⁷)C(O)—; and Q is

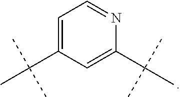

### Preparation 1

### Preparation of methyl 2-((4-nitrophenoxy)carbonylamino)isonicotinate

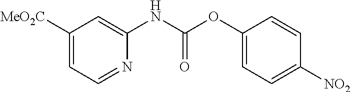

To a stirred mixture of methyl 2-aminopyridine-4-carboxylate (4.00 g, 26.29 mmol) and pyridine (3.20 mL, 39.56 mmol) in dichloromethane (80 mL) and tetrahydrofuran (80 mL) at 0° C. was added 4-nitrophenyl chloroformate (5.83 g, 28.92 mmol) in one portion under nitrogen atmosphere. The reaction mixture was stirred at ambient temperature for 1.5 hours and filtered. The filter cake was washed with water (500 mL), tetrahydrofuran (300 mL) and hexanes (100 mL) and then dried to give the title compound as an off-white solid (6.169 g, 74%): ¹H NMR (300 MHz, DMSO-d₆) δ 11.33 (s, 1H), 8.55 (d, J=5.1 Hz, 1H), 8.37-8.28 (m, 3H), 7.61-7.54 (m, 3H), 3.89 (s, 3H); MS (ES−) m/z 317.9 (M+1).

### Preparation 1.1

### Preparation of ethyl 4-methyl-2-((4-nitrophenoxy)carbonylamino)thiazole-5-carboxylate

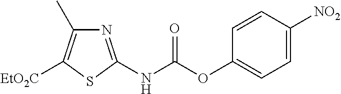

Following the procedure as described for methyl 2-((4-nitrophenoxy)carbonylamino)isonicotinate, making non-critical variations as required to replace methyl 2-aminopyridine-4-carboxylate with ethyl 2-amino-4-methylthiazole-5-carboxylate, the title compound was obtained as a beige in 78% yield ¹H NMR (300 MHz, DMSO-d₆) δ 8.33 (d, J=9.1 Hz, 2H), 7.59 (d, J=9.1 Hz, 2H), 4.24 (q, J=7.1 Hz, 2H), 2.55 (s, 3H), 1.26 (t, J=7.1 Hz, 3H).

### Preparation 2

### Preparation of tert-butyl 7-fluoro-4-oxospiro[chroman-2,4′-piperidine]-1′-carboxylate

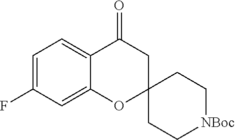

A mixture of 1-(4-fluoro-2-hydroxyphenyl)ethanone (6.140 g, 39.83 mmol), tert-butyl 4-oxopiperidine-1-carboxylate (7.94 g, 39.85 mmol) and pyrrolidine (3.30 mL 6.53 mmol) in methanol (80 ml) was stirred at ambient temperature for 16 hours under nitrogen atmosphere, then concentrated in vacuo. The residue was purified by column chromatography eluting with 10% ethyl acetate in hexanes, then with 30% ethyl acetate in hexanes to afford the title compound as a yellowish gum which solidified upon standing at ambient temperature for 1 day (7.252 g, 54%) ¹H NMR (300 MHz, CDCl₃) δ 7.88 (dd, J=8.7, 6.7 Hz, 1H), 6.77-6.64 (m, 2H), 3.87 (br s, 2H), 3.20 (t, J=12.1 Hz, 2H), 2.70 (s, 2H), 2.06-1.96 (m, 2H), 1.69-1.54 (m, 2H), 1.46 (s, 9H).

### Preparation 2.1

### Preparation of tert-butyl 8-chloro-4-oxospiro[chroman-2,4′-piperidine]-1′-carboxylate

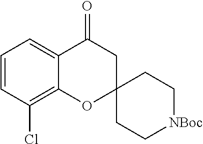

Following the procedure as described in PREPARATION 2, making non-critical variations as required to replace 1-(4-fluoro-2-hydroxyphenyl)ethanone with 1-(3-chloro-2-hydroxyphenyl)ethanone, the title compound was obtained as an off-white solid in 73% yield: ¹H NMR (300 MHz, CDCl₃) δ 7.77 (dd, J=7.8, 1.6 Hz, 1H), 7.57 (dd, J=7.8, 1.6 Hz, 1H), 7.00-6.92 (m, 1H), 3.95 (br s, 2H), 3.24 (t, J=11.6 Hz, 2H), 2.75 (s, 2H), 2.04 (d, J=12.9 Hz, 2H), 1.68-1.54 (m, 2H), 1.46 (s, 9H).

### Preparation 2.2

### Preparation of tert-butyl 6-fluoro-4-oxospiro[chroman-2,4′-piperidine]-1′-carboxylate

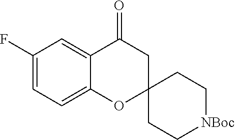

Following the procedure as described in PREPARATION 2, making non-critical variations as required to replace 1-(4-fluoro-2-hydroxyphenyl)ethanone with 1-(5-fluoro-2-hydroxyphenyl)ethanone, the title compound was obtained as an off-white solid in 56% yield: ¹H NMR (300 MHz, CDCl₃) δ 7.52 (dd, J=8.6, 3.7 Hz, 1H), 7.25-7.18 (m, 1H), 6.96 (dd, J=8.6, 3.7 Hz, 1H), 3.87 (br s, 2H), 3.19 (t, J=12.5 Hz, 2H), 2.71 (s, 2H), 2.01 (d, J=12.5 Hz, 2H), 1.68-1.53 (m, 2H), 1.46 (s, 9H).

### Preparation 3

### Preparation of 6-fluorospiro[chroman-2,4′-piperidine]

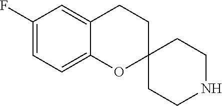

A. To a stirred solution of tert-butyl 6-fluoro-4-oxospiro[chroman-2,4′-piperidine]-1′-carboxylate (3.610 g, 10.76 mmol) in absolute ethanol (66 mL) was added sodium borohydride (2.849 g, 75.31 mmol) at ambient temperature under nitrogen atmosphere. The reaction mixture was stirred for 2 hours, carefully quenched with ice-cold saturated aqueous ammonium chloride solution (150 mL) and concentrated in vacuo to remove ethanol. The aqueous layer was extracted with ethyl acetate (150 mL). The organic layer was washed with water (2×150 mL), dried over anhydrous sodium sulfate, filtered and concentrated in vacuo to afford tert-butyl 6-fluoro-4-hydroxyspiro[chroman-2,4′-piperidine]-1′-carboxylate as a colorless foam (3.20 g, 88%) ¹H NMR (300 MHz, CDCl₃) δ 7.15 (dd, J=8.9, 2.6 Hz, 1H), 6.95-6.85 (m, 1H), 6.79 (dd, J=8.9, 4.7 Hz, 1H), 4.90-4.78 (m, 1H), 3.84 (br s, 2H), 3.36-3.06 (m, 2H), 2.14 (dd, 13.6, 6.2 Hz, 1H), 1.98-1.51 (m, 6H), 1.46 (s, 9H).

B. To a stirred mixture of tert-butyl 6-fluoro-4-hydroxyspiro[chroman-2,4′-piperidine]-1-carboxylate (3.146 g, 9.324 mmol) in trifluoroacetic acid (32 mL) at ambient temperature was added triethylsilane (14.9 mL, 93.28 mmol). The resulting reaction mixture was stirred for 16 hours, and then was concentrated in vacuo. The residue was stirred with diethyl ether/hexanes and the resulting precipitate was collected by filtration and washed with hexanes. The isolated solid was taken up in saturated aqueous sodium bicarbonate solution (150 mL) and extracted with dichloromethane (3×100 mL). The combined organic layer was dried over anhydrous sodium sulfate, filtered and concentrated in vacuo to afford the title compound as a yellowish semi-solid (1.89 g, 92%): ¹H NMR (300 MHz, CDCl₃) δ 6.84-6.72 (m, 3H), 3.38 (br s, 1H), 3.17-2.87 (m, 4H), 2.75 (t, J=6.8 Hz, 2H), 1.88-1.73 (m, 4H), 1.69-1.54 (m, 2H); MS (ES+) m/z 222.0 (M+1).

### Preparation 4

### Preparation of 7-fluorospiro[chroman-2,4′-piperidin]-4-one hydrochloride

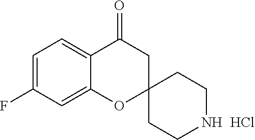

To a solution of tert-butyl 7-fluoro-4-oxospiro[chroman-2,4′-piperidine]-1′-carboxylate (3.000 g, 8.945 mmol) in 1,4-dioxane (18 mL) was added 4 M hydrochloric acid in 1,4-dioxane (9.0 mL, 36.0 mmol). The reaction mixture was stirred at reflux for 2 hours, cooled to ambient temperature and concentrated in vacuo. The residue was triturated with acetonitrile to afford the title compound as an off-white solid (2.22 g, 91%); ¹H NMR (300 MHz, DMSO-d) δ 9.09 (br s, 2H), 7.82 (dd, J=8.7, 6.8 Hz, 1H), 7.07 (dd, J=10.4, 2.3 Hz, 1H), 6.99-6.91 (m, 1H), 3.23-3.02 (m, 4H), 2.92 (s, 2H), 2.18-2.05 (m, 2H), 1.99-1.85 (m, 2H), MS (ES+) m/z 236.0 (M+1).

### Preparation 4.1

### Preparation of 8-chlorospiro[chroman-2,4′-piperidin]-4-one hydrochloride

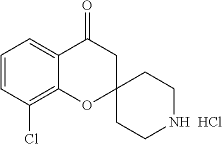

Following the procedure as described in PREPARATION 4, making non-critical variations as required to replace tert-butyl 7-fluoro-4-oxospiro[chroman-2,4′-piperidine]-1′-carboxylate with tert-butyl 8-chloro-4-oxospiro[chroman-2,4′-piperidine]-1′-carboxylate, the title compound was obtained as an off-white solid in 73% yield: ¹H NMR (300 MHz, DMSO-d₆) δ 9.17 (br s, 1H), 9.08 (br s, 1H), 7.80 (dd, J=7.8, 1.5 Hz, 1H), 7.73 (dd, J=7.8, 1.5 Hz, 1H), 7.15-7.08 (m, 1H), 3.31-3.18 (m, 2H), 3.12-2.93 (m, 4H), 2.14 (d, J=14.1 Hz, 2H), 2.03-1.88 (m, 2H); MS (ES+) m/z 252.0 (M+1), 254.0 (M+1).

### Preparation 5

### Preparation of tert-butyl 8-chloro-4-hydroxyspiro[chroman-2,4′-piperidine]-1′-carboxylate

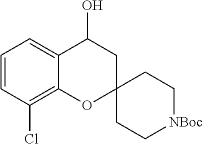

To a stirred solution of tert-butyl 8-chloro-4-oxospiro[chroman-2,4′-piperidine]-1′-carboxylate (2.000 g, 5.685 mmol) in absolute ethanol (50 mL) under nitrogen atmosphere was added sodium borohydride (1.505 g, 39.78 mmol). The reaction mixture was stirred for 2 hours, carefully quenched with saturated aqueous ammonium chloride solution (100 mL) and extracted with dichloromethane (2×150 mL). The combined organic layer was washed with water (150 mL), dried over anhydrous sodium sulfate, filtered and concentrated in vacuo to afford the title compound as a colorless foam (1.964 g, 98%)¹H NMR (300 MHz, CDCl₃) δ 7.34 (d, J=71, 1H), 7.27 (d, J=8.7 Hz, 1H), 6.92-6.85 (m, 1H), 4.93-4.84 (m, 1H), 3.90 (br s, 2H), 3.39-3.11 (m, 2H), 2.21-2.11 (m, 1H), 2.07-1.88 (m, 3H), 1.84-1.74 (m, 1H), 1.72-1.51 (m, 2H), 1.46 (s, 9H).

### Preparation 6

### Preparation of 8-chlorospiro[chroman-2,4′-piperidine]

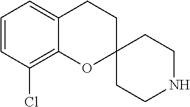

A mixture of tert-butyl 8-chloro-4-hydroxyspiro[chroman-2,4′-piperidine]-1′-carboxylate (1.909 g, 5.395 mmol) and trifluoroacetic acid (20 mL) was stirred at ambient temperature for 15 minutes, followed, by the addition of triethylsilane (8.62 mL, 53.97 mmol). The resulting reaction mixture was stirred at ambient temperature for 16 hours and concentrated. Saturated aqueous sodium bicarbonate solution (100 mL) was added to the residue. The aqueous layer was extracted with dichloromethane (2×100 mL), and the combined organic layer was dried over anhydrous sodium sulfate, filtered and concentrated in vacuo. The residue was purified by column chromatography eluting with dichloromethane/methanol/triethylamine (9:1:0.1) to afford the trifluoroacetic acid salt of the title compound. The salt obtained was dissolved in dichloromethane (50 mL), followed by the addition of 1 N sodium hydroxide solution (75 mL). The aqueous layer was extracted with dichloromethane (2×50 mL). The combined organic layer was dried over anhydrous sodium sulfate, filtered and concentrated in vacuo to afford the title compound as a yellow oil (0.935 g, 73%): ¹H NMR (300 MHz, CDCl₃) δ 7.16 (dd, J=7.8, 0.9 Hz, 1H), 6.93 (dd, J=7.8, 0.9 Hz, 1H), 6.77-6.70 (m, 1H), 3.19-3.07 (m, 2H), 2.94-2.84 (m, 2H), 2.79° (t, J=6.9 Hz, 2H), 2.27 (br s, 1H), 1.86-1.76 (m, 4H), 1.64-1.51 (m, 2H); MS (ES+) it 238.0 (M+1), 240.0 (M+1).

### Preparation 7

### Preparation of 7-fluorospiro[chromene-2,4′-piperidine]

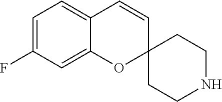

To a stirred solution of tert-butyl 7-fluoro-4-oxospiro[chroman-2,4′-piperidine]-1′-carboxylate (3.941 g, 11.75 mmol) in tetrahydrofuran (30 mL) at 0° C. under nitrogen atmosphere was added dropwise borane tetrahydrofuran complex (70.0 mL of 1.0 M in tetrahydrofuran, 70.0 mmol). The reaction mixture was stirred at reflux for 16 hours, then cooled to 0° C. and treated with 5 N aqueous hydrochloric acid solution (100 mL). The reaction mixture was stirred at reflux for 3 hours, allowed to cool to ambient temperature then concentrated in vacuo to remove tetrahydrofuran. The residual solution was cooled to 0° C. and quenched with 4 N aqueous sodium hydroxide solution. The aqueous layer was extracted with dichloromethane (3×100 mL), and the combined organic layer was dried over anhydrous sodium sulfate, filtered and concentrated in vacuo. The residue was purified by column chromatography, eluting with dichloromethane/methanol/triethylamine (9:1:0.1) to afford the title compound as an orange liquid (1.412 g, 55%): ¹H NMR (300 MHz, CDCl₃) δ 6.95-6.88 (m, 1H), 6.59-6.51 (m, 2H), 6.32 (d, J=9.8 Hz, 1H), 5.56 (d, J=9.8 Hz, 1H), 3.15-3.02 (m, 2H), 2.91-2.80 (m, 2H), 2.01-1.90 (m, 2H), 1.83-1.58 (m, 3H); MS (ES+) ink 220.1 (M+1).

### Preparation 8

### Preparation of methyl 2-(7-fluoro-4-oxospiro[chroman-2,4′-piperidine]-1′-ylcarboxamido)isonicotinate

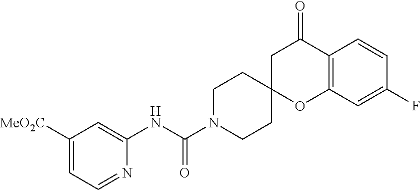

A mixture of 7-fluorospiro[chroman-2,4′-piperidin]-4-one hydrochloride (2.145 g, 7.894 mmol) and triethylamine (1.21 mL, 8.68 mmol) in N,N-dimethylformamide (25 mL) was stirred at ambient temperature for 15 minutes, followed by the addition of methyl 2-((4-nitrophenoxy)-carbonylamino)isonicotinate (2.505 g, 7.896 mmol). The reaction mixture was stirred at ambient temperature for 16 hours, diluted with ethyl acetate (75 mL) and washed with 1.85% aqueous hydrochloric acid solution (2×50 mL). The aqueous layer was extracted with ethyl acetate (2×50 mL), and the combined organic layer was washed with 1 N sodium hydroxide solution (2×50 mL), water (2×50 mL), dried over anhydrous sodium sulfate, filtered and concentrated in vacuo to yield the title compound as a yellow foam (2.324 g, 71%): ¹H NMR (300 MHz, CDCl₃) δ 8.54 (s, 1H), 8.31 (d, J=5.0 Hz, 1H), 7.95-7.85 (m, 1H), 7.51 (d, J=5.0 Hz, 1H), 7.43 (br s, 1H) 6.80-6.66 (m, 2H), 4.01-3.88 (m, 5H), 3.46-3.32 (m, 2H), 2.73 (s, 2H), 2.18-2.07 (m, 2H), 1.79-1.65 (m, 2H); MS (ES+) m/z 414.0 (M+1).

### Preparation 8.1

### Preparation of methyl 2-(8-chloro-4-oxospiro[chroman-2,4′-piperidine]-1′-ylcarboxamido)isonicotinate

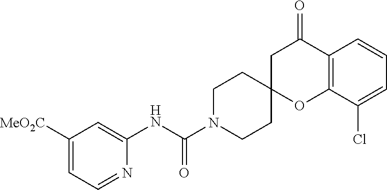

Following the procedure as described in PREPARATION 8, making non-critical variations as required to replace 7-fluorospiro[chroman-2,4′-piperidin]-4-one hydrochloride with 8-chlorospiro[chroman-2,4′-piperidin]-4-one hydrochloride, the title compound was obtained as an off-white foam in 84% yield: ¹H NMR (300 MHz, CDCl₃) δ 1.5 (s, 1H), 8.32 (d, J=5.1 Hz, 1H), 7.80 (dd, J=7.8, 1.5 Hz, 1H), 7.60 (dd, J=7.8, 1.5 Hz, 1H), 7.52 (dd, J=5.1, 1.3 Hz, 1H), 7.38 (br s, 1H), 7.03-6.96 (m, 1H), 4.01 (d, 13.3 Hz, 2H), 3.93 (s, 3H), 3.46 (t, J=12.3 Hz, 2H), 2.79 (s, 2H), 2.16 (d, J=13.3 Hz, 2H), 1.75 (dd, J=13.3, 4.5 Hz, 2H); MS (ES+) m/z 430.0 (M+1), 432.0 (M+1).

### Preparation 9

### Preparation of methyl 2-(7-fluorospiro[chromene-2,4′-piperidine]ylcarboxamido)isonicotinate

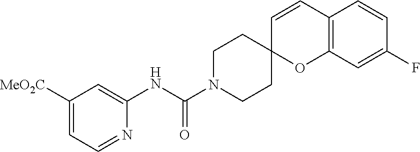

A mixture of 7-fluorospiro[chromene-2,4″-piperidine](1.412 cg, 6.44 mmol) and methyl 2-((4-nitrophenoxy)carbonylamino)isonicotinate (2.045 g, 6.446 mmol) in N,N-dimethylformamide (14 mL) was stirred at ambient temperature for 16 hours, diluted with ethyl acetate (75 mL) and washed with 1.85% aqueous hydrochloric acid solution (2×50 mL). The aqueous layer was extracted with ethyl acetate (2×50 mL), and the combined organic layer was washed with 1 N sodium hydroxide solution (2×50 mL), water (2×50 mL), dried over anhydrous sodium sulfate, filtered and concentrated in vacuo to give the title compound as a yellow foam (2.316 g, 90%): ¹H NMR (300 MHz, CDCl₃) δ 8.59-8.56 (m, 1H), 8.32 (dd, J=5.1, 0.7 Hz, 1H), 7.51 (dd, J=5.1, 1.4 Hz, 1H), 7.38 (br s, 1H), 6.95 (dd, 8.9, 6.4 Hz, 1H), 6.63-6.55 (m, 2H), 6.39 (d, J=9.8 Hz, 1H), 5.49 (d, 9.8 Hz, 1H), 3.97-3.87 (m, 5H), 3.53-3.40 (m, 2H), 2.15-2.04 (m, 2H), 1.77-1.64 (m, 2H); MS (ES+) m/z 398.0 (M+1).

### PREPARATION 9.1

### Preparation of methyl 2-(8-chlorospiro[chroman-2,4′-piperidine]-1′-ylcarboxamido)isonicotinate

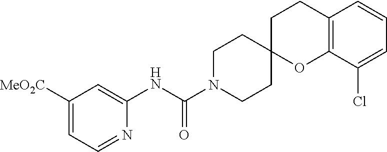

Following the procedure as described in PREPARATION 9, making non-critical variations as required to replace 7-fluorospiro[chromene-2,4′-piperidine] with 8-chlorospiro[chroman-2,4′-piperidine], the title compound was obtained as an off-white foam in 80% yield: ¹H NMR (300 MHz, CDCl₃) δ 8.59 (s, 1H), 8.32 (d, J=5.1 Hz, 1H), 7.51 (dd, J=5.1, 1.3 Hz, 1H), 7.39 (br s, 1H), 7.20 (d, J=7.7, 1H), 6.98 (d, J=7.7 Hz, 1H), 6.84-6.76 (m, 1H), 3.99 (d, J=13.0 Hz, 2H), 3.93 (s, 3H), 3.48 (t, J=13.0 Hz, 2H), 2.83 (t, J=6.9 Hz, 2H), 1.92 (d, J=13.0 Hz, 2H), 1.86 (t, J=6.9 Hz, 2H), 1.71-1.63 (m, 2H); MS (ES+) m/z 416.0 (M+1), 418.0 (M+1).

### Preparation 9.2

### Preparation of methyl 2-(6-fluorospiro[chroman-2,4′-piperidine]-1′-ylcarboxamido)isonicotinate

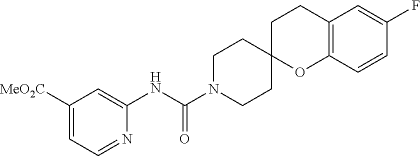

Following the procedure as described in PREPARATION 9, making non-critical variations as required to replace 7-fluorospiro[chromene-2,4′-piperidine] with 6-fluorospiro[chroman-2,4′-piperidine], the title compound was obtained as a yellow foam in 73%: ¹H NMR (300 MHz, CDCl₃) δ 8.58 (s, 1H), 8.32 (d, J=5.1 Hz, 1H), 7.50 (dd, J=5.1, 1.4 Hz, 1H), 7.38 (br s, 1H), 6.87-6.74 (m, 3H), 3.99-3.88 (m, 5H), 3.47-3.32 (m, 2H), 2.78 (t, J=6.8 Hz, 2H), 1.89 (d, J=12.9 Hz, 2H), 1.81 (t, J=6.8 Hz, 2H), 1.68-1.56 (m, 2H); MS (ES+) m/z 400.0 (M+1).

### Preparation 9.3

### Preparation of methyl 6-(7-fluorospiro[chroman-2,4′-piperidine]-1′-ylcarboxamido)picolinate

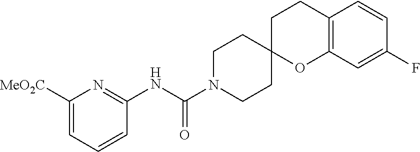

Following the procedure as described in PREPARATION 9, making non-critical variations as required to replace methyl 2-((4-nitrophenoxy)carbonylamino)isonicotinate with methyl 6-((4-nitrophenoxy)carbonylamino)picolinate and to replace 7-fluorospiro[chromene-2,4′-piperidine] with 7-fluorospiro[chroman-2,4′-piperidine]hydrochloride, the title compound was obtained as a colorless foam in 65% yield: ¹H NMR (300 MHz, CDCl₃) δ 8.27 (dd, J=7.0, 2.3 Hz, 1H), 7.82-7.74 (m, 2H), 7.47 (br s, 1H), 7.05-6.97 (m, 1H), 6.63-6.54 (m, 2H), 4.02-3.91 (m, 5H), 3.46-3.32 (m, 2H), 2.76 (t, J=6.8 Hz, 2H), 1.89 (d, J=13.0 Hz, 2H), 1.82 (t, J=6.8 Hz, 2H), 1.67-1.54 (m, 2H); MS (ES+) m/z 400.1 (M+1).

### Preparation 9.4

### Preparation of methyl 5-(7-fluorospiro[chroman-2,4′-piperidine]-1′-ylcarboxamido)nicotinate

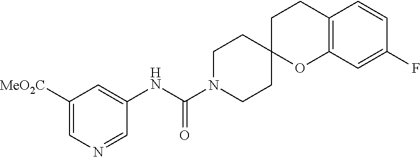

Following the procedure as described in PREPARATION 9, making non-critical variations as required to replace methyl 2-((4-nitrophenoxy)carbonylamino)isonicotinate with methyl 5-((4-nitrophenoxy)carbonylamino)nicotinate and to replace 7-fluorospiro[chromene-2,4′-piperidine] with 7-fluorospiro[chroman-2,4′-piperidine]hydrochloride, the title compound was obtained as a yellow foam in 76% yield: ¹H NMR (300 MHz, CDCl₃) δ 8.87 (d, J=1.0 Hz, 1H), 8.72 (d, J=2.4 Hz, 1H), 8.46-8.43 (m, 1H), 7.05-6.97 (m, 1H), 6.73 (br s, 1H), 6.64-6.55 (m, 2H), 3.97-3.87 (m, 5H), 3.46-3.34 (m, 2H), 2.76 (t, J=6.7 Hz, 2H), 1.90 (d, J=12.9 Hz, 2H), 1.83 (t, J=6.7 Hz, 2H), 1.71-1.58 (m, 2H); MS (ES+) m/z 400.0 (M+1).

### Preparation 9.5

### Preparation of methyl 2-(1,3-dihydrospiro[indene-2,4′-piperidine]-1′-ylcarboxamido)isonicotinate

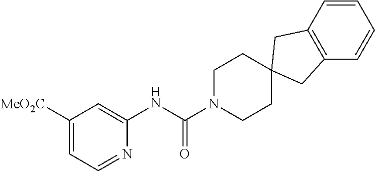

Following the procedure as described in PREPARATION 9, making non-critical variations as required to replace 7-fluorospiro[chromene-2,4′-piperidine] with 1,3-dihydrospiro[indene-2,4′-piperidine], the title compound was obtained as a colorless foam in 56%; ¹H NMR (300 MHz, CDCl₃) δ 8.59 (s, 1H), 8.32 (d, J=5.1 Hz, 1H), 7.51 (dd, J=5.1, 1.3 Hz, 1H), 7.35 (br s, 1H), 7.22-7.12 (m, 4H), 3.93 (s, 3H), 3.62-3.54 (m, 4H), 2.87 (s, 4H), 1.74-1.66 (m, 4H); MS (ES+) m/z 366.0 (M+1).

### Preparation 9.6

### Preparation of ethyl 2-(7-fluorospiro[chroman-2,4′-piperidine]-1′-ylcarboxamido)-4-methylthiazole-5-carboxylate

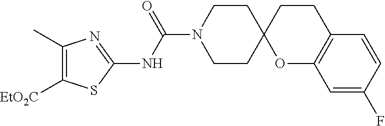

Following the procedure as described in PREPARATION 9, making non-critical variations as required to replace methyl 2-((4-nitrophenoxy)carbonylamino)isonicotinate with 4-methyl-2-((4-nitrophenoxy)carbonylamino)thiazole-5-carboxylate and to replace 7-fluorospiro[chromene-2,4′-piperidine] with 7-fluorospiro[chroman-2,4′-piperidine]hydrochloride, the title compound was obtained as a colorless foam in 85% yield: ¹H NMR (300 MHz, CDCl₃) δ 8.66 (br s, 1H), 7.04-6.96 (m, 1H), 6.63-6.52 (m, 2H), 4.29 (q, J=7.1 Hz, 2H), 3.90 (d, J=12.7 Hz, 2H), 3.40 (t, J=12.7 Hz, 2H), 2.75 (t, J=6.7 Hz, 2H), 2.59 (s, 3H), 1.90 (d, J=13.2 Hz, 2H), 1.81 (t, J=6.7 Hz, 2H), 1.67-1.55 (m, 2H), 1.33 (t, J=7.1 Hz, 3H); MS (ES+) m/z 434.0 (M+1).

### Preparation 10

### Preparation of methyl 2-(7-fluorospiro[chroman-2,4′-piperidine]-1′-ylcarboxamido)isonicotinate

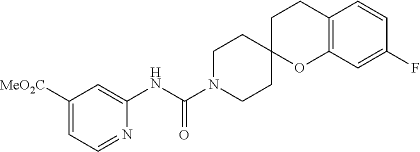

A mixture of methyl 2-(7-fluorospiro[chromene-2,4′-piperidine]-1′-ylcarboxamido)-isonicotinate (2.316 g, 5.828 mmol) and 10% palladium on carbon (0.232 g) in ethanol (20 mL) and tetrahydrofuran (20 mL) was shaken on a Parr hydrogenator at 55 psi for 18 hours. The reaction mixture was filtered through a pad of Celite. The pad was washed with ethyl acetate, and the filtrate was concentrated. The residue was purified by column chromatography eluting with 50% ethyl acetate in hexanes to afford the title compound as a yellowish foam (2.110 g, 91%); ¹H NMR (300 MHz, CDCl₃) δ 8.58 (s, 1H), 8.31 (dd, J=5.1, 0.6 Hz, 1H), 7.50 (dd, 5.1, 1.4 Hz, 1H), 7.41 (br s, 1H), 7.05-6.95 (m, 1H), 6.62-6.54 (m, 2H), 4.00-3.90 (m, 5H), 3.46-3.34 (m, 2H), 2.75 (t, J=6.8 Hz, 2H), 1.94-1.86 (m, 2H), 1.82 (t, J=6.8 Hz, 2H), 1.70-1.57 (m, 2H), MS (ES+) m/z 400.1 (M+1).

### Preparation 11

### Preparation of 2-(7-fluoro-4-oxospiro[chroman-2,4′-piperidine]-1′-ylcarboxamido)isonicotinic acid

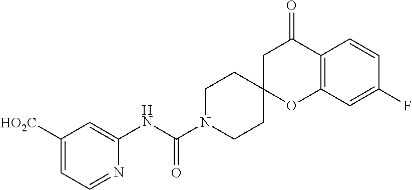

A mixture of methyl 2-(7-fluoro-4-oxospiro[chroman-2,4′-piperidine]-1′-yl-carboxamido)isonicotinate (1.888 g, 4.567 mmol) and lithium hydroxide monohydrate (0.287 g, 6.840 mmol) in tetrahydrofuran (12 mL) and water (3 mL) was stirred at ambient temperature for 30 minutes, then diluted with water, acidified with 10% aqueous acetic acid solution to pH ˜6 and concentrated to remove tetrahydrofuran. The resulting precipitate was collected by filtration, washed with water and dried in vacuo to afford the title compound as an off-white solid (1.247 g, 68%): ¹H NMR (300 MHz, DMSO-d₆) δ 13.53 (br s, 1H), 9.54 (s, 1H), 8.38 J=5.1 Hz, 1H), 8.28 (s, 1H), 7.81 (dd, J=8.6, 6.9 Hz, 1H), 7.38 (dd, J=5.1, 0.9 Hz, 1H), 7.02 (dd, J=10.3, 2.4 Hz, 1H), 6.97-6.88 (m, 1H), 4.02-3.89 (m, 2H), 3.31-3.19 (m, 2H), 2.87 (s, 2H), 1.98-1.87 (m, 2H), 1.79-1.65 (m, 2H); MS (ES+) m/z 400.0 (M+1).

### Preparation 11.1

### Preparation of 2-(7-fluorospiro[chroman-2,4′-piperidine]-1′-ylcarboxamido)isonicotinic acid

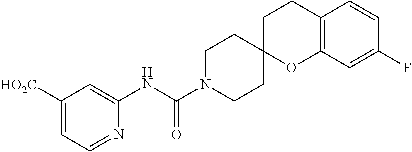

Following the procedure as described in PREPARATION 11, making non-critical variations as required to replace methyl 2-(7-fluoro-4-oxospiro[chroman-2,4′-piperidine]-1′-ylcarboxamido)isonicotinate with methyl 2-(7-fluorospiro[chroman-2,4′-piperidine]-1′-ylcarboxamido)isonicotinate, the title compound was obtained as a colorless solid in 91% yield: ¹H NMR (300 MHz, DMSO-d₆) δ 13.52 (br s, 1H), 9.50 (s, 1H), 8.38 (d, J=5.0, Hz, 1H), 8.29 (s, 1H), 7.38 (d, J=50 Hz, 1H), 7.16-7.06 (m, 1H), 6.72-6.61 (m, 2H), 3.93 (d, J=13.4 Hz, 2H), 3.32-3.18 (m, 2H), 2.71 (t, J=6.3 Hz, 2H), 1.81 (t, J=63 Hz, 2H), 1.76-1.55 (m, 4H); MS (ES+) m/z 386.0 (M+1).

### Preparation 11.2

### Preparation of 2-(8-chlorospiro[chroman-2,4′-piperidine]-1′-ylcarboxamido)isonicotinic acid

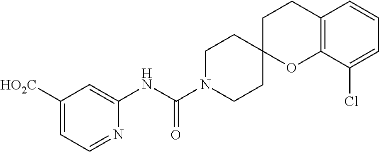

Following the procedure as described in PREPARATION 11, making non-critical variations as required to replace methyl 2-(7-fluoro-4-oxospiro[chroman-2,4′-piperidine]-1′-ylcarboxamido)isonicotinate with methyl 2-(8-chlorospiro[chroman-2,4′-piperidine]-1′-ylcarboxamido)isonicotinate, the title compound was obtained as an off-white solid in 94% yield: ¹H NMR (300 MHz, DMSO-d₆) δ 9.44 (s, 1H), 8.34 (d, J=5.1 Hz, 1H), 8.26 (s, 1H), 7.36 (dd, J=5.1, Hz, 1H), 7.23 (d, J=7.8, 1H), 7.07 (d, J=7.8 Hz, 1H), 6.87-6.79 (m, 1H), 4.03 (d, J=13.4 Hz, 2H), 3.21 (t, J=13.4 Hz, 2H), 2.79 (t J=6.6 Hz, 2H), 1.84 (t, J=6.6 Hz, 2H), 1.79-1.57 (m, 4H); MS (ES+) m/z 402.1 (M+404.0 (M+1).

### Preparation 11.3

### Preparation of 2-(8-chloro-4-oxospiro[chroman-2,4′-piperidine]-1′ylcarboxamido)isonicotinic acid

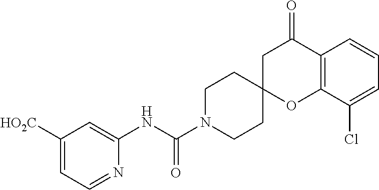

Following the procedure as described in PREPARATION 11, making non-critical variations as required to replace methyl 2-(7-fluoro-4-oxospiro[chroman-2,4′-piperidine]-1′-ylcarboxamido)isonicotinate with methyl 2-(8-chloro-4-oxospiro[chroman-2,4′-piperidine]-1′-ylcarboxamido)isonicotinate, the title compound was obtained as a yellowish solid in 86% yield: ¹H NMR (300 MHz, DMSO-d₆) δ 9.54 (s, 1H), 8.37 (d, J=4.9 Hz, 1H), 8.28 (s, 1H), 7.78 (d, J=7.8, 1H), 7.72 (d 7.8 Hz, 1H), 7.38 (d, J=49 Hz, 1H), 7.13-7.05 (m, 1H), 4.05 (d, J=13.6 Hz, 2H), 3.19 (t, J=12.1 Hz, 2H), 2.95 (s, 2H), 1.95 (d, J=13.6 Hz, 2H), 1.88-1.68 (m, 2H); MS (ES+) m/z 416.0 (M+1), 418.0 (M+1).

### Preparation 11.4

### Preparation of 2-(6-fluorospiro[chroman-2,4′-piperidine]-1′-ylcarboxamido)isonicotinic acid

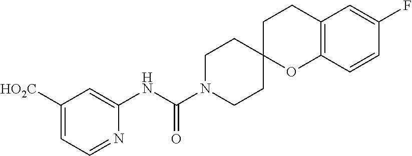

Following the procedure as described in PREPARATION 11, making non-critical variations as required to replace methyl 2-(7-fluoro-4-oxospiro[chroman-2,4′-piperidine]-1′-ylcarboxamido)isonicotinate with methyl 2-(6-fluorospiro[chroman-2,4′-piperidine]-1′-ylcarboxamido)isonicotinate, the title compound was obtained as an off-white solid in 97% yield: ¹H NMR (300 MHz, DMSO-d₆) δ 9.36 (s, 1H), 8.30 (d, J=4.7 Hz, 1H), 8.23 (s, 1H), 7.36 (d, J=4.7 Hz, 1H), 7.00-6.76 (m, 3H), 3.92 (d, J=12.7 Hz, 2H), 3.23 (t, J=110.3 Hz, 2H), 2.79-2.68 (m, 2H), 1.85-1.51 (m, 6H); MS (ES+) m/z 386.0 (M+1).

### Preparation 11.5

### Preparation of 6-(7-fluorospiro[chroman-2,4′-piperidine]-1′-ylcarboxamido)picolinic acid

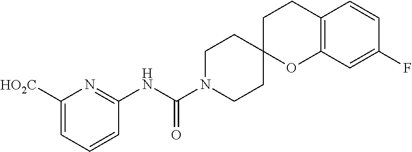

Following the procedure as described in PREPARATION 11, making non-critical variations as required to replace methyl 2-(7-fluoro-4-oxospiro[chroman-2,4′-piperidine]-1′-ylcarboxamido)isonicotinate with methyl 6-(7-fluorospiro[chroman-2,4′-piperidine]-1′-ylcarboxamido)picolinate, the title compound was obtained as a colorless solid in 95% yield: MS (ES+) m/z 386.0 (M+1).

### Preparation 11.6

### Preparation of 5-(7-fluorospiro[chroman-2,4′-piperidine]-1′-ylcarboxamido)nicotinic acid

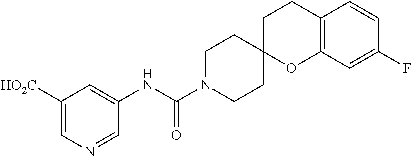

Following the procedure as described in PREPARATION 11, making non-critical variations as required to replace methyl 2-(7-fluoro-4-oxospiro[chroman-2,4′-piperidine]-1′-ylcarboxamido)isonicotinate with methyl 5-(7-fluorospiro[chroman-2,4′-piperidine]-1′-ylcarboxamido)nicotinate, the title compound was obtained as an off-white solid in 86% yield: ¹H NMR (300 MHz, DMSO-d₆) δ 8.96 (s, 1H), 8.87 (d, J=2.1 Hz, 1H), 8.63 (s, 1H), 8.42 (s, 1H), 7.16-7.07 (m, 1H), 6.71-6.62 (m, 2H), 3.91 (d, J=13.6 Hz, 2H), 3.41-3.20 (m, 2H), 2.72 (t, J=6.3 Hz, 2H), 1.82 (t, J=6.3 Hz, 2H), 1.74 (d, J=13.6 Hz, 2H), 1.69-1.57 (m, 2H); MS (ES+) m/z 386.0 (M+1).

### Preparation 11.7

### Preparation of 2-(1,3-dihydrospiro[indene-2,4′-piperidine]-1′-ylcarboxamido)isonicotinic acid

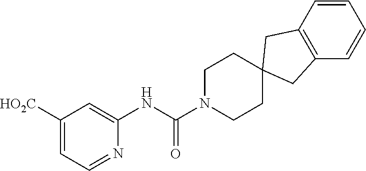

Following the procedure as described in PREPARATION 11, making non-critical variations as required to replace methyl 2-(7-fluoro-4-oxospiro[chroman-2,4′-piperidine]-1′-ylcarboxamido)isonicotinate with methyl 2-(1,3-dihydrospiro[indene-2,4′-piperidine]-1′-ylcarboxamido)isonicotinate, the title compound was obtained as a colorless solid in 94% yield: MS (ES+) m/z 352.0 (M+1).

### Preparation 12

### Preparation of 7-fluorospiro[chroman-2,4′-piperidine]hydrochloride

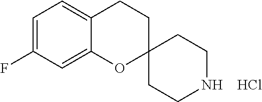

A Following the procedure as described PREPARATION 5, making non-critical variations as required to replace tert-butyl 8-chloro-4-oxospiro[chroman-2,4′-piperidine]-1′-carboxylate with tert-butyl 7-fluoro-4-oxospiro[chroman-2,4′-piperidine]-1′-carboxylate, tert-butyl 7-fluoro-4-hydroxyspiro[chroman-2,4′-piperidine]1′-carboxylate was obtained as a colorless solid in 95% yield: ¹H NMR (300 MHz, CDCl₃) δ 7.42-7.35 (m, 1H), 6.71-6.62 (m, 1H), 6.56 (dd, J=10.2, 2.4 Hz, 1H), 4.89-4.79 (m, 1H), 3.85 (br 2H), 3.34-3.07 (m, 2H), 2.13 (dd, J=13.7, 6.0 Hz, 1H), 2.00-1.51 (m, 6H), 1.46 (s, 9H).

B. A mixture of tert-butyl 7-fluoro-4-hydroxyspiro[chroman-2,4′-piperidine]-1′-carboxylate (15.60 g, 46.24 mmol) and trifluoroacetic acid (74 mL) was stirred at ambient temperature for 15 minutes. Triethylsilane (74.0 mL, 463.3 mmol) was added, and the resulting reaction mixture was stirred for 16 hours. The mixture was concentrated, and to the residue was added 1 N aqueous potassium hydroxide solution (400 mL). The aqueous layer was extracted with dichloromethane (2×300 mL), and the combined organic layer was dried over anhydrous sodium sulfate, filtered and concentrated in vacuo. The residue was purified by column chromatography eluting with dichloromethane/methanol/triethylamine (95:5:0.5) and further triturated with hexanes to afford a mixture of trifluoroacetic acid salt and free base of 7-fluorospiro[chroman-2,4′-piperidine] along with trifluoroacetic acid salt and free base of 7-fluorospiro[chromene-2,4′-piperidine]. The mixture of four compounds was stirred with 4 M hydrochloric acid in dioxane (50 mL) at ambient temperature for 1 hour, and then was concentrated. The residue was triturated with ether and the solid was collected by filtration to afford a beige solid. The solid was taken up in 1 N aqueous potassium hydroxide solution (300 mL) and extracted with dichloromethane (2×150 mL). The combined organic layer was dried over anhydrous sodium sulfate, filtered and concentrated in vacuo to give ca. 7 g of oil, which solidified upon standing at ambient temperature. The above material was dissolved in ethanol (30 mL) and tetrahydrofuran (30 mL) and was hydrogenated in a Parr apparatus at 55 psi with 10% palladium on carbon (0.700 g, 10% by weight) for 24 hours. The reaction mixture was filtered through a pad of Celite, the pad was washed with dichloromethane and the filtrate was concentrated. The residue was stirred with 4 M hydrochloric acid in dioxane (25 mL) for 30 minutes, and then concentrated. The residue was dissolved in methanol and precipitated with ether to afford the title compound as an off-white solid (5.553 g, 46%): ¹H NMR (300 MHz, CDCl₃) δ 9.74 (br s, 1H), 9.58 (br s, 1H), 7.05-6.97 (m, 1H), 6.66-6.52 (m, 2H), 3.45-3.25 (m, 4H), 2.75 (t, J=6.5 Hz, 2H), 2.17-1.84 (m, 6H); MS (ES+) m/z 222.0 (M+1).

### Preparation 13

### Preparation of methyl 6-((4-nitrophenoxy)carbonylamino)picolinate

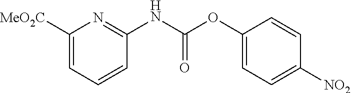

A. A mixture of 6-aminopicolinic acid (2.000 g, 14.48 mmol) and concentrated sulfuric acid (2 mL) in anhydrous methanol (20 mL) was stirred at reflux for 24 hours. The reaction mixture was allowed to cool to ambient temperature and was quenched with saturated aqueous sodium bicarbonate solution (150 mL). The aqueous layer was extracted with dichloromethane (2×100 mL). The combined organic layer was dried over anhydrous sodium sulfate, filtered and concentrated in vacuo to afford methyl 6-aminopicolinate as a colorless solid (1.563 g, 71%): ¹H NMR (300 MHz, CDCl₁) δ 7.58-7.47 (m, 2H), 6.67 (d, J=7.90 Hz, 1H), 4.76 (br s, 2H), 3.95 (s, 3H).

B. To a stirred solution of methyl 6-aminopicolinate (0.955 g, 6.277 mmol) in dichloromethane (50 mL) at 0° C. under nitrogen atmosphere was added pyridine (0.770 mL, 9.520 mmol), followed by the addition of by 4-nitrophenyl chloroformate (1.392 g, 6.906 mmol). The reaction mixture was stirred at 0° C. for 1 hour and then at ambient temperature for another hour, then filtered. The collected solid was washed with water, dichloromethane and dried. The solid was stirred in dichloromethane (40 mL) for 30 minutes, collected by filtration and dried to afford the title compound as a colorless solid (1.911 g, 96%): ¹H NMR (300 MHz, DMSO-d₆) δ 11.40 (s, 1H), 8.35-8.28 (m, 2H), 8.11-7.99 (m, 2H), 7.82 (dd, J=6.9, 1.4 Hz, 1H), 7.57-7.50 (m, 2H), 3.89 (s, 3H); MS (ES+) m/z 340.0 (M+23).

### Preparation 14

### Preparation of methyl 5-((4-nitrophenoxy)carbonylamino)nicotinate

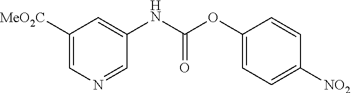

A. Following the procedure as described in Step A of PREPARATION 12, making non-critical variations as required to replace 6-aminopicolinic acid with 5-aminonicotinic acid, methyl 5-aminonicotinate was obtained as a colorless solid in 80% yield: δ ¹H NMR (300 MHz, DMSO-d₆) δ 8.24 (d, J=1.7 Hz, 1H), 8.12 (d, J=2.7 Hz, H), 7.44-7.41 (m, 1H), 5.66 (br s, 2H), 3.83 (5, 3H).

B. To a stirred solution of methyl 5-aminonicotinate (3.503 g, 23.02 mmol) in dichloromethane (350 mL) at ambient temperature was added pyridine (2.80 mL, 34.62 mmol). The resulting mixture was cooled to 0° C., followed by the addition of 4-nitrophenyl chloroformate (5.104 g, 25.32 mmol) under nitrogen atmosphere. The cooling bath was removed, and the reaction mixture was stirred for 2 hours and then filtered. The solid was washed with water, ether and dried to afford the title compound as a colorless solid (6.376 g, 87%): ¹H NMR (300 MHz, DMSO-d₆) δ 10.97 (s, 1H), 8.89 (d, J=2.5 Hz, 1H), 8.80 (d, J=1.7 Hz, 1H), 8.51 (s, 1H), 8.33 (d, J=9.1 Hz, 2H), 7.59 (d, J=9.1 Hz, 2H), 3.89 (s, 3H).

### Preparation 16

### Preparation of 2,2′-(benzylazanediyl)diethanol

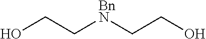

A mixture of diethanol amine (12.10 g, 115.1 mmol), benzyl bromide (13.70 mL, 115.3 mmol) and potassium carbonate (31.81 g, 230.2 mmol) in acetone (120 mL) was stirred at reflux for 18 hours, and then was allowed to cool to ambient temperature. The solid was removed by filtration, and the filtrate was concentrated in vacuo. The residue was purified by column chromatography, eluted with 10% methanol in dichloromethane to give the title compound as a yellowish oil (16.39 g, 73%): ¹H NMR (300 MHz, CDCl₃) δ 7.36-7.23 (m, 5H), 3.70 (s, 2H), 3.62 (t J=5.3 Hz, 4H), 2.72 (t, J=5.3 Hz, 4H), 2.48 (br s, 2H).

### Preparation 16

### Preparation of 1,3-dihydrospiro[indene-2,4′-piperidine]

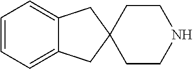

A. To a stirred solution of 2,2′-(benzylazanediyl)diethanol (14.60 g, 7477 mmol) in toluene (140 mL at 0° C. was slowly added phosphorus tribromide (21.1 mL, 224.5 mmol) under nitrogen atmosphere. The resulting paste was stirred at reflux for 6 hours, and then was allowed to cool to ambient temperature. The reaction mixture was quenched with ice-cold water (400 mL) and filtered. The organic phase of the filtrate was separated. The filter cake was washed with 4 N aqueous sodium hydroxide solution (400 and the filtrate was extracted with dichloromethane (3×100 mL). The combined organic layer was dried over anhydrous sodium sulfate, filtered and concentrated in vacuo. The residue was purified by column chromatography and eluted with 5% ethyl acetate in hexanes to afford N-benzyl-2-bromo-N-(2-bromoethyl)ethanamine as a colorless liquid (13.30 g, 55%): ¹H NMR (300 MHz, CDCl₃) δ 7.36-7.25 (m, 5H), 3.73 (s, 2H), 3.34 (t, J=7.3 Hz, 4H), 2.98 (t, 7.3 Hz, 4H).

B. 1-Indanone (1.895 g, 14.34 mmol) and freshly purified N-benzyl-2-bromo-N-(2-bromoethyl)ethanamine (6.905 g, 21.51 mmol) were combined in N,N-dimethylformamide at ambient temperature and heated to 50° C. under nitrogen atmosphere. Sodium hydride (60% in mineral oil, 1.434 g, 35.85 mmol) was added, and the resulting reaction mixture was stirred at 50° C. for 18 hours, cooled to ambient temperature, quenched with water (75 mL) and extracted with ethyl acetate (150 mL). The organic layer was washed with water, dried over anhydrous sodium sulfate, filtered and concentrated in vacuo. The residue was purified by column chromatography eluting with 50% ethyl acetate in hexanes to afford 1′-benzylspiro[indene-2,4′-piperidin]-1(3H)-one as a cream solid (0.752 g, 18%): ¹H NMR (300 MHz, CDCl₃) δ 7.76 (d, J=7.6 Hz, 1H), 7.63-7.56 (m, 1H), 7.45 (d, J=7.6 Hz, 1H), 7.41-7.22 (m, 6H), 3.56 (s, 2H), 3.03 (s, 2H), 2.98-2.89 (m, 2H), 2.24-2.00 (m, 4H), 1.37 (d, J=1.8 Hz, 2H); MS (ES+) m/z 292.1 (M+1).

C. To a stirred solution of 1′-benzylspiro[indene-2,4′-piperidin]-1(3H)-one (0.752 g, 2.581 mmol) in absolute ethanol (28 mL) at ambient temperature under nitrogen atmosphere was added sodium borohydride (0.683 g, 18.05 mmol). The reaction mixture was stirred for 2 hours, cooled to 0° C. and carefully quenched with saturated aqueous ammonium chloride solution (75 mL). The mixture was concentrated in vacuo to remove ethanol, and the aqueous layer was extracted with ethyl acetate (2×50 mL), and then with dichloromethane (5×50 mL). The combined dichloromethane layer was dried over anhydrous sodium sulfate, filtered and concentrated in vacuo to afford t-benzyl-1,3-dihydrospiro[indene-2,4′-piperidin]-1-ol as a colorless foam (0.700 g, 92%); MS (ES+) m/z 294.2 (M+1).

D. To a stirred solution of 1′-benzyl-1,3-dihydrospiro[indene-2,4′-piperidin]-1-ol (1.400 g, 4.772 mmol) in trifluoroacetic acid (15 mL) at ambient temperature was added triethylsilane (8.0 mL, 50.08 mmol). The resulting reaction mixture was stirred for 16 hours, and then concentrated in vacuo. The residue was taken up in 1 N aqueous sodium hydroxide solution (50 mL) and extracted with dichloromethane (2×75 mL). The combined organic layer was concentrated in vacuo, and the residue was dissolved in ethyl acetate (75 mL) and washed with brine (2×50 mL). The organic layer was dried over anhydrous sodium sulfate, filtered and concentrated in vacuo to afford 1′-benzyl-1,3-dihydrospiro[indene-2,4′-piperidine] as a colorless solid (0.947 g, 72%): ¹H NMR (300 MHz, CDCl₃) δ 7.36-7.23 (m, 5H), 7.19-7.10 (m, 4H), 3.54 (s, 2H), 2.80 (s, 4H), 2.46 (br s, 4H), 1.66 (t, J=5.5 Hz, 4H), MS (ES+) m/z 278.2 (M+1).

E. A mixture of 1′-benzyl-1,3-dihydrospiro[indene-2,4′-piperidine](0.947 g, 3.414 mmol) and ethyl chloroformate (0.430 mL) in toluene (10 mL) was stirred at reflux for 18 hours under nitrogen atmosphere. The mixture was allowed to cool to ambient temperature, diluted with ethyl acetate (75 mL), washed with saturated aqueous sodium bicarbonate solution (50 mL) and brine (50 mL). The organic layer was dried over anhydrous sodium sulfate, filtered and concentrated in vacuo to afford a light yellow oil which was stirred in a mixture of ethanol (10 and 50% aqueous potassium hydroxide solution (5 mL) at reflux for 20 hours. The reaction mixture was allowed to cool to ambient temperature and concentrated to remove ethanol. The residue was diluted with water (35 mL) and extracted with dichloromethane (2×50 mL). The combined organic layer was dried over anhydrous sodium sulfate, filtered and concentrated in vacuo. The residue was purified by column chromatography eluting with dichloromethane/methanol/triethylamine (9/1/0.1) to afford the title compound as a yellowish semisolid (0.492 g, 77%) ¹H NMR (300 MHz, CDCl₃) δ 7.21-7.09 (m, 4H), 2.91-2.85 (m, 4H), 2.81 (s, 4H), 1.72 (br s, 1H), 1.63-1.55 (m, 4H); MS (ES+) m/z 188.2 (M+1).

### Preparation 17

### Preparation of 2-(7-fluorospiro[chroman-2,4′-piperidine]-1′-ylcarboxamido)-4-methylthiazole-5-carboxylic acid

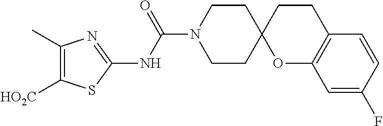

A mixture of ethyl 2-(7-fluorospiro[chroman-2,4′-piperidine]-1′-ylcarboxamido)-4-methylthiazole-5-carboxylate (1.176 g, 2.713 mmol) and 1 N aqueous sodium hydroxide solution (5.4 mL, 5.4 mmol) in ethanol (11 mL) was stirred at reflux for 2 hours. Additional 1 N sodium hydroxide solution was added (2.2 mL), and the reaction mixture was stirred at reflux for another 3 hours. The reaction mixture was allowed to cool to ambient temperature and diluted with water (75 mL). The aqueous layer was extracted with ethyl acetate (75 mL) and then acidified with 10% aqueous hydrochloric acid. The resulting precipitate was collected by filtration, washed with water and ether and dried to afford the title compound as an off-white solid (0.446 g, 40%): ¹H NMR (300 MHz, DMSO-d₆) δ 7.16-7.06 (m, 1H), 6.72-6.60 (m, 2H), 3.99 (d, J=13.3 Hz, 2H), 3.29-3.19 (m, 2H), 2.70 (t, J=6.0 Hz, 2H), 2.47 (s, 3H), 1.85-1.51 (m, 6H); (ES+) m/z 406.0 (M+1).

### Example 1

### Synthesis of 7-fluoro-N-(4-(methylcarbamoyl)pyridin-2-yl)-4-oxospiro[chroman-2,4′-piperidine]-1′-carboxamide hydrochloride

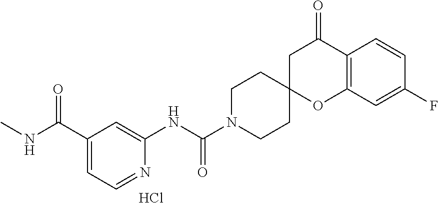

A. To a mixture of 2-(7-fluoro-4-oxospiro[chroman-2,4′-piperidine]-1′-ylcarboxamido)isonicotinic acid (1.395 g, 3.493 mmol), methylamine hydrochloride (1.179 g, 17.46 mmol), 1-hydroxybenzotriazole (0.708 g, 5.239 mmol) and 1-ethyl-3-(3-dimethylaminopropyl)carbodiimide hydrochloride (1.004 g, 5.237 mmol) in N,N-dimethylformamide (25 mL) was added N,N-diisopropylethylamine (5.00 mL, 28.70 mmol). The reaction mixture was stirred for 16 hours at ambient temperature, diluted with ethyl acetate (75 ml) and washed with saturated aqueous sodium bicarbonate solution (50 mL). The aqueous layer was extracted with ethyl acetate (50 mL), and the combined organic layer was washed with water (50 mL), dried over anhydrous sodium sulfate, filtered and concentrated in vacuo. The residue was purified by column chromatography eluting with 4% methanol in dichloromethane to afford 7-fluoro-N-(4-(methylcarbamoyl)pyridin-2-yl)-4-oxospiro[chroman-2,4′-piperidine]-1-carboxamide as a yellowish foam (0.844 g, 58%); ¹H NMR (300 MHz, CDCl₃) δ 8.30 (d, J=5.1 Hz, 1H), 8.23 (s, 1H), 7.91 (dd, J=8.7, 6.6 Hz, 1H), 7.45-7.39 (m, 2H), 6.81-6.67 (m, 2H), 6.48 (br s, 1H), 3.94 (d, J=13.4 Hz, 2H), 3.47-3.33 (m, 2H), 2.98 (d, J=4.9 Hz, 3H), 2.74 (s, 2H), 2.14 (d, J=13.4 Hz, 2H), 1.78-1.65 (m, 2H); MS (ES+) m/z 413.1 (M+1).

B. A mixture of 7-fluoro-N-(4-(methylcarbamoyl)pyridin-2-yl)-4-oxospiro[chroman-2,4′-piperidine]-1′-carboxamide (0.200 g, 0.485 mmol) and 1.25 M hydrochloric acid in methanol (1.00 mL, 1.25 mmol) in dichloromethane (6 mL) was stirred at ambient temperature for 30 minutes. The mixture was concentrated to dryness and the residue was precipitated from methanol with ether to yield the title compound as an off-white solid (0.191 g, 88%): ¹H NMR (300 MHz, DMSO-d₆) δ 11.18 (br s, 1H), 9.12 (q, J=4.0 Hz, 1H), 8.43 (d, J=5.8 Hz, 1H), 8.31 (s, 1H), 7.86-7.76 (m, 1H), 7.66 (d, J=5.8 Hz, 1H), 7.01 (d, J=10.1 Hz, 1H), 6.97-6.87 (m, 1H), 4.10 (d, J=13.4 Hz, 2H), 3.34 (t, J=11.1 Hz, 2H), 2.89 (s, 2H), 2.80 (d, J=4.0 Hz, 3H), 1.98 (d, J=13.4 Hz, 2H), 1.78 (t, J=11.1 Hz, 2H); ¹³C NMR (75 MHz, DMSO-d₆) δ 190.0, 166.9 (d, J_{C-F}=252.9 Hz), 163.4, 160.3 (d, J_{C-F}=14.0 Hz), 153.2, 151.4, 147.5, 140.8, 128.7 (d, J_{C-F}=11.6 Hz), 117.6 (d, J_{C-F}=2.2 Hz), 115.2, 114.0, 109.3 (d, J_{C-F}=22.8 Hz), 1051 (d, J_{C-F}=24.6 Hz), 79.0, 46.3, 33.2, 26.4; MS (ES+) m/z 413.0 (M+1).

### Example 1.1

### Synthesis of 7-fluoro-N-(4-(methylcarbamoyl)pyridin-2-yl)spiro[chroman-2,4′-piperidine]-1′-carboxamide

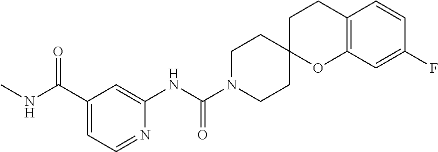

Following the procedure as described in Step A of EXAMPLE 1, making non-critical variations as required to replace 2-(7-fluoro-4-oxospiro[chroman-2,4′-piperidine]-t-ylcarboxamido)isonicotinic acid with 2-(7-fluorospiro[chroman-2,4′-piperidine]-1′-ylcarboxamido)isonicotinic acid, the title compound was obtained as a colorless solid in 72% yield: mp 177-179° C.; ¹H NMR (300 MHz, CDCl₃) δ 8.30 (d, J=51 Hz, 1H), 8.26 (s, 1H), 7.43 (dd, J=5.1, 1.5 Hz, 1H), 7.40 (s, 1H), 7.05-6.97 (m, 1H), 6.64-6.54 (m, 2H), 6.45 (br s, 1H), 3.93 (d, J=13.5 Hz, 2H), 3.47-3.34 (m, 2H), 2.99 (d, J=4.9 Hz, 3H), 1.76 (t, J=6.7 Hz, 2H), 1.96-1.79 (m, 4H), 1.70-1.57 (m, 2H); ¹³C NMR (75 MHz, CDCl₃) δ 166.3, 162.0 (d, J_{C-F}=243.4 Hz), 133 (d, J_{C-F}=11.8 Hz), 153.7, 153.3, 148.5, 143.9, 130.3 (d, J_{C-F}=9.6 Hz), 116.9, 116.6 (d, J_{C-F}=3.0 Hz), 109.5, 107.5 (d, J_{C-F}=21.6 Hz), 104.2 (d, J_{C-F}=24.0 Hz), 72.7, 40.1, 34.3, 31.9, 26.8, 20.7; MS (ES+) m/z 399.0 (M+1).

### Example 1.2

### Synthesis of 8-chloro-N-(4-(methylcarbamoyl)pyridin-2-yl)spiro[chroman-2,4′-piperidine]-1′-carboxamide hydrochloride

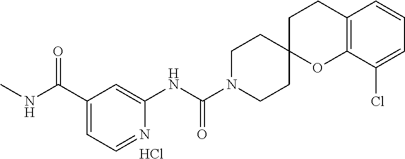

A. Following the procedure as described in Step A of EXAMPLE 1, making non-critical variations as required to replace 2-(7-fluoro-4-oxospiro[chroman-2,4′-piperidine]-1′-ylcarboxamido)isonicotinic acid with 2-(8-chlorospiro[chroman-2,4′-piperidine]-1′-ylcarboxamido)isonicotinic acid, 8-chloro-N-(4-(methylcarbamoyl)pyridin-2-yl)spiro[chroman-2,4′-piperidine]-1′-carboxamide was obtained as a colorless foam in 76% yield ¹H NMR (300 MHz, CDCl₃) δ 8.30 (d, J=5.1 Hz, 1H), 8.27 (s, 1H), 7.43 (dd, J=5.1, 1.3 Hz, 1H), 7.40 (br s, 1H), 7.20 J=7.8 Hz, 1H), 8.98 (d, J=7.8 Hz, 1H), 6.84-6.77 (m, 1H), 6.47 (br s, 1H), 3.98 (d, J=12.9 Hz, 2H), 3.48 J=12.9 Hz, 2H), 2.99 (d, J=4.9 Hz, 3H), 2.83 (t, J=6.8 Hz, 2H), 1.93 (d, J=12.9 Hz, 2H), 1.87 (t, J=6.8 Hz, 2H), 1.71-1.58 (m, 2H); MS (ES+) m/z 415.0 (M+1), 417.0 (M+1).

B. Following the procedure as described in Step B of EXAMPLE 1, making non-critical variations as required to replace 7-fluoro-N-(4-(methylcarbamoyl)pyridin-2-yl)-4-oxospiro[chroman-2,4′-piperidine]-1′-carboxamide with 8-chloro-N-(4-(methylcarbamoyl)pyridin-2-yl)spiro[chroman-2,4′-piperidine]-1′-carboxamide, the title compound was obtained as a colorless solid in 78% yield: ¹H NMR (300 MHz, DMSO-d₆) δ 11.10 (br s, 1H), 9.14-9.07 (m, 1H), 8.43 (d, J=6.1 Hz, 1H), 8.29 (s, 1H), 7.64 (dd, J=6.1, 1.4 Hz, 1H), 7.24 (dd, J=7.8, 1.2 Hz, 1H), 7.08 (dd, J=7.8, 1.2 Hz, 1H), 6.87-6.80 (m, 1H), 4.17 (d, J=13.3 Hz, 2H), 3.30 (t, J=11.4 Hz, 2H), 2.84-2.75 (m, 5H), 1.92-1.63 (m, 6H); ¹³C NMR (75 MHz, DMSO-d₆) δ 163.5, 153.3, 151.5, 148.1, 147.4, 140.8, 128.3, 127.5, 123.6, 120.9, 120.4, 115.2, 114.0, 74.0, 33.7, 30.5, 26.4, 20.8; MS (ES+) m/z 415.0 (M+1), 417.0 (M+1).

### Example 1.3

### Synthesis of 8-chloro-N-(4-(methylcarbamoyl)pyridin-2-yl)-4-oxospiro[chroman-2,4′-piperidine]-1′-carboxamide hydrochloride

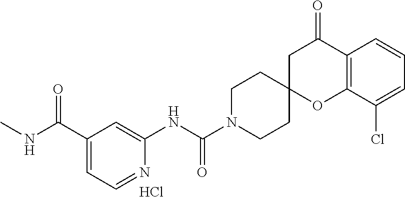

A. Following the procedure as described in Step A of EXAMPLE 1, making non-critical variations as required to replace 2-(7-fluoro-4-oxospiro[chroman-2,4′-piperidine]-1′-ylcarboxamido)isonicotinic acid with 2-(8-chloro-4-oxospiro[chroman-2,4′-piperidine]-1′-ylcarboxamido)isonicotinic acid, 8-chloro-N-(4-(methylcarbamoyl)pyridin-2-yl)-4-oxospiro[chroman-2,4′-piperidine]-1″-carboxamide was obtained as a yellowish foam in 68% yield: ¹H NMR (300 MHz, CDCl₃) δ 8.30 (d, J=5.1 Hz, 1H), 8.24 (s, 1H), 7.80 (dd, J=7.8, 1.6 Hz, 1H), 7.60 (dd, J=7.8, 1.6 Hz, 1H), 7.45 (br s, 1H), 7.43 (dd, J=5.1, 1.3 Hz, 1H), 7.05-7.95 (m, 1H), 6.46 (br s, 1H), 4.00 (d, J=13.5 Hz, 2H), 3.52-3.39 (m, 2H), 2.99 (d, J=4.9 Hz, 3H), 219 (s, 2H), 2.16 (d, J=13.5 Hz, 2H), 1.72 (td, J=13.5, 4.7 Hz, 2H); MS (ES+) m/z 429.0 (M+1), 431.0 (M+1).

B. Following the procedure as described in Step B of EXAMPLE 1, making non-critical variations as required to replace 7-fluoro-N-(4-(methylcarbamoyl)pyridin-2-yl)-4-oxospiro[chroman-2,4′-piperidine]-1′-carboxamide with 8-chloro-N-(4-(methylcarbamoyl)-pyridin-2-yl)-4-oxospiro[chroman-2,4′-piperidine]-1′-carboxamide, the title compound was obtained as a colorless solid in 88% yield: mp 215° C. (dec.); ¹H NMR (300 MHz, DMSO-d₆) δ 10.43 (br s, 1H), 8.94-8.85 (m, 1H), 8.39 (d, J=5.5 Hz, 1H), 8.17 (s, 1H), 7.79 (dd, J=7.8, 1.3 Hz, 1H), 7.72 (dd, J=7.8, 1.3 Hz, 1H), 7.50 (d, J=5.5 Hz, 1H), 7.14-7.05 (m, 1H), 4.11 (d, J=13.5 Hz, 2H), 3.25 (t, J=12.0 Hz, 2H), 2.96 (s, 2H), 2.80 (d, J=4.5 Hz, 3H), 2.00 (t, J=13.5 Hz, 2H), 1.85-1.71 (m, 2H); ¹³C NMR (75 MHz, DMSO-d₆) δ 190.7, 163.5, 153.5, 153.3, 151.5, 147.4, 141.0, 136.2, 124.8, 122.2, 121.8, 121.7, 115.2, 113.9, 79.4, 46.4, 33.1, 26.4; MS (ES+) m/z 429.0 (M+1), 431.0 (M+1).

### Example 1.4

### Synthesis of 6-fluoro-N-(4-(methylcarbamoyl)pyridin-2-yl)spiro[chroman-2,4′-piperidine]-1′-carboxamide

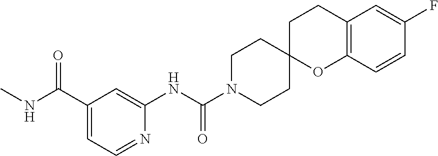

Following the procedure as described in Step A of EXAMPLE 1, making non-critical variations as required to replace 2-(7-fluoro-4-oxospiro[chroman-2,4′-piperidine]-1′-1′-ylcarboxamido)isonicotinic acid with 2-(6-fluorospiro[chroman-2,4′-piperidine]-1′-ylcarboxamido)isonicotinic acid, the title compound was obtained as a colorless solid in 62% yield; mp 175-176° C.; ¹H NMR (300 MHz, CDCl₃) δ 8.30 (d, J=4.9 Hz, 1H), 8.27 (s, 1H), 7.46-7.37 (m, 2H), 6.87-67.4 (m, 3H), 6.43 (br s, 1H), 3.92 (d, J=13.4 Hz, 2H), 3.40 (t, J=12.3 Hz, 2H), 2.99 (d, J=4.8 Hz, 3H), 2.79 (t, J=6.6 Hz, 2H), 1.90 (d, J=−13.4 Hz, 2H), 1.81 (t, J=6.6 Hz, 2H), 1.69-1.54 (m, 2H); ¹³C NMR (75 MHz, CDCl₃) δ 166.3, 156.7 (d, J_{C-F}=238.2 Hz), 153.8, 153.3, 148.7 (d, J_{C-F}=1.8 Hz), 148.4, 143.9, 122.1 (d, J_{C-F}=7.4 Hz), 118.0 (d, J_{C-F}=8.1 Hz), 116.8, 115.3 (d, J_{C-F}=22.4 Hz), 114.2 (d, J_{C-F}=23.1 Hz), 109.5, 72.3, 40.1, 34.1, 31.7, 26.7, 21.5; MS (ES+) m/z 399.0 (M+1).

### Example 1.5

### Synthesis of 7-fluoro-N-(6-(methylcarbamoyl)pyridin-2-yl)spiro[chroman-2,4′-piperidine]-it-carboxamide

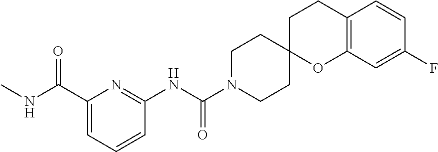

Following the procedure as described in EXAMPLE 1, making non-critical variations as required to replace 2-(7-fluoro-4-oxospiro[chroman-2,4′-piperidine]-1′-ylcarboxamido)isonicotinic acid with 6-(7-fluorospiro[chroman-2,4′-piperidine]-1′-ylcarboxamido)picolinic acid, the title compound was obtained as a colorless solid in 37% yield: mp 126-128° C.; ¹H NMR (300 MHz, CDCl₃) δ 8.16 (d, J=7.8 Hz, 1H), 7.87-7.76 (m, 2H), 7.72 (br s, 1H), 7.17 (br s, 1H), 6.64-6.54 (m, 2H), 3.94 (d, J=13.1 Hz, 2H), 3.41 (t, J=11.8 Hz, 2H), 3.00 (d, J=4.9 Hz, 3H), 2.76 (t, J=6.7 Hz, 2H), 1.91 (d, J=13.1 Hz, 2H), 1.83 (t, J=6.7 Hz, 2H), 1.71-1.59 (m, 2H); ¹³C NMR (75 MHz, CDCl₃) δ 164.6, 162.0 (d, J_{C-F}=243.4 Hz), 153.8 (d, J_{C-F}=11.8 Hz), 153.6, 151.3, 147.8, 139.4, 130.3 (d, J_{C-F}=9.6 Hz), 116.8, 116.7 (d, J_{C-F}=3.1 Hz), 115.9, 107.5 (d, J_{C-F}=21.5 Hz), 104.2 (d, J_{C-F}=24.0 Hz), 72.8, 40.1, 34.3, 31.9, 26.0, 20.7; MS (ES+) m/z 399.0 (M+1).

### Example 1.6

### Synthesis of 7-fluoro-N-(5-(methylcarbamoyl)pyridin-3-yl)spiro[chroman-2,4′-piperidine]-1′-carboxamide

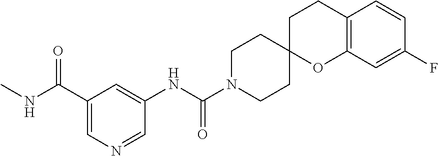

Following the procedure as described in EXAMPLE 1, making non-critical variations as required to replace 2-(7-fluoro-4-oxospiro[chroman-2,4′-piperidine]-1′-ylcarboxamido)isonicotinic acid with 5-(7-fluorospiro[chroman-2,4′-piperidine]-1′-ylcarboxamido)nicotinic acid, the title compound was obtained as a colorless solid in 68% yield: mp 231-233° C.; ¹H NMR (300 MHz, DMSO-d₆) δ 8.91 (s, 1H), 8.78 (br s, 1H), 8.61-8.53 (m, 2H), 8.29-8.26 (m, 1H), 7.16-7.09 (m, 1H), 6.71-6.62 (m, 2H), 3.91 (d, J=13.5 Hz, 2H), 3.31-3.22 (m, 2H), 27.9 (d, J=4.5 Hz, 3H), 2.72 (t, J=6.6 Hz, 2H), 1.82 (t, J=6.6 Hz, 2H), 1.74 (d, J=13.5 Hz, 2H), 1.69-1.56 (m, 2H); ¹³C NMR (75 MHz, DMSO-d₆) δ 165.2, 161.1 (d, J_{C-F}=240.5 Hz), 154.4, 153.8 (d, J_{C-F}=12.1 Hz), 143.0, 140.8, 137.2, 130.6 (d, J_{C-F}=9.7 Hz), 129.1, 125.2, 117.5 (d, J_{C-F}=2.9 Hz), 106.8 (d, J_{C-F}=21.2 Hz), 103.6 (d, J_{C-F}=23.8 Hz), 73.4, 39.6, 33.8, 30.5, 26.2, 20.1; MS (ES+) ink 399.0 (M+1).

### Example 1.7

### Synthesis of N-(4-(methylcarbamoyl)pyridin-2-yl)-1,3-dihydrospiro[indene-2,4′-piperidine]-1′-carboxamide

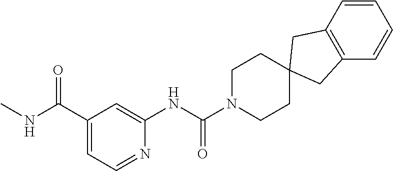

Following the procedure as described for 7-fluoro-N-(4-(methylcarbamoyl)pyridin-2-yl)-4-oxospiro[chroman-2,4′-piperidine]-1′-carboxamide, making non-critical variations as required to replace 2-(7-fluoro-4-oxospiro[chroman-2,4′-piperidine]-1-ylcarboxamido)isonicotinic acid with 2-(1,3-dihydrospiro[indene-2,4′-piperidine]-1′-ylcarboxamido)isonicotinic acid, the title compound was obtained as a colorless solid in 84% yield: mp 229-231° C.; ¹H NMR (300 MHz, CDCl₃) δ 8.30 (d, J=5.1 Hz, 1H), 8.27 (s, 1H), 7.43 (dd, J=5.1, 1.4 Hz, 1H), 7.40 (s, 1H), 7.23-7.12 (m, 4H), 6.53 (br s, 1H), 3.61-3.54 (m, 4H), 2.99 (d, J=4.8 Hz, 3H), 2.87 (s, 4H), 1.75-1.66 (m, 4H); ¹³C NMR (75 MHz, CDCl₃) δ 166.3, 153.8, 153.3, 148.4, 143.8, 141.6, 126.4, 124.8, 116.8, 109.6, 44.2, 42.2, 41.9, 36.5, 26.7, MS (ES+) m/z 365.1 (M+1).

The following Examples were prepared following the procedures as described in the above reaction schemes and examples or known by one skilled in the art.

| No. | Chemical Name | MW |
|---|---|---|
| 1.8 | N-(4-(methylcarbamoyl)pyridin-2-yl)spiro[chroman-2,4′- | 380.44 |
| | piperidine]-1′-carboxamide | |
| 1.9 | 7-fluoro-N-(4-(methylcarbamoyl)pyridin-2-yl)-3,4- | 398.43 |
| | dihydro-1H-spiro[naphthalene-2,4′-piperidine]-1′- | |
| | carboxamide | |
| 1.10 | 8-chloro-N-(4-(methylcarbamoyl)pyridin-2-yl)-3,4- | 412.91 |
| | dihydro-1H-spiro[naphthalene-2,4′-piperidine]-1′- | |
| | carboxamide | |
| 1.11 | 6-fluoro-N-(4-(methylcarbamoyl)pyridin-2-yl)-3,4- | 398.43 |
| | dihydro-1H-spiro[naphthalene-2,4′-piperidine]-1′- | |
| | carboxamide | |
| 1.12 | N-(4-(methylcarbamoyl)pyridin-2-yl)-3,4-dihydro-1H- | 378.47 |
| | spiro[naphthalene-2,4′-piperidine]-1′-carboxamide | |
| 1.13 | 6-fluoro-N-(4-(methylcarbamoyl)pyridin-2-yl)-3H- | 384.40 |
| | spiro[benzofuran-2,4′-piperidine]-1′-carboxamide | |
| 1.14 | 7-chloro-N-(4-(methylcarbamoyl)pyridin-2-yl)-3H- | 400.86 |
| | spiro[benzofuran-2,4′-piperidine]-1′-carboxamide | |
| 1.15 | 5-fluoro-N-(4-(methylcarbamoyl)pyridin-2-yl)-3H- | 384.40 |
| | spiro[benzofuran-2,4′-piperidine]-1′-carboxamide | |
| 1.16 | N-(4-(methylcarbamoyl)pyridin-2-yl)-3H- | 366.41 |
| | spiro[benzofuran-2,4′-piperidine]-1′-carboxamide | |
| 1.17 | 4-chloro-N-(4-(methylcarbamoyl)pyridin-2-yl)-1,3- | 382.43 |
| | dihydrospiro[indene-2,4′-piperidine]-1′-carboxamide | |
| 1.18 | 5-fluoro-N-(4-(methylcarbamoyl)pyridin-2-yl)-1,3- | 398.89 |
| | dihydrospiro[indene-2,4′-piperidine]-1′-carboxamide | |

### Example 2

### Synthesis of 6-fluoro-N-(4-{[(5-methyl-1,2-oxazol-3-yl)methyl]carbamoyl}pyridin-2-yl)-3,4-dihydro-1′H-spiro[chromene-2,4′-piperidine]-1′-carboxamide hydrochloride

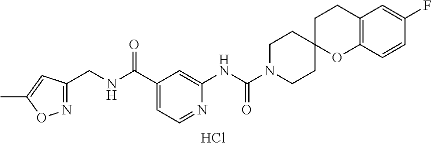

To a stirred mixture of 2-(6-fluorospiro[chroman-2,4′-piperidine]-1′-ylcarboxamido)isonicotinic acid (0.300 g, 0.778 mmol), 1-(5-methyl-isoxazol-3-yl)methaneamine hydrobromide (0.150 g, 0.777 mmol), 1-hydroxybenzotriazole (0.158 g 1.169 mmol) and 1-ethyl-3-(3-dimethylaminopropyl)carbodimide hydrochloride (0.224 g, 1.168 mmol) in N,N-dimethylformamide (3 mL) was added N,N-diisopropylethylamine (0.550 mL, 3.157 mmol). The reaction mixture was stirred at ambient temperature for 20 hours, diluted with ethyl acetate (75 mL), washed with saturated aqueous sodium bicarbonate solution (2×50 mL) and water (2×50 mL). The organic layer was dried over anhydrous sodium sulfate, filtered and concentrated in vacuo to afford an off-white foam. The foam was dissolved in dichloromethane (10 mL), stirred with 4 M hydrochloric acid in dioxane (0.5 mL) at ambient temperature for 30 minutes, and then concentrated. The residue was dissolved in methanol and precipitated with ether. The precipitate was collected by filtration and dried in a vacuum oven at 70° C. overnight to afford the title compound as a colorless solid (0.257 g, 84%): ¹H NMR (300 MHz, DMSO-d₆) δ 9.96 (br s, 1H) 9.42 (t, J=5.6 Hz, 1H), 8.38 (d, J=5.1 Hz, 1H), 8.17 (s, 1H), 7.45 (d, J=5.1 Hz, 1H), 6.98-6.88 (m, 2H), 6.84-6.78 (m, 1H), 6.17 (s, 1H), 4.46 (d, J=5.6 Hz, 2H), 3.96 (d, J=13.5 Hz, 2H), 3.28 (t, J=11.4 Hz, 2H), 2.75 (t, J=6.7 Hz, 2H), 2.37 (s, 3H), 1.79 (t, J=6.7 Hz, 2H), 1.73 (d, J=13.5 Hz, 2H), 1.68-1.55 (m, 2H); ¹³C NMR (75 MHz, DMSO-d₆) δ 169.5, 164.1, 161.6, 155.8 (d J_{C-F}=235.4 Hz), 153.6, 152.6, 148.9 (d, J_{C-F}=1.6 Hz), 145.3, 143.4, 122.9 (d, J_{C-F}=7.6 Hz), 117.8 (d, J_{C-F}=8.2 Hz) 115.2 (d, J_{C-F}=22.3 Hz), 115.1, 113.8 (d, J_{C-F}=23.0 Hz), 113.2, 101.2, 72.7, 39.9, 35.0, 33.7, 30.2, 20.9, 11.7; MS (ES+) m/z 480.1 (M+1).

### Example 2.1

### Synthesis of 6-fluoro-N-(4-{[(1-methyl-1H-pyrazol-4-yl)methyl]carbamoyl}pyridin-2-yl)-3,4-dihydro-1′H-spiro[chromene-2,4′-piperidine]-1′-carboxamide hydrochloride

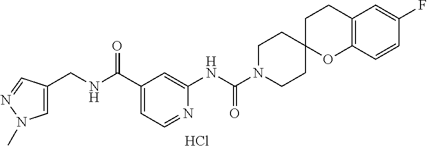

Following the procedure as described in EXAMPLE 2, making non-critical variations as required to replace 1-(5-methyl-isoxazol-3-yl)methaneamine hydrobromide with (1-methyl-1H-pyrazol-4-yl)methaneamine hydrochloride, the title compound was obtained as a colorless solid in 62% yield: ¹H NMR (300 MHz, DMSO-d₆) δ 10.28 (br s, 1H), 9.25 (t, J=5.6 Hz, 1H), 8.37 (d, J=5.7 Hz, 1H), 8.15 (s, 1H), 7.62 (s, 1H), 7.49 (d, J=5.7 Hz, 1H), 7.36 (s, 1H), 6.98-6.87 (m, 2H), 6.83-6.77 (m, 1H), 4.28 (d, J=5.6 Hz, 2H), 3.88 (d, J=13.4 Hz, 2H), 3.78 (s, 3H), 3.29 (t, J=11.1 Hz, 2H), 2.74 (t, J=6.5 Hz, 2H), 1.84-1.69 (m, 4H), 1.68-1.56 (m, 2H); ¹³C NMR (75 MHz, DMSO-d₆) δ 163.0, 155.8 (d, J_{C-F}=235.5 Hz), 153.3, 151.6, 148.8 (d, J_{C-F}=1.6 Hz), 147.2, 141.2, 137.9, 129.6, 122.9 (d, J_{C-F}=7.6 Hz), 118.1, 117.8 (d, J_{C-F}=8.2 Hz), 115.3, 115.2 (d, J_{C-F}=22.4 Hz), 114.0, 113.8 (d, J_{C-F}=22.8 Hz), 72.6, 38.3, 33.7, 30.2, 20.8; MS (ES+) m/z 479.1 (M+1).

### Example 3

### Synthesis of N-(4-carbamoylpyridin-2-yl)-6-fluorospiro[chroman-2,4′-piperidine]-1′-carboxamide

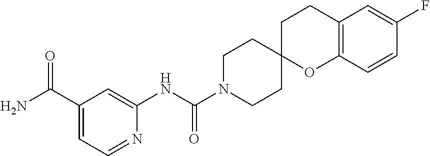

To a stirred mixture of 2-(6-fluorospiro[chroman-2,4′-piperidine]-1′-ylcarboxamido)isonicotinic acid (0.300 g, 0.778 mmol), ammonium chloride (0.231 g, 3.883 mmol), 1-hydroxybenzotriazole (0.158 g, 1.169 mmol) and 1-ethyl-3-(3-dimethylaminopropyl)carbodiimide hydrochloride (0.224 g, 1.168 mmol) in N,N-dimethylformamide (3 mL) was added N,N-diisopropylethylamine (1.10 mL, 6.315 mmol). The reaction mixture was stirred for 20 hours and then filtered. The collected solid was washed with saturated aqueous sodium bicarbonate solution, water and ethyl acetate, and then suspended in methanol and stirred at reflux for 30 minutes. The remaining solid was collected by filtration to afford the title compound as a colorless solid (0.190 g, 64%): mp 265-267° C.; ¹H NMR (300 MHz, DMSO-d₆) δ 9.38 (s, 1H), 8.32 (d, J=3.6 Hz, 1H), 8.13 (br s, 2H), 7.61 (s, 1H), 7.32 (d, J=3.6 Hz, 1H), 7.00-6.76 (m, 3H), 3.92 (d, J=12.6 Hz, 2H), 3.30-3.16 (m, 2H), 2.74 (br s, 2H), 1.88-1.51 (m, 6H); ¹³H NMR (75 MHz, DMSO-d₆) δ 166.8, 155.8 (d, J_{C-F}=235.2 Hz), 154.6, 154.3, 149.0 (d, J_{C-F}=1.6 Hz), 147.7, 143.1, 122.9 (d, J_{C-F}=7.6 Hz), 117.9 (d, J_{C-F}=8.2 Hz), 115.2 (d, J_{C-F}=22.3 Hz), 115.2, 113.8 (d, J_{C-F}=23.0 Hz), 111.8, 72.9, 39.7, 33.8, 30.3, 20.9; MS (ES+) m/z 385.0 (M+1).

### Example 4

### Synthesis of 7-fluoro-N-(4-methyl-5-(pyridin-2-ylmethylcarbamoyl)thiazol-2-yl)spiro[chroman-2,4′-piperidine]-1′-carboxamide

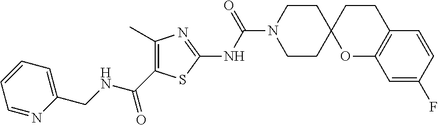

To a stirred suspension of 2-(7-fluorospiro[chroman-2,4′-piperidine]-1′-ylcarboxamido)-4-methylthiazole-5-carboxylic acid (0.240 g, 0.592 mmol) in dichloromethane (12 mL) at ambient temperature under nitrogen atmosphere was added N,N-dimethylformamide (2 drops), followed by the dropwise addition of oxalyl chloride (0.080 mL, 0.917 mmol). The resulting reaction mixture was stirred at ambient temperature for 1 hour and then concentrated to dryness. 2-(Aminomethyl)pyridine (0.100 mL, 0.978 mmol) and triethylamine (0.250 mL, 1.794 mmol) were combined in tetrahydrofuran (4 mL) at 0° C. To this cold solution was added dropwise the acid chloride in tetrahydrofuran (8 mL). After 10 minutes the cooling bath was removed, and reaction mixture was stirred at ambient temperature for 16 hours, quenched with saturated aqueous sodium bicarbonate solution (50 mL) and extracted with dichloromethane (2×50 mL). The combined organic layer was dried over anhydrous sodium sulphate, filtered and concentrated in vacuo. The residue was purified by preparative thin layer chromatography and eluted with 10% acetone in dichloromethane, and then triturated with ether/hexanes to afford the title compound as a colorless solid (0.130 g, 44%): mp 165-167° C.; ¹H NMR (300 MHz, CDCl₃) δ 9.36 (br s, 1H), 8.54 (d, 4.0 Hz, 1H), 7.71-7.61 (m, 1H), 7.32-7.14 (m, 3H), 7.04-6.94 (m, 1H), 6.62-6.50 (m, 2H), 4.70 (d, J=4.1 Hz, 2H), 3.93 (d, J=11.6 Hz, 2H), 3.37 (t, J=12.4 Hz, 2H), 2.74 (t, J=6.1 Hz, 2H), 2.60 (s, 3H), 1.87 (d, J=13.6 Hz, 2H), 1.80 (t, J=6.1 Hz, 2H), 1.66-1.53 (m, 2H); ¹³C NMR (75 MHz, CDCl₃) δ 162.4, 162.0 (d, J_{C-F}=243.4 Hz), 160.8, 155.7, 153.8, 153.7 (d, J_{C-F}=11.8 Hz), 150.0, 149.0, 136.7, 130.3 (d, J_{C-F}=9.6 Hz), 122.4, 121.9, 116.6 (d, J_{C-F}=3.0 Hz), 107.5 (d, J_{C-F}=21.6 Hz), 104.2 (d, J_{C-F}=24.1 Hz), 72.6, 44.6, 40.0, 34.1, 31.8, 20.7, 16.7; MS (ES+) m/z 496.0 (M+1).

### Example 5

### Measuring Stearoyl-CoA Desaturase Inhibition Activity of a Test Compound Using Mouse Liver Microsomes

The identification of compounds of the invention as SCD inhibitors was readily accomplished using the SCD microsomal assay procedure described in Shanklin J. and Summerville C., Proc. Natl. Acad. Sci. USA (1991), Vol. 88, pp. 2510-2514.

### Preparation of Mouse Liver Microsomes:

Male ICR outbread mice, on a high-carbohydrate, low fat diet, under light halothane (15% in mineral oil) anesthesia are sacrificed by exsanguination during periods of high enzyme activity. Livers are immediately rinsed with cold 0.9% NaCl solution, weighed and minced with scissors. All procedures are performed at 4′C unless specified otherwise. Livers are homogenized in a solution (1/3 w/v) containing 025 M sucrose, 62 mM potassium phosphate buffer (pH 7.0), 0.15 M KCL 15 mM N-acetyleysteine, 5 mM MgCl₂, and 0.1 mM EDTA using 4 strokes of a Potter-Elvehjem tissue homogenizer. The homogenate is centrifuged at 10,400×g for 20 min to eliminate mitochondria and cellular debris. The supernatant is filtered through a 3-layer cheesecloth and centrifuged at 105,000×g for 60 min. The microsomal pellet is gently resuspended in the same homogenization solution with a small glass/teflon homogenizer and stored at −70° C. The absence of mitochondrial contamination is enzymatically assessed. The protein concentration is measured using bovine serum albumin as the standard.

Incubation of Mouse Liver Microsomes with Test Compounds:

Desaturase activity is measured as the release of ³H₂O from [9,10-³H]stearoyl-CoA. Reactions per assay point conditions are as follows: 2 μL 1.5 mM stearoyl-CoA, 0.25 μL 1 mCi/mL ³H-stearoyl CoA, 10 μL 20 mM NADH, 36.75 μL 0.1 M PK buffer (K₂HPO₄/NaH₂PO₄, pH 7.2). The test compound or control solution is added in a 1 μL volume. Reactions are started by adding 50 μL of microsomes (1.25 mg/mL). The plates are mixed and after 15 min incubation on a heating block (25° C.), the reactions are stopped by the addition of 10 μL 60% PCA. An aliquot of 100 μL is then transferred to a fitter plate pretreated with charcoal and the plate centrifuged at 4000 rpm for 1 minute. The flow through containing the ³H₂O released by the SCD1 desaturation reaction is added to scintillation fluid and the radioactivity measured in a Packard TopCount. The data is analysed to identify the IC₅₀ for test compounds and reference compounds.

Representative compounds of the invention showed activity as inhibitors of SCD when tested in this assay. The activity was defined in terms of % SCD enzyme activity remaining at the desired concentration of the test compound or as the IC₅₀ concentration. The IC₅₀ (affinity) of the example compounds toward the stearoyl-CoA desaturase is comprised between around 20 mM and 0.0001 μM or between around 5 Wand 0.0001 μM or between around 1 μM and 0.0001 μM.

The following Table provides data that exemplifies representative compounds and their Microsomal IC₅₀ (μM) data.

### Example Activity Data

| | | Microsomal |
|---|---|---|
| Example | Compound name | IC₅₀ (μM) |
| 1 | 7-fluoro-N-(4-(methylcarbamoyl)pyridin-2- | 0.524 |
| | yl)-4-oxospiro[chroman-2,4′-piperidine]-1′- | |
| | carboxamide hydrochloride | |
| 1.1 | 7-fluoro-N-(4-(methylcarbamoyl)pyridin-2- | 0.017 |
| | yl)spiro[chroman-2,4′-piperidine]-1′- | |
| | carboxamide | |
| 1.2 | 8-chloro-N-(4-(methylcarbamoyl)pyridin-2- | 0.003 |
| | yl)spiro[chroman-2,4′-piperidine]-1′- | |
| | carboxamide hydrochloride | |
| 1.3 | 8-chloro-N-(4-(methylcarbamoyl)pyridin-2- | 0.056 |
| | yl)-4-oxospiro[chroman-2,4′-piperidine]-1′- | |
| | carboxamide hydrochloride | |
| 2.1 | 6-fluoro-N-(4-((1-methyl-1H-pyrazol-4- | 0.040 |
| | yl)methylcarbamoyl)pyridin-2- | |
| | yl)spiro[chroman-2,4′-piperidine]-1′- | |
| | carboxamide hydrochloride | |
| 4 | 7-fluoro-N-(4-methyl-5-(pyridin-2- | 0.048 |
| | ylmethylcarbamoyl)thiazol-2- | |
| | yl)spiro[chroman-2,4′-piperidine]-1′- | |
| | carboxamide | |

Those skilled in the art are aware of a variety of modifications to this assay that can be useful for measuring inhibition of stearoyl-CoA desaturase activity in microsomes or in cells by test compounds.

All of the U.S. patents. U.S. patent application publications, U.S. patent applications, foreign patents, foreign patent applications and non-patent publications referred to in this specification are incorporated herein by reference in their entireties.

From the foregoing it will be appreciated that although specific embodiments of the invention have been described herein for purposes of illustration, various modifications may be made without deviating from the spirit and scope of the invention. Accordingly, the invention is not limited except as by the appended claims.

1. A compound represented by Formula (I): 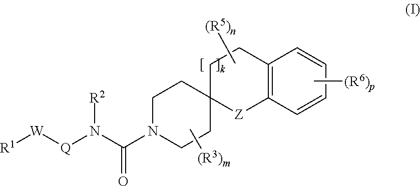
wherein Q is 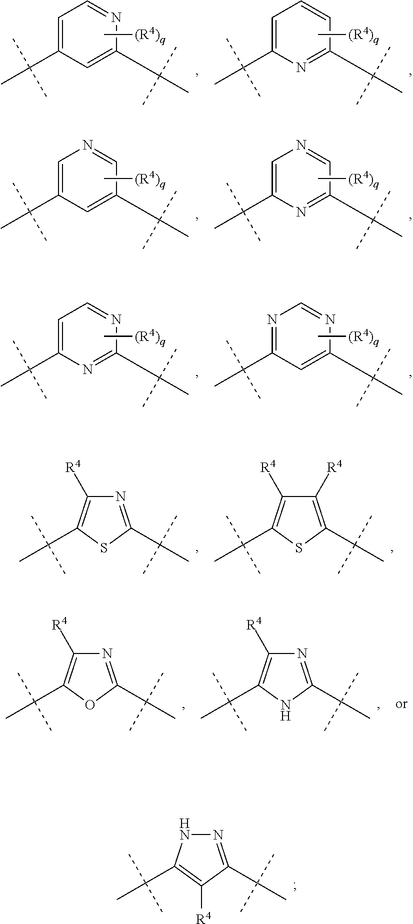
W is —N(R⁷)C(O)—, —C(O)N(R⁷)—, —N(R⁷)C(O)N(R⁷)—, —N(R⁷)S(O)ₜ—, —S(O)ₜN(R⁷)—, or a direct bond;
Z is —C(R⁴)ᵤ—, —C(O)—, —O—, N(R⁷)—, —S(O)ₜ, —O—, or —S—;
k is 0 or 1;
m is 0 to 8;
n is 0, 1, 2, 3 or 4;
p is 0, 1, 2, 3 or 4;
q is 1, 2, or 3;
t is 1 or 2;
u is 1 or 2;
R¹ is hydrogen, alkyl, alkenyl, alkynyl, alkoxy, hydroxyalkyl, alkoxyalkyl, cycloalkyl, cycloalkylalkyl, aryl, haloalkyl, aralkyl, heterocyclyl, heterocyclylalkyl, heteroaryl, or heteroarylalkyl;
or R¹ is a multi-ring structure having 2 to 4 rings wherein the rings are independently cycloalkyl, heterocyclyl, aryl or heteroaryl and where some or all of the rings may be fused to each other;
R² is hydrogen, or alkyl;
R³ is independently alkyl, halo, haloalkyl, hydroxy, or —N(R⁷)₂;
R⁴ is independently alkyl, alkenyl, alkynyl, alkoxy, hydroxyalkyl, alkoxyalkyl, cycloalkyl, heterocyclyl, aryl, aralkyl, heteroaryl, halo, haloalkyl, haloalkoxy, cyano, hydroxy, or —N(R)₂;
R⁵ is independently alkyl, halo, haloalkyl, hydroxy, cycloalkyl, or —N(R⁷)₂;
or two R⁵'s on the same carbon atom form an oxo (═O);
R⁶ is independently alkyl, alkenyl, alkynyl, alkoxy, hydroxyalkyl, alkoxyalkyl, cycloalkyl, cycloalkylalkyl, heterocyclyl, aryl, aralkyl, heteroaryl, halo, haloalkyl, haloalkoxy, cyano, hydroxy, or —N(R⁷)₂; and
R⁷ is independently hydrogen, alkyl, hydroxyalkyl, cycloalkyl, cycloalkylalkyl, aryl, heteroaryl, heterocyclyl or aralkyl; or
a pharmaceutically acceptable salt thereof.

2. The compound according to claim 2, or a pharmaceutically acceptable salt thereof, wherein
R¹ is hydrogen, C₁-C₄alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, C₆-C₁₀arC₁-C₄alkyl, C₁-C₁₀heterocyclyl, C₁-C₁₀heterocyclylC₁-C₄alkyl, C₁-C₁₀heteroaryl, or C₁-C₁₀heteroarylC₁-C₄alkyl;
or R¹ is a multi-ring structure having 2 to 4 rings wherein the rings are independently cycloalkyl, heterocyclyl, aryl or heteroaryl and where some or all of the rings may be fused to each other;
R² is hydrogen, or C₁-C₄alkyl;
R³ is independently C₁-C₄alkyl, halo, haloC₁-C₄alkyl, hydroxy, or —N(R⁷)₂;
R⁴ is C₁-C₄alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₁-C₁₀heterocyclyl, C₆-C₁₀aryl, C₆-C₁₀arC₁-C₄alkyl, C₁-C₁₀heteroaryl, halo, trifluoromethyl, trifluoromethoxy, cyano, hydroxy, or —N(R⁷)₂;
R⁵ is independently C₁-C₄alkyl, halo, haloC₁-C₄alkyl, hydroxy, C₃-C₇cycloalkyl, or —N(R⁷)₂;
or two R⁵'s on the same carbon atom form an oxo (═O);
R⁶ is independently C₁-C₄alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₁-C₁₀heterocyclyl, C₆-C₁₀aryl, C₆-C₁₀arC₁-C₄alkyl, C₁-C₁₀heteroaryl, halo, trifluoromethyl, trifluoromethoxy, cyano, hydroxy, or —N(R⁷)₂; and
R⁷ is independently hydrogen, C₁-C₄alkyl, hydroxyC₁-C₄alkyl, C₃-C₇cycloalkyl, C₃-C₇cycloalkylC₁-C₄alkyl, C₆-C₁₀aryl, C₁-C₁₀heteroaryl, C₁-C₁₀heterocyclyl or C₆-C₁₀arC₁-C₄alkyl.

3. The compound according to claim 1, or a pharmaceutically acceptable salt thereof, wherein Q is

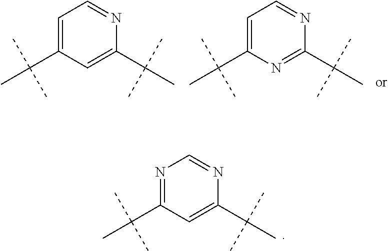

4. The compound according to claim 1, or a pharmaceutically acceptable salt thereof, wherein Q is

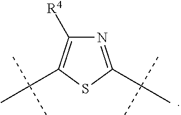

5. The compound according to claim 1, or a pharmaceutically acceptable salt thereof, wherein W is —N(R⁷)C(O)—, and
R¹ is hydrogen, or C₁-C₄alkyl.

6. The compound according to claim 1, or a pharmaceutically acceptable salt thereof, wherein W is —N(R⁷)C(O)—, and
R¹ is a C₁-C₆heteroarylC₁-C₄alkyl.

7. The compound according to claim 1, or a pharmaceutically acceptable salt thereof, wherein W is a direct bond or —N(R⁷)C(O)—, and R¹ is

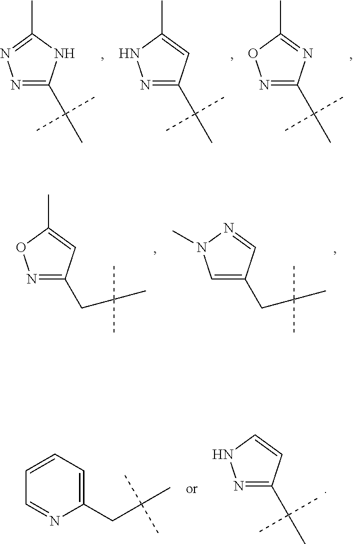

8. The compound according to claim 1, or a pharmaceutically acceptable salt thereof, wherein n is 1 and R⁵ is C₁-C₄alkyl, C₃-C₇cycloalkyl, or hydroxy; or n is 2 and two R⁵'s on the same carbon atom form an oxo (═O).

9. The compound according to claim 1, or a pharmaceutically acceptable salt thereof, wherein n is 2 and two R⁵'s on the same carbon atom form an oxo (═O).

10. The compound according to claim 1, or a pharmaceutically acceptable salt thereof, wherein p is 1 and R⁶ is C₁-C₄alkyl, C₃₋₇cycloalkyl, chloro, fluoro, trifluoromethyl, or cyano.

11. The compound according to claim 1, or a pharmaceutically acceptable salt thereof, wherein the compound is represented by Formula (II) 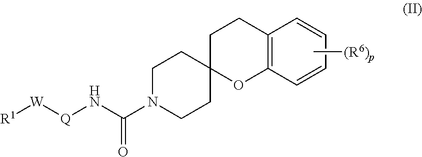
wherein Q is 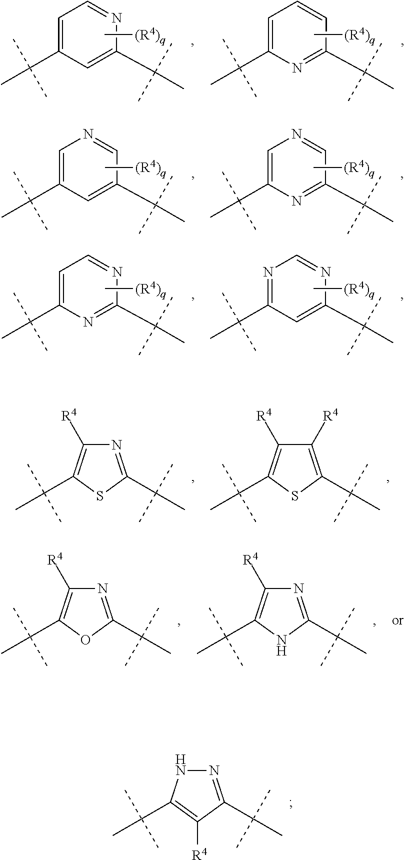
W is —N(R⁷)C(O)—, —C(O)N(R⁷)—, or a direct bond;
p is 0, 1, 2, 3, or 4;
q is 1, 2, or 3;
R¹ is hydrogen, alkyl, alkenyl, alkynyl, alkoxy, hydroxyalkyl, alkoxyalkyl, cycloalkyl, cycloalkylalkyl, aryl, haloalkyl, aralkyl, heterocyclyl, heterocyclylalkyl, heteroaryl, or heteroarylalkyl;
R⁴ is independently alkyl, alkoxy, hydroxyalkyl, alkoxyalkyl, cycloalkyl, halo, trifluoromethyl, trifluoromethoxy, cyano, hydroxy or —N(R⁷)₂;
R⁶ is independently C₁-C₄alkyl, halo, trifluoromethyl, trifluoromethoxy, cyano, hydroxy, or —N(R⁷)₂; and
R⁷ is independently hydrogen, or C₁-C₄alkyl; or
a pharmaceutically acceptable salt thereof.

12. The compound according to claim 8, or a pharmaceutically acceptable salt thereof, wherein
R¹ is hydrogen, C₁-C₄alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, C₆-C₁₀arC₁-C₄alkyl, C₁-C₁₀heterocyclyl, C₁-C₁₀heterocyclylC₁-C₄alkyl, C₁-C₁₀heteroaryl, or C₁-C₁₀heteroarylC₁-C₄alkyl; and
R⁴ is C₁-C₄alkyl, C₁-C₆alkoxy, hydroxyC₁-C₄alkyl, C₁-C₆alkoxyC₁-C₄alkyl, C₃-C₇cycloalkyl, halo, trifluoromethyl, trifluoromethoxy, cyano, hydroxy, or —N(R⁷)₂.

13. The compound according to claim 11, or a pharmaceutically acceptable salt thereof, wherein R¹ is C₁-C₄alkyl, cyclocalkylalkyl, aralkyl or heteroarylalkyl, wherein the cycloalkylalkyl is 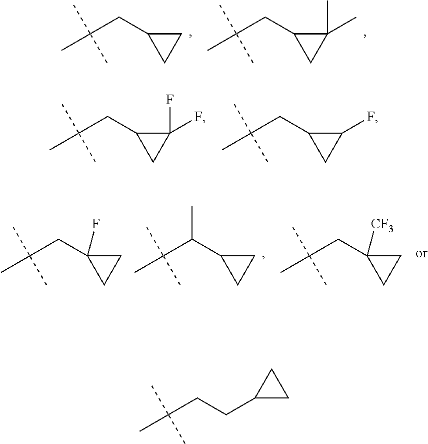
wherein the aralkyl is 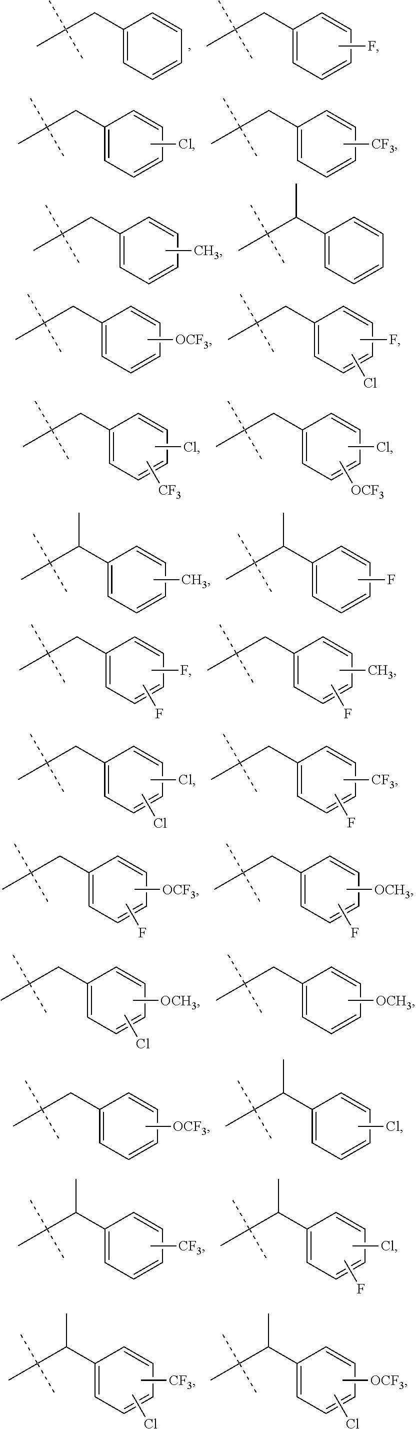 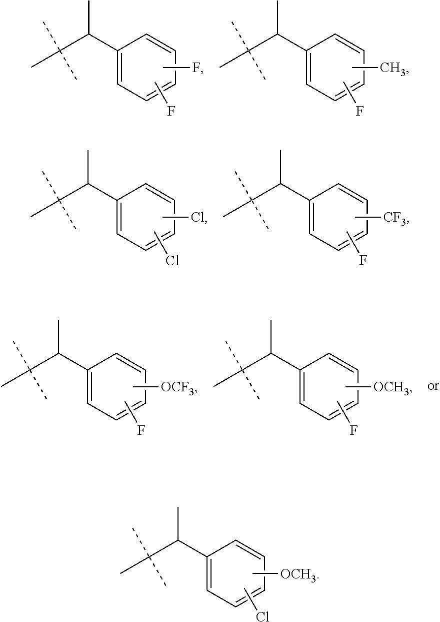
wherein the C₁-C₄alkyl is methyl,
ethyl, 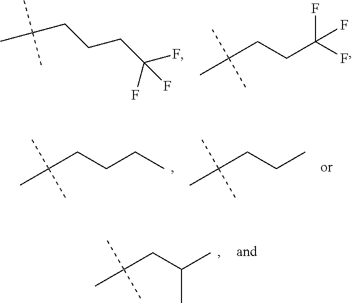
wherein the heteroarylalkyl is

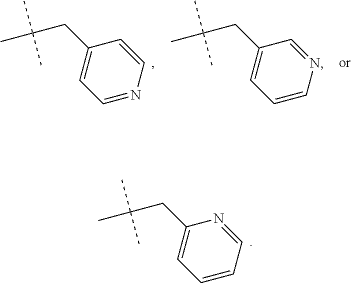

14. The compound according to claim 13, or a pharmaceutically acceptable salt thereof, wherein
R¹ is C₁-C₄alkyl, cyclocalkylalkyl, aralkyl or heteroarylalkyl,
wherein the cycloalkylalkyl is selected from the group consisting of 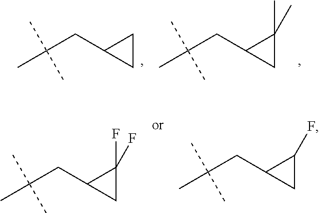
wherein the aralkyl is selected from the group consisting of 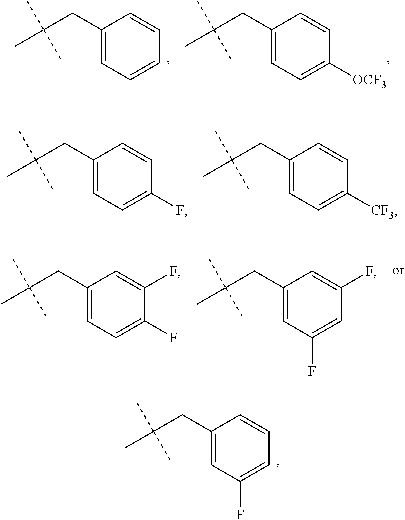
wherein the C₁-C₄ alkyl is selected from the group consisting of 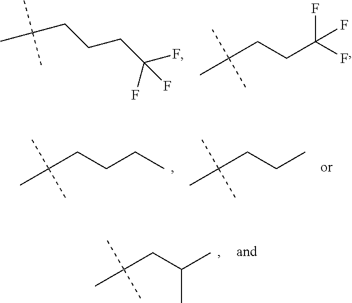
wherein the heteroarylalkyl is selected from the group consisting of

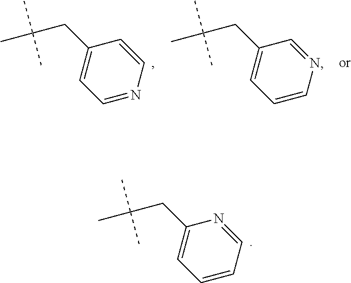

15. The compound according to claim 1, or a pharmaceutically acceptable salt thereof, wherein R¹ is hydrogen, methyl, (pyridin-2-yl)methyl, (5-methyl-isoxazol-3-yl)methyl, or (1-methyl-pyrazol-4-yl)methyl.

16. The compound according to claim 1, or a pharmaceutically acceptable salt thereof, wherein
W is —N(R⁷)C(O)—, and
R¹ is hydrogen,

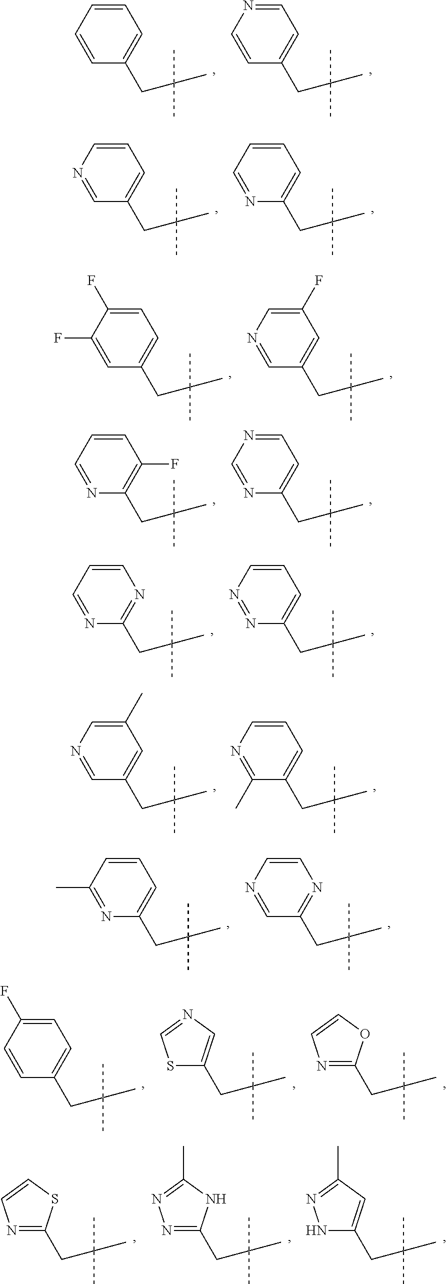

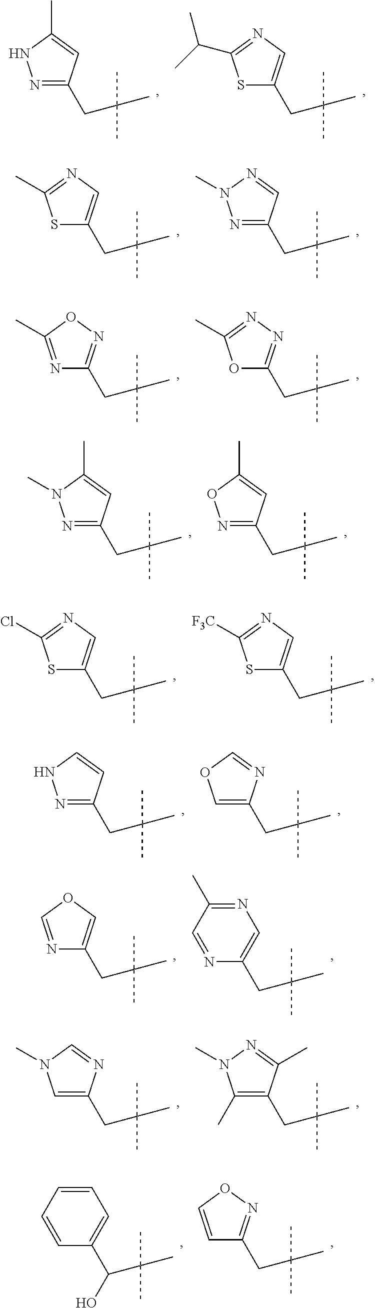

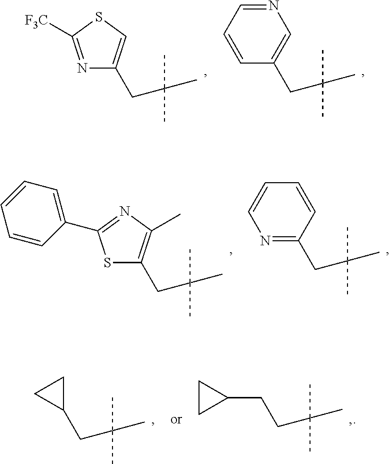

17. The compound according to claim 1, or a pharmaceutically acceptable salt thereof, wherein
W is a direct bond or —N(R⁷)C(O)—, and R¹ is

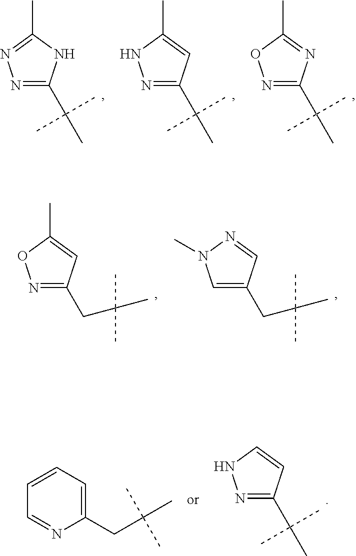

18. The compound according to claim 1, or a pharmaceutically acceptable salt thereof, wherein
W is —N(R⁷)C(O)—, and
R¹ is methyl.

19. The compound according to claim 1, or a pharmaceutically acceptable salt thereof, wherein k is 1.

20. The compound of claim 1, wherein the compound is selected from the group consisting of:
7-fluoro-N-(4-(methylcarbamoyl)pyridin-2-yl)-4-oxospiro[chroman-2,4′-piperidine]-1′-carboxamide;
7-fluoro-N-(4-(methylcarbamoyl)pyridin-2-yl)spiro[chroman-2,4′-piperidine]-1′-carboxamide;
8-chloro-N-(4-(methylcarbamoyl)pyridin-2-yl)spiro[chroman-2,4′-piperidine]-1′-carboxamide;
8-chloro-N-(4-(methylcarbamoyl)pyridin-2-yl)-4-oxospiro[chroman-2,4′-piperidine]-1′-carboxamide;
6-fluoro-N-(4-(methylcarbamoyl)pyridin-2-yl)spiro[chroman-2,4′-piperidine]-1′-carboxamide;
7-fluoro-N-(6-(methylcarbamoyl)pyridin-2-yl)spiro[chroman-2,4′-piperidine]-1′-carboxamide;
7-fluoro-N-(5-(methylcarbamoyl)pyridin-3-yl)spiro[chroman-2,4′-piperidine]-1′-carboxamide;
N-(4-(methylcarbamoyl)pyridin-2-yl)-1,3-dihydrospiro[indene-2,4′-piperidine]-1′-carboxamide;
N-(4-(methylcarbamoyl)pyridin-2-yl)spiro[chroman-2,4′-piperidine]-1′-carboxamide;
7-fluoro-N-(4-(methylcarbamoyl)pyridin-2-yl)-3,4-dihydro-1H-spiro[naphthalene-2,4′-piperidine]-1′-carboxamide;
8-chloro-N-(4-(methylcarbamoyl)pyridin-2-yl)-3,4-dihydro-1H-spiro[naphthalene-2,4′-piperidine]-1′-carboxamide;
6-fluoro-N-(4-(methylcarbamoyl)pyridin-2-yl)-3,4-dihydro-1H-spiro[naphthalene-2,4′-piperidine]-1′-carboxamide;
N-(4-(methylcarbamoyl)pyridin-2-yl)-3,4-dihydro-1H-spiro[naphthalene-2,4′-piperidine]-1′-carboxamide;
6-fluoro-N-(4-(methylcarbamoyl)pyridin-2-yl)-3H-spiro[benzofuran-2,4′-piperidine]-1′-carboxamide;
7-chloro-N-(4-(methylcarbamoyl)pyridin-2-yl)-3H-spiro[benzofuran-2,4′-piperidine]-1′-carboxamide;
5-fluoro-N-(4-(methylcarbamoyl)pyridin-2-yl)-3H-spiro[benzofuran-2,4′-piperidine]-1′-carboxamide;
N-(4-(methylcarbamoyl)pyridin-2-yl)-3H-spiro[benzofuran-2,4′-piperidine]-1′-carboxamide;
4-chloro-N-(4-(methylcarbamoyl)pyridin-2-yl)-1,3-dihydrospiro[indene-2,4′-piperidine]-1′-carboxamide;
5-fluoro-N-(4-(methylcarbamoyl)pyridin-2-yl)-1,3-dihydrospiro[indene-2,4′-piperidine]-1′-carboxamide;
6-fluoro-N-(4-{[(5-methyl-1,2-oxazol-3-yl)methyl]carbamoyl}pyridin-2-yl)-3,4-dihydro-1′H-spiro[chromene-2,4′-piperidine]-1′-carboxamide;
6-fluoro-N-(4-{[(1-methyl-1′H-pyrazol-4-yl)methyl]carbamoyl}pyridin-2-yl)-3,4-dihydro-1′H-spiro[chromene-2,4′-piperidine]-1′-carboxamide;
N-(4-carbamoylpyridin-2-yl)-6-fluorospiro[chroman-2,4′-piperidine]-1′-carboxamide;
7-fluoro-N-(4-methyl-5-(pyridin-2-ylmethylcarbamoyl)thiazol-2-yl)spiro[chroman-2,4′-piperidine]-1′-carboxamide; or
a pharmaceutically acceptable salt thereof.

21. A pharmaceutical composition, comprising: the compound according to claim 1, or a pharmaceutically acceptable salt thereof, and a pharmaceutically acceptable excipient or carrier.

22. A method of inhibiting human stearoyl-CoA desaturase (hSCD) activity comprising:
contacting a source of hSCD with a compound according to claim 1, or a pharmaceutically acceptable salt thereof.

23. A method of treating a disease or condition mediated by stearoyl-CoA desaturase (SCD) in a mammal, comprising:
administering to the mammal in need thereof a therapeutically effective amount of the compound according to claim 1, or a pharmaceutically acceptable salt thereof.

24. The method according to claim 23, wherein the disease or condition is metabolic syndrome, Syndrome X, diabetes, insulin resistance, decreased glucose tolerance, non-insulin-dependent diabetes mellitus, Type II diabetes, Type I diabetes, a diabetic complication, a body weight disorder, weight loss, body mass index or a leptin related disease.

25. The method according to claim 24, wherein the disease or condition is metabolic syndrome and the metabolic syndrome is dyslipidemia, obesity, insulin resistance, hypertension, microalbuminemia, hyperuricaemia or hypercoagulability.

26. The method according to claim 24, wherein the disease or condition is a body weight disorder selected from the group consisting of is obesity, overweight, cachexia and anorexia.

27. The method according to claim 23, wherein the disease or condition is a skin disorder.

28. The method according to claim 27, wherein the skin disorder is eczema, acne, psoriasis, rosacea, seborrheic skin or keloid scar formation or prevention.

29. A pharmaceutical composition comprising a therapeutically effective amount of a compound according to claim 1, or a pharmaceutically acceptable salt thereof, in combination with a therapeutically effective amount of insulin, an insulin derivative or mimetic;
an insulin secretagogue; an insulinotropic sulfonylurea receptor ligand; a PPAR ligand; an insulin sensitizer; a biguanide; an alpha-glucosidase inhibitor; GLP-1, a GLP-1 analog or mimetic; a DPPIV inhibitor; an HMG-CoA reductase inhibitor; a squalene synthase inhibitor; a FXR or LXR ligand; cholestyramine; a fibrate; nicotinic acid; or aspirin.

30-33. (canceled)
